(19) United States
(12) Patent Application Publication
(10) Pub. No.: US 2023/0051573 A1
(43) Pub. Date: Feb. 16, 2023

(54) METHODS AND KITS FOR DETECTING RHOA MUTATIONS

(71) Applicant: UNIVERSITY OF LEICESTER, Leicester Leicestershire (GB)

(72) Inventors: Simon WAGNER, Leicester Leicestershire (GB); Matthew AHEARNE, Leicester Leicestershire (GB); Barbara OTTOLINI, Leicester Leicestershire (GB)

(73) Assignee: UNIVERSITY OF LEICESTER, Leicester Leicestershire (GB)

(21) Appl. No.: 17/441,047

(22) PCT Filed: Mar. 19, 2020
(86) PCT No.: PCT/GB2020/050735

§ 371 Date: Sep. 20, 2021

(30) Foreign Application Priority Data
Mar. 20, 2019 (GB) 1903804.1

(51) Int. Cl. C12Q 1/6886

(52) U.S. Cl. CPC C12Q 1/6886; C12Q 2600/156; C12Q 2600/16; C12Q 1/6858

(57) ABSTRACT

The invention relates to methods of detecting mutations associated with the Ras homologue gene family member (RHOA) gene, and diagnosing conditions associated with these mutations, using Competitive allele-specific TaqMan polymerase chain reaction (cast-PCR). The invention also extends to the products used to detect mutations, and their use in diagnosis.

The present invention relates to methods of detecting mutations associated with the Ras homologue gene family member (RHOA) gene, and diagnosing conditions associated with these mutations. The invention also extends to the products used to detect mutations, and their use in diagnosis.

The accurate diagnosis of peripheral T-cell lymphoma (PTCL) is challenging, requiring morphologic interpretation, immunophenotyping, and molecular techniques. Establishing a precise diagnosis for this disease is critical for determining prognosis to and has the potential to impact both therapeutic decisions and clinical trial enrollment.

Lymphadenopathy can be a relatively minor clinical feature in the presentation of T-cell lymphoma and it is not uncommon for patients to have to undergo multiple biopsies in order to obtain a diagnosis, with all the associated risks and high psychological and physical distress for the patient. It is also recognized that interpretation of the histopathology and immunocytochemistry is difficult in these conditions, Taken together, all these difficulties are responsible for many discordant diagnoses, late referrals and inevitable impact on patient health. Genetic evidence that can be reliably and non-invasively obtained would enable valuable support for diagnosis and allow monitoring of response and prediction of relapse.

RHOA mutations are correlated to cancers, particularly PTCL. The inventors have developed an assay to identify patients with PTCL using allele-specific primers and oligoblockers or specific fluorescent probes enabling specific and sensitive detection of mutations in the RHOA gene sequence. In particular, from circulating cell-free nucleic acids to enable the detection of mutation associated with PTCL from any fluid sample containing cell free nucleic acids. The assay may also be performed on nucleic acids derived from circulating leucocytes, from any fluid.

Accordingly, in a first aspect of the invention, there is provided a polymerase chain reaction (PCR) method for determining the presence or absence of a mutation in the Ras homologue gene family member (RHOA) gene in a sample obtained from a subject, the method comprising:
i) forming a mixture comprising the sample and a primer set, wherein the sample comprises a RHOA encoding nucleotide target sequence and the primer set comprises;
   (a) a mutation-specific forward primer;
   (b) a reverse primer; and
   (c) an oligoblocker,
ii) subjecting the mixture of step (i) to, sequentially in steps, a denaturing step, an annealing step, and an extension step, wherein in the annealing step the forward primer and the oligoblocker compete to anneal to the target sequence, wherein the oligoblocker is capable of specifically hybridizing to a wild-type target sequence to block polymerase extension and the mutation-specific forward primer is capable of hybridizing to a mutated RHOA nucleotide sequence; and
iii) determining whether a PCR product is obtained through steps (i) and (ii), wherein the presence of the PCR product is indicative of the presence of a mutation in the RHOA gene in the sample, and the absence of the PCR product is indicative of the absence of a mutation in the RHOA gene in the sample.

The skilled person would understand that the mixture of step i) may comprise reagents that are well known in the art in performing the PCR reaction. For example, the mixture may further comprise a DNA polymerase enzyme, polynucleotides and a PCR reaction buffer.

The PCR reaction may be Quantitative (Q)-PCR, droplet-digital PCR and Crystal™ Digital™ PCR. All of these techniques are routine in molecular biology and known to those skilled in the art.

Such conditions used for PCR may be:
3 minutes at 95° C. to activate the DNA polymerase and initiate the denaturation process.
40/45 cycles of the following steps:
10 seconds at 95° C. for denaturation
30 seconds at 60° C. for the oligonucleotides hybridization
Signal read on the real-time instrument.
Melt curve from 60° C. to 90° C. with temperature gradient of 0.2° C. Read every 2 seconds.

Preferably, the PCR reaction is QPCR and detection of the PCR product is performed using an intercalating nucleic acid dye, such as SYBR green.

The sample may be a biological fluid sample, mononucleated white cells, polymorphonuclear leukocytes or tumour tissue.

Preferably the sample is a biological fluid that comprises cell free nucleic acids (cfNA). The skilled person would understand that cfNAs may be cell free DNA (cfDNA), cell free RNA, cell free siRNA and/or cell free miRNA. The invention may be conducted with any biological fluid sample containing cfNA, such as blood, plasma, serum, pleural fluid, saliva, urine, or the like. Preferably the sample is, or is derived from, blood, serum to or plasma.

The sample may be pretreated prior to being used in the invention (e.g., diluted, concentrated, separated, partially purified, frozen, etc.). Preferably the sample is a pretreated blood sample.

The wild-type RHOA amino acid sequence is provided herein, as follows:

[SEQ ID No: 125]
MAAIRKKLVIVGDGACGKTCLLIVFSKDQFPEVYVPTVFENYVADIEVD
GKQVELALWDTAGQEDYDRLRPLSYPDTDVILMCFSIDSPDSLENIPEK
WTPEVKHFCPNVPIILVGNKKDLRNDEHTRRELAKMKQEPVKPEEGRDM
ANRIGAFGYMECSAKTKDGVREVFEMATRAALQARRGKKKSGCLVL

The wild-type RHOA gene coding sequence is provided herein, as follows:

[SEQ ID No: 126]
gtggatgagctgtgagtgcgcgcgcgtgcgcggggccgcgacctgtgcc
ggctcgagcccgctgggcactcggaggcgcgcacgtcgttccccgccct
cccgccgccgcccgccctcgctctctcgcgctaccctcccgccgcccgc
ggtcctccgtcggttctctcgttagtccacggtctggtcttcagctacc
cgccttcgtctccgagtttgcgactcgcggaccggcgtccccggcgcga
agaggctggactcggattcgttgcctgagcaatggctgccatccggaag
aaactggtgattgttggtgatggagcctgtggaaagacatgcttgctca
tagtcttcagcaaggaccagttcccagaggtgtatgtgcccacagtgtt
tgagaactatgtggcagatatcgaggtggatggaaagcaggtagagttg
gctttgtgggacacagctgggcaggaagattatgatcgcctgaggcccc
tctcctacccagataccgatgttatactgatgtgtttttccatcgacag
ccctgatagtttagaaaacatcccagaaaagtggaccccagaagtcaag
catttctgtcccaacgtgcccatcatcctggttgggaataagaaggatc
ttcggaatgatgagcacacaaggcgggagctagccaagatgaagcagga
gccggtgaaacctgaagaaggcagagatatggcaaacaggattggcgct
tttgggtacatggagtgttcagcaaagaccaaagatggagtgagagagg
tttttgaaatggctacgagagctgctctgcaagctagacgtgggaagaa
aaaatctgggtgccttgtcttgtgaaaccttgctgcaagcacagccctt
atgcggttaattttgaagtgctgtttattaatcttagtgtatgattact
ggcctttttcatttatctataatttacctaagattacaaatcagaagtc
atcttgctaccagtatttagaagccaactatgattattaacgatgtcca
acccgtctggcccaccagggtccttttgacactgctctaacagccctcc
tctgcactcccacctgacacaccaggcgctaattcaaggaatttcttaa
cttcttgcttctttctagaaagagaaacagttggtaacttttgtgaatt
aggctgtaactactttataactaacatgtcctgcctattatctgtcagc
tgcaaggtactctggtgagtcaccacttcagggctttactccgtaacag
attttgttggcatagctctggggtgggcagttttttgaaaatgggctca
accagaaaagcccaagttcatgcagctgtggcagagttacagttctgtg
gtttcatgttagttaccttatagttactgtgtaattagtgccacttaat
gtatgttaccaaaaataaatatatctaccccagactagatgtagtattt
tttgtataattggatttcctaatactgtcatcctcaaagaaagtgtatt
ggttttttaaaaaagaaagtgtatttggaaataaagtcagatggaaaat
tcattttttaaattcccgttttgtcactttttctgataaaagatggcca
tattaccccttttcggccccatgtatctcagtaccccatggagctgggc
taagtaaataggaattggtttcacgcctgaggcaattagacactttgga
agatggcataacctgtctcacctggacttaagcatctggctctaattca
cagtgctcttttctcctcactgtatccaggttccctcccagaggagcca
ccagttctcatgggtggcactcagtctctcttctctccagctgactaaa
ctttttttctgtaccagttaatttttccaactactaatagaataaaggc
agttttctaaaaaaa

The mutation in the RHOA gene may be one or more nucleotide substitutions from the wild-type gene sequence, and preferably the mutation-specific primer contains the mutated position at one terminal nucleotide.

In one embodiment, the mutation is one or more nucleotide substitution in a coding (exon) region of the gene. Preferably the mutation is a single nucleotide substitution in a coding (exon) region of the gene.

Preferably, the mutation in the RHOA gene results in an amino acid substitution. Preferably, the substitution is selected from the group consisting of G14V, C16stop, G17V and F25L.

Thus, the mutation specific forward primer sequence may be capable of hybridizing to a mutated RHOA nucleic acid sequence comprising the substitution G14V, provided herein as follows:

[SEQ ID No: 127]
ccggaagaaactggtgattgttggtgatgtagcctgtggaaagacatgc
ttgctcatag

Thus, in a preferred embodiment, the primer sequences are capable of hybridizing to the nucleotide sequence as substantially as set out in SEQ ID No: 127, or a fragment or variant thereof.

The mutation specific forward primer sequence may be capable of hybridizing to the reverse complement sequence of the mutated RHOA nucleic acid sequence comprising the substitution G14V, provided herein, as follows:

[SEQ ID No: 128]
ctatgagcaagcatgtctttccacaggctacatcaccaacaatcaccag
tttcttccgg

Thus, in a preferred embodiment, the primer sequences are capable of hybridizing to the nucleotide sequence as substantially as set out in SEQ ID No: 128, or a fragment or variant thereof.

The mutation specific forward primer sequence may be capable of hybridizing to a mutated RHOA nucleic acid sequence comprising the substitution C16stop, provided herein, as follows:

[SEQ ID No: 129]
ccggaagaaactggtgattgttggtgatgaagcctgtggaaagacatgc
ttgctcatag

Thus, in a preferred embodiment, the primer sequences are capable of hybridizing to the nucleotide sequence as substantially as set out in SEQ ID No: 129, or a fragment or variant thereof.

The mutation specific forward primer sequence may be capable of hybridizing to the reverse complement sequence of the reverse complement sequence of the mutated RHOA nucleic acid sequence comprising the substitution C16stop, provided herein as follows:

[SEQ ID No: 130]
Ctatgagcaagcatgtctttccacaggcttcatcaccaacaatcaccag
tttcttccgg

Thus, in a preferred embodiment, the primer sequences are capable of hybridizing to the nucleotide sequence as substantially as set out in SEQ ID No: 130, or a fragment or variant thereof.

The mutation specific forward primer sequence may be capable of hybridizing to a mutated RHOA nucleic acid sequence comprising the substitution G17V, provided herein as follows:

[SEQ ID No: 131]
actggtgattgttggtgatggagcctgtgtaaagacatgcttgctcata
gtcttcagca

Thus, in a preferred embodiment, the primer sequences are capable of hybridizing to the nucleotide sequence as substantially as set out in SEQ ID No: 131, or a fragment or variant thereof.

The mutation specific forward primer sequence may be capable of hybridizing to the reverse complement of the mutated RHOA nucleic acid sequence comprising the substitution G17V, provided herein as follows:

[SEQ ID No: 132]
tgctgaagactatgagcaagcatgtctttacacaggctccatcaccaa
caatcaccagt

Thus, in a preferred embodiment, the primer sequences are capable of hybridizing to the nucleotide sequence as substantially as set out in SEQ ID No: 132, or a fragment or variant thereof.

The mutation specific forward primer sequence may be capable of hybridizing to a mutated RHOA nucleic acid sequence comprising the substitution F25L, provided herein as follows:

[SEQ ID No: 133]
cctgtggaaagacatgcttgctcatagtcctcagcaaggaccagttcc
cagaggtgtat

Thus, in a preferred embodiment, the primer sequences are capable of hybridizing to the nucleotide sequence as substantially as set out in SEQ ID No: 133, or a fragment or variant thereof.

The mutation specific forward primer sequence may be capable of hybridizing to the reverse complement of the mutated RHOA nucleic acid sequence comprising the substitution F25L, provided herein as follows:

[SEQ ID No: 134]
atacacctctgggaactggtccttgctgaggactatgagcaagcatgtc
tttccacagg

Thus, in a preferred embodiment, the primer sequences are capable of hybridizing to the nucleotide sequence as substantially as set out in SEQ ID No: 134, or a fragment or variant thereof.

The mutation-specific primer sequence may be a nucleotide sequence of any one of SEQ ID NO: 1-14 or a variant or fragment thereof, preferably any one of SEQ ID NO: 5-7, 11-16 or a variant or fragment thereof, and is capable of hybridizing to a mutated RHOA nucleotide sequence resulting in the amino acid substitution F25L.

The mutation-specific primer sequence may be a nucleotide sequence of any one of SEQ ID NO: 39-52 or a variant or fragment thereof, preferably any one of SEQ ID NO: 43-44, 50-51 or a variant or fragment thereof, and is capable of hybridizing to a mutated RHOA nucleotide sequence comprising a mutation resulting in the amino acid substitution G17V.

The mutation-specific primer sequence may be a nucleotide sequence of any one of SEQ ID NO: 72-85 or a variant or fragment thereof, preferably any one of SEQ ID NO: 75-78, 83-85 or a variant or fragment thereof, and is capable of hybridizing to a mutated RHOA nucleotide sequence resulting in the amino acid substitution C16stop.

The mutation-specific primer sequence may be a nucleotide sequence of any one of SEQ ID NO: 89-102 or a variant or fragment thereof, preferably any one of SEQ ID NO: 92-95, 99-101 or a variant or fragment thereof, and is capable of hybridizing to a mutated RHOA nucleotide sequence resulting in the amino acid substitution G14V.

The RHOA nucleotide sequence to which the primer sequences are capable of hybridizing to may be a DNA or RNA sequence, preferably DNA or RNA sequences derived from either cell-free nucleic acids or from peripheral blood mononuclear cells. Preferably the DNA or RNA sequences are derived from cell-free nucleic acids. The RNA sequence may be a miRNA, mRNA or siRNA sequence

The term “primer” designates, within the context of the present invention, a nucleotide sequence of that can hybridize specifically to a target genetic sequence and serve to initiate amplification. Primers of the invention may be a single-stranded nucleotide sequence , with a length of between 10 and 50 nucleotides between 10 and 40 nucleotides, between 10 and 30 nucleotides, between 10 and 25 nucleotides between 11 and 50, between 11 and 40 nucleotides, between 11 and 30 nucleotides, between 11 and 25 nucleotides, between 13 and 50, between 13 and 40 nucleotides, between 13 and 30 nucleotides, between 13 and 25 nucleotides, between 14 and 50, between 14 and 40 nucleotides, between 14 and 30 nucleotides, between 14 and 25 nucleotides, between 15 and 50, between 15 and 40 nucleotides, between 15 and 30 nucleotides, between 15 and 25 nucleotides. Preferably primers of the invention have a length of between 15 and 30 nucleotides. More preferably primers of the invention have a length of between 15 and 25.

Preferably, primers are perfectly matched with the targeted sequence in the RHOA nucleotide sequence, i.e. having 100% complementarity, allowing specific hybridization thereto and substantially no hybridization to another region.

In one embodiment, when the mutation is a nucleotide substitution, the mutation-specific primer contains the mutated position at one terminal nucleotide thereof. This confirmation advantageously, enables increased specificity of the method. In a preferred embodiment, the mutated position is at the 3′-terminal nucleotide of the mutation-specific primer, or at the 5′-terminal nucleotide of the mutation-specific primer.

The reverse primer is capable of hybridizing to a sequence in the RHOA gene which is non-mutated, i.e. wild-type, and amplifies, with its paired mutation-specific forward primer, the target sequence.

The skilled person would understand that the reverse primer can be chosen from any consecutive nucleic acid sequence, preferably DNA sequence, that is selected from the following regions: chr3: 49412715 to 49412925 or chr3: 49412975 tp 4913185 (human assembly GRCh37/hg19), according to the orientation of the mutation-specific forward primer.

Preferably, the target sequence that is amplified is about 10 to 200, 20 to 200, 30 to 200, 40 to 200, 50 to 200, 60 to 200, 70 to 200, 80 to 200, 90 to 200, 100 to 200, 150 to 200, 10 to 150, 10 to 100, 10 to 95, 10 to 90, 20 to 150, 20 to 100, 20 to 95, 20 to 90, 30 to 150, 30 to 100, 30 to 95, 30 to 90, 40 to 150, 40 to 100, 40 to 95, 40 to 90, 50 to 150, 50 to 100, 50 to 95, 50 to 90, 60 to 150, 60 to 100, 60 to 95, 60 to 90, 70 to 150, 70 to 100, 70 to 95, 70 to 90, 80 to 150, 80 to 100, 80 to 95 or 80 to 90 nucleotides in length. Preferably, the target sequence that is amplified is about 50 to 200 nucleotides in length. Most preferably, the target sequence that is amplified is about 50 to 95 nucleotides in length.

Primers of the invention may be a sequence with a GC content of between 40 and 60%. Such a level of GC content provides the optimized combination of specific amplification and efficacy. Preferably, the Tm of the primers is between 40 and 60° C., and preferably all of the primers have substantially the same Tm. The Tm difference between the mutation-specific forward primer and its reverse primer is preferably less than 5° C., more preferably less than 4° C., 3° C., 2° C. or 1° C.

Self-annealing may decrease the quantity of primer available for the amplification and reduce efficacy. Moreover, it contributes to non-specific amplification. Thus, in a to preferred embodiment, the primers of the invention are not self-annealing. The risk of self-annealing and self-hairpin loops of primers may be determined in silico using available software such as the software Oligoanalyzer®, which analyses the change in Gibbs free energy required for the breaking of secondary structures (ΔG). Typically, if this energy is negative, it should not be below −4 kcal/mol, otherwise risk of self-annealing or self-hairpin loops could occur. In a particular embodiment, primers of the invention have a ΔG above or equal to −4 kcal/mol as determined with Oligoanalyzer®. When using online tools as IDT OligoAnalyzer, which rely on different algorithms than Oligoanalyzer® for analyzing the change in Gibbs free energy required for the breaking of secondary structures, the ΔG is preferably above —7 kcal/mol otherwise risk of self-annealing or self-hairpin loops could occur. Accordingly, in another embodiment, primers of the invention have a ΔG above or equal to −7 kcal/mol as determined with IDT OligoAnalyzer.

The method of the first aspect comprises the use of an oligoblocker, which advantageously increases specificity of the method.

The skilled person would understand that an oligoblocker is an oligonucleotide which hybridizes specifically to the target sequence in non-mutated form, such hybridization results in a blockade of amplification. However, when the targeted mutation is present, the oligoblocker does not hybridize to the target sequence and amplification of the mutated sequence occurs. In contrast, when the mutation is not present, the oligoblocker hybridizes to the target sequence and prevents non-specific amplification.

The oligoblocker is capable of hybridizing specifically to the target sequence in non-mutated form. The skilled person would understand that “specifically” means that its sequence is exactly complementary to the non-mutated sequence. As a result, the oligoblocker does not hybridize to the gene when the mutation is present.

Preferably, the oligoblocker has a nucleotide length substantially the same as that of the mutation-specific forward primer and reverse primer sequences. It may therefore be between 10 and 50 nucleotides between 10 and 40 nucleotides, between 10 and 30 nucleotides, between 10 and 25 nucleotides between 11 and 50, between 11 and 40 nucleotides, between 11 and 30 nucleotides, between 11 and 25 nucleotides, between 13 and 50, between 13 and 40 nucleotides, between 13 and 30 nucleotides, between 13 and 25 nucleotides, between 14 and 50, between 14 and 40 nucleotides, between 14 and 30 nucleotides, between 14 and 25 nucleotides, between 15 and 50, between 15 and 40 nucleotides, between 15 and 30 nucleotides, between 15 and 25 nucleotides. Preferably the oligoblocker has a length of between 15 and 30 nucleotides. More preferably, the oligoblocker of the invention has a length of between 15 and 25

In order to increase specificity, the nucleotide of the mutation-specific primer that corresponds to the mutated nucleotide in the RHOA nucleotide sequence may be present in substantially the center nucleotide of the mutation-specific primer sequence. The nucleotide of the mutation-specific primer that corresponds to the mutated nucleotide in the RHOA nucleotide sequence may be located at a nucleotide that is within 20, 19, 18, 17, 16, 15, 14, 13, 12, 11, 10, 9, 8, 7, 6, 5, 4, 3, 2, or 1 nucleotides of the central nucleotide or nucleotides of the mutation-specific primer. Preferably, the nucleotide of the mutation-specific primer that corresponds to the mutated nucleotide in the RHOA nucleotide sequence is located at a nucleotide that is within 10 nucleotides of the central nucleotide or nucleotides of the mutation-specific primer. More preferably, the nucleotide of the mutation-specific primer that corresponds to the mutated nucleotide in the RHOA nucleotide sequence is located at a nucleotide that is within 5 nucleotides of the central nucleotide or nucleotides of the mutation-specific primer. Most preferably, the nucleotide of the mutation-specific primer that corresponds to the mutated nucleotide in the RHOA nucleotide sequence is located at a nucleotide that is within 1 nucleotides of the central nucleotide or nucleotides of the mutation-specific primer.

The oligoblocker may be modified, preferably terminally, to prevent amplification upon hybridization, e.g. to prevent polymerase elongation. As a result, when the mutation is not present, the oligoblocker hybridizes to the target sequence and prevents any non-specific amplification. In a preferred embodiment, the oligoblocker is phosphorylated 3′, resulting in a blockade of polymerase elongation.

The oligoblocker Tm may be about 60° C. (the hybridization/extension temperature of the polymerase enzyme required for the PCR amplification reaction) and preferably at least 1° C., 2° C., 3° C. or 4° C. above the Tm of the mutation-specific primer. This advantageously ensures that the oligoblocker hybridizes to its target sequence before any potential non-specific hybridization to the WT locus by the mutation-specific primer. Preferably, the oligoblocker Tm is between 55° C. and 60° C.

Where the mutated RHOA nucleotide sequence results in the amino acid substitution F25L, the oligoblocker may be selected in the following regions: chr3: 49412925-49412975 (human assembly GRCh37/hg19), where the position chr3: 49412950 is included in the selected oligoblocker sequence.

Where the mutated RHOA nucleotide sequence results in the amino acid substitution G17V, the oligoblocker may be selected in the following regions: chr3: 49412948-49412998 (human assembly GRCh37/hg19) where the position chr3: 49412973 is included in the selected oligoblocker sequence.

Where the mutated RHOA nucleotide sequence results in the amino acid substitution C16Stop, the oligoblocker may be selected in the following regions: chr3: 49412950-49413000 (human assembly GRCh37/hg19), where the position chr3: 49412975 is included in the selected oligoblocker sequence.

Where the mutated RHOA nucleotide sequence results in the amino acid substitution G14V, the oligoblocker may be selected in the following regions: chr3: 49412957-49413007 (human assembly GRCh37/hg19), where the position chr3: 49412982 is included in the selected oligoblocker sequence.

The method may be used to detect one or more mutations in the RHOA gene from a subject sample, either sequentially, or in parallel.

Accordingly, in one embodiment, mutated RHOA nucleotide sequence results in the amino acid substitution F25L, and the mutation-specific forward primer is selected from any one of SEQ ID NO:1-14, the reverse primer is selected from any one of SEQ ID NO: 15-20, and the oligoblocker is selected from any one of SEQ ID NO: 21-31, 34-38. Preferably the mutation-specific forward primer is selected from any one of SEQ ID NO: 5-7, 11-16, the reverse primer is selected from any one of SEQ ID NO: 15, 17-20, and the oligoblocker is selected from any one of SEQ ID NO: 23-24, 36-37; and/or the mutated RHOA nucleotide sequence results in the amino acid substitution G17V and the mutation-specific forward primer is selected from anyone of any one of SEQ ID NOs: 39-52, the reverse primer is selected from any one of SEQ ID NO: 53-59, and the oligoblocker is selected from any one of SEQ ID NO: 60-69. Preferably, the mutation-specific forward primer is selected from any one of SEQ ID NO: 43-44, 50-51, the reverse primer is selected from any one of SEQ ID NO: 53-54, 58-59, and the oligoblocker is selected from any one of SEQ ID NO: 62-64, 67-69; and/or

the mutated RHOA nucleotide sequence results in the amino acid substitution RHOA C16Stop and wherein the mutation-specific primer is selected from any one of SEQ ID NO: 72-85, the reverse primer is selected from any one of SEQ ID NO: 53-59, 86-88, and the oligoblocker is selected from any one of SEQ ID NO: 60-69. Preferably, the mutation-specific primer is selected from any one of SEQ ID NO: 75-78, 83-85, the reverse primer is selected from any one of SEQ ID NO: 53-54, 58-59, 86-88, and the oligoblocker is selected from any one of SEQ ID NO: 62-64, 67-69; and/or

the mutated RHOA nucleotide sequence results in the amino acid substitution RHOA G14V and the mutation-specific primer is selected from any one of SEQ ID NOs: 89-102, the paired primer is selected from any one of SEQ ID NO: 53-59, 86-88, and the oligoblocker is selected from SEQ ID NO: 60-69,103-112. Preferably the mutation-specific primer is selected from any one of SEQ ID NO: 92-95, 99-101, the paired primer is selected from any one of SEQ ID NO: 54, 58-59, 86-88, and the oligoblocker is selected from any one of SEQ ID NO: 62-64, 67-69,106-107, 111-112.

As an alternative to the use of a mutation-specific primer, mutation detection may be performed by specific hybridization of a fluorescent probe, configured to be complementary with the mutant allele of the RHOA gene.

Thus, in a second aspect there is provided a there is provided a polymerase chain reaction (PCR) method for determining the presence or absence of a mutation in the RHOA gene in a sample obtained from a subject, the method comprising:

i) forming a mixture comprising: the sample, a wild-type forward primer, a wild-type reverse primer, and a mutation specific fluorescent probe, wherein the sample comprises a RHOA encoding nucleotide target sequence;

ii) subjecting the mixture of step (i) to, sequentially in steps, a denaturing step, an annealing step, and an extension step, wherein the mutant specific fluorescent probe is capable of hybridizing to a mutated RHOA nucleotide sequence and the wild-type forward primer and wild-type reverse primer are capable of hybridizing to a wild-type RHOA encoding nucleotide target sequence; and

iii) detecting a fluorescent signal obtained by the reaction of step ii), wherein the presence of a fluorescent signal in a channel associated with the mutant specific fluorescent probe is indicative of the presence of a mutation in the RHOA gene, and the absence of a fluorescent signal is indicative of the absence of a mutation in the RHOA gene.

The mutation, sample, PCR reaction and RHOA nucleotide sequence may be as defined in the first aspect.

PCR reaction conditions may be as follows:
10 minutes at 95° C. for enzyme activation (ramp rate 2° C. per second)
40 cycles of the following steps (ramp rate 2° C. per second):
30 seconds at 94° C. for denaturation
1 minute at 55° C./60° C. for annealing/extension
10 minutes of enzyme deactivation at 98° C. (ramp rate 1° C. per second)

Preferably, the PCR reaction is performed on a droplet-digital PCR system.

The mutant specific fluorescent probe may be capable of hybridizing to a mutated RHOA nucleotide sequence as defined in the first aspect.

Thus, where the mutated RHOA nucleotide sequence results in the amino acid substitution RHOA F25L, the mutation specific fluorescent probe preferably comprises a nucleic acid sequence substantially as set out in SEQ ID NO: 118, or a fragment or variant thereof;

where the mutated RHOA nucleotide sequence results in the amino acid substitution G17V, the mutation specific fluorescent probe preferably comprises a nucleic acid sequence substantially as set out in SEQ ID NO: 120, or a fragment or variant thereof;

where the mutated RHOA nucleotide sequence results in the amino acid substitution C16Stop, the mutation specific fluorescent probe preferably comprises a nucleic acid sequence substantially as set out in SEQ ID NO: 122, or a fragment or variant thereof; and/or

where the mutated RHOA nucleotide sequence results in the amino acid substitution G14V, the mutation specific fluorescent probe preferably comprises a nucleic acid sequence substantially as set out in SEQ ID NO: 124, or a fragment or variant thereof.

The mutation specific fluorescent probe is preferably a single-strand nucleic acid sequence labelled 5′ or 3′ with a fluorophore group, such as 6-carboxyfluorescin (6FAM), HEX or VIC. Preferably the sequence is labelled 5′.

Preferably, the mutation specific fluorescent probe is a dual-labelled probe, such as a single-strand nucleic acid sequence labelled, preferably 5′, with a fluorophore group, such as 6-carboxyfluorescin (6FAM), HEX or VIC, and preferably labeled with a fluorescence quencher at the 3′ end. Preferably the quencher is Black Hole-1 (BHQ-1).

Preferably, the mutation specific fluorescent probe is between 10 and 30 nucleotides in length, between 10 and 25 nucleotides in length, between 10 and 20 nucleotides in length, between 11 and 30 nucleotides in length, between 11 and 25 nucleotides in length, between 11 and 20 nucleotides in length between 12 and 30 nucleotides in length, between 12 and 25 nucleotides in length, between 13 and 30 nucleotides in length, between 13 and 25 nucleotides in length, between 13 and 20 nucleotides in length between 14 and 30 nucleotides in length, between 14 and 25 nucleotides in length, between 15 and 30 nucleotides in length, between 15 and 25 nucleotides in length, between 15 and 20 nucleotides in length, between 20 and 30 nucleotides in length or between 25 and 30 nucleotides in length.

Preferably, the mutation specific fluorescent probe is between 10 and 30 nucleotides in length. More preferably, the mutation specific fluorescent probe is between 13 and 25 nucleotides in length.

In order to increase specificity, the nucleotide of the mutation specific fluorescent probe that corresponds to the mutated nucleotide in the RHOA nucleotide sequence may be present in substantially the center nucleotide of the mutation specific fluorescent probe sequence. The nucleotide of the mutation specific fluorescent probe that corresponds to the mutated nucleotide in the RHOA nucleotide sequence may be to located at a nucleotide that is within 20, 19, 18, 17, 16, 15, 14, 13, 12, 11, 10, 9, 8, 7, 6, 5, 4, 3, 2, or 1 nucleotides of the central nucleotide or nucleotides of the mutation specific fluorescent probe. Preferably, the nucleotide of the mutation specific fluorescent probe that corresponds to the mutated nucleotide in the RHOA nucleotide sequence is located at a nucleotide that is within 10 nucleotides of the central nucleotide or nucleotides of the mutation specific fluorescent probe. More preferably, the nucleotide of the mutation specific fluorescent that corresponds to the mutated nucleotide in the RHOA nucleotide sequence is located at a nucleotide that is within 5 nucleotides of the central nucleotide or nucleotides of the mutation specific fluorescent probe. Most preferably, the nucleotide of the mutation specific fluorescent probe that corresponds to the mutated nucleotide in the RHOA nucleotide sequence is located at a nucleotide that is within 1 nucleotides of the central nucleotide or nucleotides of the mutation specific fluorescent probe.

The reaction specificity may be increased by competitive binding. Thus, the method of the second aspect may further comprise adding to the mixture of step i) a fluorescent probe that is substantially complementary to the wild-type RHOA nucleotide sequence, herein defined “wild-type florescent probe”, and is labelled with a different fluorophore with emission spectrum non-overlapping with the mutation specific probe. The label may be 3′ or 5′ of the probe. Preferably the label is at the 5′ end.

Preferably, the wild-type fluorescent probe is dual labeled. Accordingly, the wild-type florescent probe may further comprise a fluorescence quencher, preferably at the 3′ end. Preferably the quencher is Black Hole-1 (BHQ-1). This advantageously enables simultaneous detection of the mutant and the wild-type signal from the same reaction well, by reading at the two emission lengths.

When the mutated RHOA nucleotide sequence results in the amino acid substitution F25L, the wild-type florescent probe preferably comprises a nucleic acid sequence substantially as set out in SEQ ID NO: 117, or a fragment or variant thereof.

When the mutated RHOA nucleotide sequence results in the amino acid substitution G17V, the wild-type florescent probe preferably comprises a nucleic acid sequence substantially as set out in SEQ ID NO: 119, or a fragment or variant thereof.

When the mutated RHOA nucleotide sequence results in the amino acid substitution C16Stop, the wild-type fluorescent probe preferably comprises a nucleic acid sequence substantially as set out in SEQ ID NO: 121, or a fragment or variant thereof.

When the mutated RHOA nucleotide sequence results in the amino acid substitution G14V, the wild-type fluorescent probe preferably comprises a nucleic acid sequence substantially as set out in SEQ ID NO: 123, or a fragment or variant thereof.

In a preferred embodiment, the wild-type florescent probe and/or the mutation specific fluorescent probe are synthesized with a modified base in correspondence of the variable position under investigation, to improve their affinity for the respective allele. In a more preferred embodiment the modified base is a locked nucleic acid (LNA). This strategy increases specificity and the overall Tm of the probe, enabling the design of shorter and more specific probes.

Each probe may contain more than one LNA, preferably spaced 2 or more nucleotides apart, in order to increase the probe Tm until the desired Tm value is obtained. The desired Tm value may be between 1° C. and 15° C., between 2° C. and 15 ° C., between 3° C. and 15° C., between 4° C. and 15° C., between 5° C. and 15° C., between 1° C. and 10° C., between 2° C. and 10 ° C., between 3° C. and 10° C., 4° C. and 10° C., between 5° C. and 10° C. above the wild-type florescent probe and/or the mutation specific florescent probe melting temperatures. Preferably, the desired Tm value is between 5° C. and 10° C. above the wild-type florescent probe and/or the mutation specific florescent probe melting temperatures. The Tm of both probes may be between 50° C. and 80° C., between 50° C. and 75° C., between 50° C. and 65° C., between 50° C. and 60° C., between 55° C. and 80° C., between 55° C. and 75° C., between 55° C. and 70° C., between 55° C. and 65° C., between 55° C. and 60° C., between 60° C. and 80° C. between 60° C. and 75° C., between 60° C. and 70° C.

Preferably, the Tm of either probe is between 60° C. and 70° C. Preferably, the Tm difference between the wild-type florescent probe and the mutation specific florescent probe is less than 10° C. More preferably less than 5° C., 4° C., 3° C., 2° C. or 1° C.

The probes may comprise one or more the following features:

5′ last nucleotide is not a G, nor is the penultimate (second-from-last) nucleotide;

3′ end does not have the sequences GGG or GGAG.

The presence of homopolymers (repeating nucleotides) is low. Preferably, no more than 4 Gs together, no more than 6 As together, and/or no more than 2 CC dinucleotides in the middle of the probe

In a different embodiment, the experiment setup can be modified to use a simple oligonucleotide with LNAs at the desired position instead of either the mutant or the wild-type fluorescent probe. In this embodiment, just the remaining probe may generate a fluorescent signal in case of hybridization and the LNA-oligonucleotide will serve to prompt competitive binding to increase specificity.

The method of the second aspect comprises the use of a primer set that is capable of hybridising to a wild-type RHOA encoding nucleotide target sequence. The primer set comprises a wild-type forward and wild-type reverse primer.

The wild-type primers may be capable of hybridizing to a different region of the RHOA nucleotide sequence than the mutation-specific forward primer and reverse primer are capable of hybridizing to. The wild-type primers are preferably capable of hybridizing to a region of the RHOA nucleotide sequence that flanks the mutation position of interest, without any overlap, thus targeting a region of the RHOA nucleotide sequence that does not comprise a mutation. The amplicon size may be between 50 and 120 nucleotides in length, preferably between 50 and 95 nucleotides in length. Preferably, the Tm of the forward and reverse primers is between 50° C. and 65° C., more preferably 30 between 55° C. and 60° C. The Tm difference between the forward and the reverse primers is preferably less than 5° C., more preferably less than 4° C., 3° C., 2° C. or 1° C.

Hence, when the mutated RHOA nucleotide sequence results in the amino acid substitution F25L, the wild-type forward primer may comprise a nucleic acid sequence as substantially set out in SEQ ID NO: 113, or a fragment or variant thereof, the wild-type reverse primer may comprise a nucleic acid sequence as substantially set out in SEQ ID NO: 114, or a fragment or variant thereof.

When the mutated RHOA nucleotide sequence results in the amino acid substitution G17V, C16Stop or G14V the wild-type forward primer may comprise a nucleic acid sequence as substantially set out in SEQ ID NO: 115, or a fragment or variant thereof, the wild-type reverse primer may comprise a nucleic acid sequence as substantially set out in SEQ ID NO: 116, or a fragment or variant thereof.

Preferably, the mutation specific fluorescent probe and the wild-type fluorescent probe whenever used have a Tm of between 5 and 10° C. above the wild-type forward and reverse primers Tm. For example the Tm of the mutation specific fluorescent probe and the wild-type fluorescent probe may be above 10° C. More preferably above 9° C., 8° C., 7° C., 6° C., 5° C., 4° C., 3° C., 2° C. or 1° C. the Tm of the wild-type forward and reverse primers Tm.

A reference assay can be designed and added to a separate reaction well, amplifying a second non-mutated target sequence on the RHOA nucleotide sequence, said second target sequence being located at a distance of between 100 and 400 nucleotides from the first target sequence on the RHOA nucleotide sequence, the first and second amplified sequences may be between 50 and 120 nucleotides in length, preferably between 50 and 95 nucleotides in length. Preferably the first and second amplified sequences have substantially the same length. The first and second amplified sequences may vary by less than 50 nucleotides in length, less than 40 nucleotides in length, less than 30 nucleotides in length, less than 20 nucleotides in length, less than to nucleotides in length, less than 5 nucleotides in length, less than 4 nucleotides in length, less than 3 nucleotides in length, less than 2 nucleotides in length or have the same number of nucleotides.

The method may be used to detect several different mutations in the RHOA gene from a subject sample, either sequentially, or in parallel.

When the mutated RHOA nucleotide sequence results in the amino acid substitution F25L, the forward primer may be substantially as set out in SEQ ID NO:113, or a fragment or variant thereof, the reverse primer substantially as set out in SEQ ID NO: 114, or a fragment or variant thereof, the mutation-specific probe substantially as set out in SEQ ID NO: 118, or a fragment or variant thereof and the wild-type specific probe substantially as set out in SEQ ID NO: 117, or a fragment or variant thereof.

When the mutated RHOA nucleotide sequence results in the amino acid substitution G17V, the forward primer may be substantially as set out in SEQ ID NO:115, or a fragment or variant thereof, the reverse primer substantially as set out in SEQ ID NO:116, or a fragment or variant thereof, the mutation-specific probe substantially as set out in SEQ ID NO: 120, or a fragment or variant thereof and the wild-type specific probe substantially as set out in SEQ ID NO: 119, or a fragment or variant thereof.

When the mutated RHOA nucleotide sequence results in the amino acid substitution C16Stop, the forward primer may be substantially as set out in SEQ ID NO:115, or a fragment or variant thereof, the reverse primer is substantially as set out in SEQ ID NO: 116, or a fragment or variant thereof, the mutation-specific probe substantially as set out in SEQ ID NO: 122, or a fragment or variant thereof and the wild-type specific probe substantially as set out in SEQ ID NO: 121, or a fragment or variant thereof.

When the mutated RHOA nucleotide sequence results in the amino acid substitution G14V, the forward primer may be substantially as set out in SEQ ID NO:115, or a fragment or variant thereof, the reverse primer substantially as set out in SEQ ID NO: 116, or a fragment or variant thereof, the mutation-specific probe substantially as set out in SEQ ID NO: 124, or a fragment or variant thereof and the wild-type specific probe substantially as set out in SEQ ID NO: 123, or a fragment or variant thereof.

Advantageously, by combining two primer sets, the method of the invention enables the determination of not only the presence or amount of a mutation, but also the level of total cfNA, thereby providing with high reliability a mutant allele frequency in circulating nucleic acids or other mixtures of nucleic acids.

Thus, in a third aspect of the invention there is provided a polymerase chain reaction method for determining the frequency of RHOA gene mutations in a sample obtained from a subject, the method comprising:
i) forming a mixture comprising the sample and a first primer set, wherein the sample comprises RHOA encoding nucleotide first and second target sequences and a primer set of the first aspect;
ii) (a) contacting the mixture of step i) with a second primer set comprising a wild-type forward primer and a wild-type reverse primer; or
   (b) forming a second mixture comprising the sample and a second primer set comprising a wild-type forward primer and a wild-type reverse primer;
iii) subjecting the mixture of step ii) (a) or the mixtures of step i) and ii) (b) to, sequentially in steps, a denaturing step, an annealing step, and an extension step, wherein in the annealing step the mutation-specific forward primer and the oligoblocker compete to anneal to the first target sequence, wherein the oligoblocker is capable of specifically hybridizing to a wild-type target sequence to block polymerase extension, and the reverse primer anneals to the second target sequence, wherein the second target sequence corresponds to a wild-type sequence of the RHOA gene; and
iv) obtaining at least one PCR product through steps (i) to (iii), wherein the presence of a first product amplified for the first target sequence resulting from the first primer set is indicative of the presence of a mutation in the RHOA gene in the sample and the presence of a second product amplified product from the second target sequence resulting from the second primer set is indicative of the total number of RHOA encoding nucleotide sequences in the sample;
v) comparing the amount of sequence amplified from the first and second target sequences to determine the frequency of RHOA gene mutations in a sample.

Step iii) may comprise obtaining a third PCR product that is produced by a combination of the first and second primer sets.

Preferably, the first primer set, mixture, RHOA nucleotide sequence and sample are as defined in the first aspect.

The comparing step (v) may comprise determining the ratio between the amount of sequence amplified from the first and second target sequences.

The skilled person would be aware of suitable PCR reagents and reaction conditions for successful amplification using either the mixtures of step ii) (a) or the mixtures of step i) and ii) (b).

The second target sequence may be located at a distance of about 10 to 1000, 20 to 1000, 30 to 1000, 40 to 1000, 50 to 1000, 60 to 1000, 70 to 1000, 80 to 1000, 90 to 1000, 100 to 1000, 10 to 900, 20 to 900, 30 to 900, 40 to 900, 50 to 900, 60 to 900, 70 to 900, 80 to 900, 90 to 900, 100 to 900, 10 to 800, 20 to 800, 30 to 800, 40 to 800, 50 to 800, 60 to 800, 70 to 800, 80 to 800, 90 to 800, 100 to 800, 10 to 700, 20 to 700, 30 to 7900, 40 to 700, 50 to 700, 60 to 700, 70 to 700, 80 to 700, 90 to 700, 100 to 700, 10 to 600, 20 to 600, 30 to 600, 40 to 600, 50 to 600, 60 to 600, 70 to 600, 80 to 600, 90 to 600, 100 to 600, 10 to 500, 20 to 500, 30 to 500, 40 to 500, 50 to 500, 60 to 500, 70 to 500, 80 to 500, 90 to 500, 100 to 500, 10 to 400, 20 to 400, 30 to 400, 40 to 400, 50 to 400, 60 to 400, 70 to 400, 80 to 400, 90 to 400, 100 to 400, 10 to 300, 20 to 300, 30 to 300, 40 to 300, 50 to 300, 60 to 300, 70 to 300, 80 to 300, 90 to 300 or 100 to 300 nucleotides from the first target sequence. Preferably, the second target sequence is located at a distance of about 100 to 400 nucleotides from the first target sequence.

The wild-type forward primer and wild-type reverse primer may be as defined in the second aspect.

The skilled person would understand that such a distance designates the number of nucleotides contained between the first amplified nucleotide of the first target sequence and the first amplified nucleotide of the second target sequence. Preferably, the distance is between 200 and 400 nucleotides, more preferably between 250 and 350 nucleotides. Most preferably, the distance is about 300 nucleotides.

The second amplified sequence may have substantially the same length as that of the first amplified sequence. The term “substantially the same” indicates that the two amplified sequences may not differ in length by more than 15% of total nucleotides, more preferably by not more than 10% of total nucleotides, and most preferably by not more than 5% of total nucleotides. For example if one set amplifies a sequence of 80 nt, the other set may amplify a sequence of between 68 and 92 nucleotides, preferably between 72 and 88 nucleotides, more preferably between 76 and 84 nucleotides.

Preferably, the first and second PCR products have a length of between 20 and 150, between 30 and 130, between 40 and 110, between 45 and 100, between 50 and 95 or 30 between 55 and 90 nucleotides. Preferably, the first and second PCR products have a length of between 50 and 95 nucleotides.

The third PCR product may be between 150 and 500, between 150 and 450, between 150 and 400, between 150 and 350, between 150 and 300, between 150 and 250, between 200 and 500, between 200 and 450, between 200 and 400, between 200 and 350, between 200 and 300 or between 200 and 250 nucleotides in length. Preferably, the third PCR product is between 200 and 400 nucleotides in length.

When the mutated RHOA nucleotide sequence results in the amino acid substitution F25L, and the first primer set may be as defined in the first aspect, and the second primer set may comprise a wild type primer substantially as set out in SEQ ID NO: 32 or a fragment or variant thereof and the second reverse primer that is substantially as set out in SEQ ID NO: 33 or a fragment or variant thereof; and/or

when the mutated RHOA nucleotide sequence results in the amino acid substitution G17V, and the first primer may be as defined in the first aspect, and the second primer set may comprise a wild-type primer substantially as set out in SEQ ID NO: 70 or a fragment or variant thereof and a second reverse primer substantially as set out in SEQ ID NO: 71 or a fragment or variant thereof; and/or

when the mutated RHOA nucleotide sequence results in the amino acid substitution C16Stop, and the first primer may be as defined in the first aspect, and the second primer set may comprise a wild-type primer substantially as set out in SEQ ID NO: 70 or a fragment or variant thereof and a second reverse primer substantially as set out in SEQ ID NO: 71 or a fragment or variant thereof; and/or

when the mutated RHOA nucleotide sequence results in the amino acid substitution G14V, and the first primer set may be as defined in the first aspect, and the second set of reagents may comprise a wild-type primer substantially as set out in SEQ ID NO: 70 or a fragment or variant thereof and a second reverse primer substantially as set out in SEQ ID NO: 71 or a fragment or variant thereof.

Advantageously, the detection of these mutations, alone or in combination, enables reliable diagnosis or prognosis of cancer in a subject.

Accordingly, in a fourth aspect of the invention there is provided a method of diagnosing cancer in a subject, or a predisposition thereto, or for providing a prognosis of cancer, comprising:
a) i) performing a polymerase chain reaction (PCR) method according to the first aspect; and
   ii) detecting the presence of PCR products obtained through step (i) to diagnose or prognose cancer in the subject, wherein the presence of a PCR product is indicative of cancer;
b) i) performing a PCR method according to the second aspect; and
   ii) detecting the presence of a fluorescent signal obtained by the reaction of step i) to diagnose or prognose cancer in the subject, wherein the presence of a fluorescent signal is indicative of cancer; or
c) i) performing a PCR method according to the third aspect; and
   ii) comparing the amount of sequence amplified from the first and second target sequences to determine the frequency of RHOA gene mutations in a sample thereby diagnosing or prognosing cancer in the subject.

Preferably, the cancer is peripheral T-cell lymphoma.

In a fifth aspect of the invention, there is provided a method of treating cancer in a subject:
a) i) performing a polymerase chain reaction (PCR) method according to the first aspect;
   ii) detecting the presence of a PCR product that obtained through step (i), wherein the presence of the PCR product is indicative of the presence of a mutation in the RHOA gene; and
   iii) administering, or having administered, an effective amount of an anti-cancer composition to the subject thereby treating the patient;
b) i) performing a PCR method according to the second aspect; and
   ii) detecting a fluorescent signal obtained by the reaction of step i), wherein the presence of a fluorescent signal is indicative of the presence of a mutation in the RHOA gene; and
   iii) administering, or having administered, an effective amount of an anti-cancer composition to the subject thereby treating the patient; or
c) i) performing a PCR method according to the third aspect; and
   ii) comparing the amount of sequence amplified from the first and second target sequences to determine the frequency of RHOA gene mutations in a sample that is associated with cancer; and
   iii) administering, or having administered, an effective amount of an anti-cancer composition to the subject thereby treating the patient.

Preferably, the cancer is peripheral T-cell lymphoma.

The invention also extends to the primers and probes that are utilised in the methods of the invention.

Accordingly, in the sixth aspect of the invention there is provided a mutation-specific primer sequence as defined in the first aspect.

The primers of the sixth aspect are preferably used in combination with a reverse primer, which hybridizes to a wild type sequence of the RHOA nucleic acid sequence i.e. a portion of the nucleic acid sequence that is non-mutated, and amplifies, in combination with a primer sequence of the first aspect, a target RHOA sequence comprising a mutation.

Accordingly, in a seventh aspect there is provided a mutation-specific forward primer and reverse primer pair as defined in the first aspect.

In an eighth aspect there is provided a primer set as defined in the first aspect.

In a ninth aspect there is provided a mutation specific fluorescent probe as defined in the second aspect.

In a tenth aspect there is provided a mutation specific fluorescent probe and wild-type specific fluorescent probe pair as defined in the second aspect.

In a eleventh aspect there is provided the primer of the sixth aspect, the primer pair of the seventh aspect, the primer set of the eighth aspect, the mutation specific fluorescent probe of the ninth aspect or the probe pair of the tenth aspect for use in in vivo diagnosis.

In a twelfth aspect there is provided the primer of the sixth aspect, the primer pair of the seventh aspect, the primer set of the eighth aspect, the mutation specific fluorescent probe of the ninth aspect or the probe pair of the tenth aspect for use in vivo diagnosis of cancer.

Preferably the cancer is peripheral T-cell lymphoma.

In a thirteenth aspect there is provided a kit for determining the presence or frequency of a mutation in the RHOA gene, comprising:
a) i) at least one primer of the sixth aspect, at least one set of primers as defined in the seventh aspect; at least one primer set of the eighth aspect; or
   ii) at least one mutation specific fluorescent probe of the ninth aspect, or at least one probe pair of the tenth aspect;
b) a DNA polymerase
c) optionally a sample obtained from a subject; and
d) instructions for use.

The sample may be as defined in the first aspect.

The kit may further comprise polynucleotides and/or a PCR reaction buffer.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-180 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different to values depending on: (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage of identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage of identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula: Sequence Identity=(N/T)*100.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

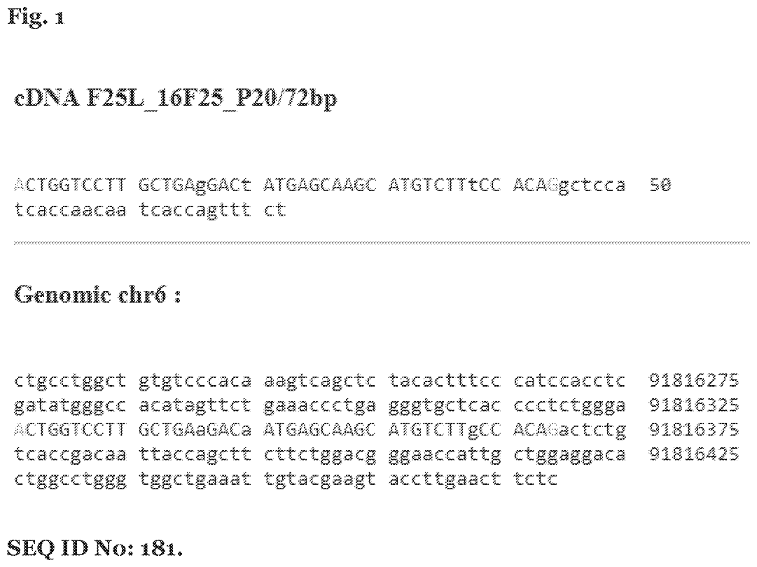

FIG. 1 shows Blat alignments (Human GRCh38/hg38) for all the PCR amplicons generated by the combinations of primers meeting the thermodynamic criteria

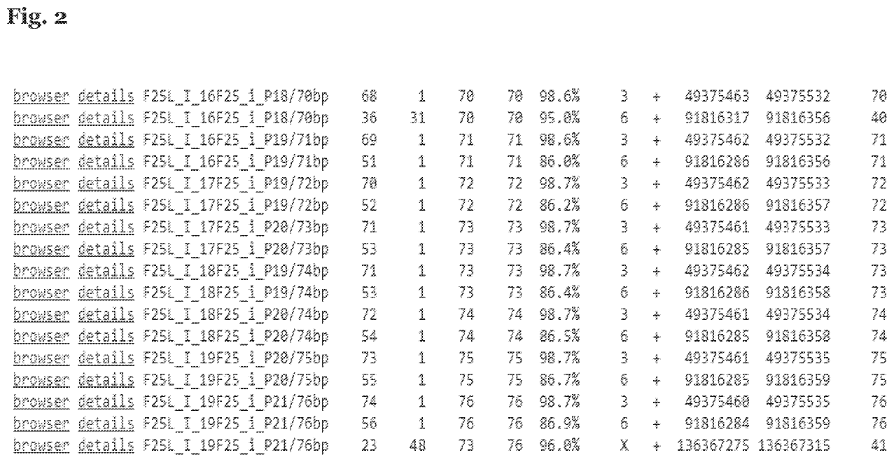

FIG. 2 shows a list of partial homologies generated by the proposed amplicons in inverse configuration

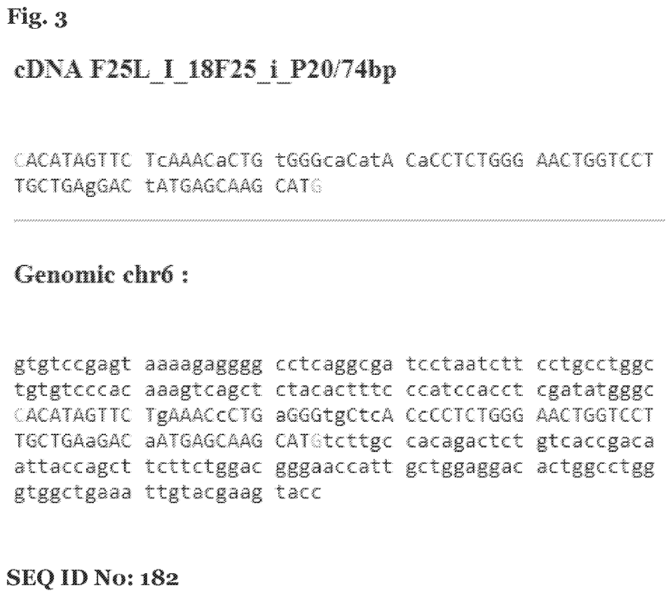

FIG. 3 shows detailed examples of the homologies generated by the proposed amplicons in inverse configuration.

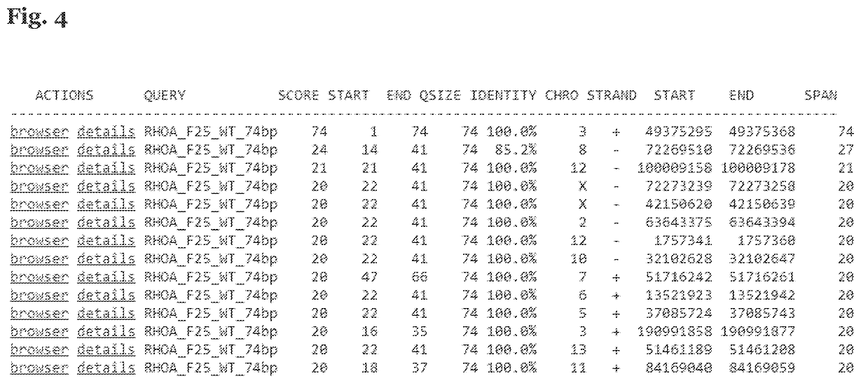

FIG. 4 shows blat alignments (Human GRCh38/hg38) for the PCR amplicons generated by the combinations of primers meeting the IntPlex® thermodynamic criteria.

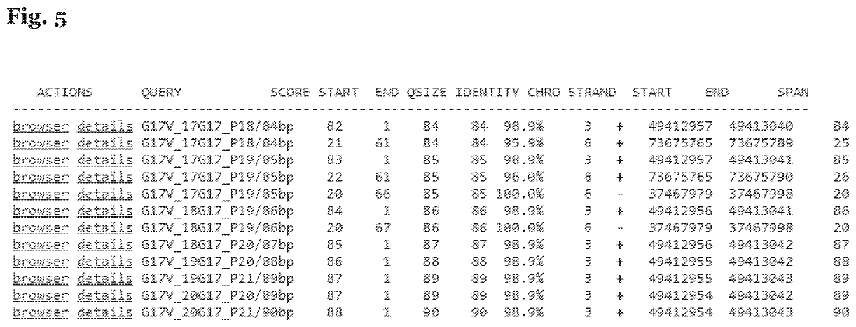

FIG. 5 shows BLAT analysis results for all the expected PCR products generated with the proposed primer sets in conventional configuration for the detection of the RHOA G17V mutation.

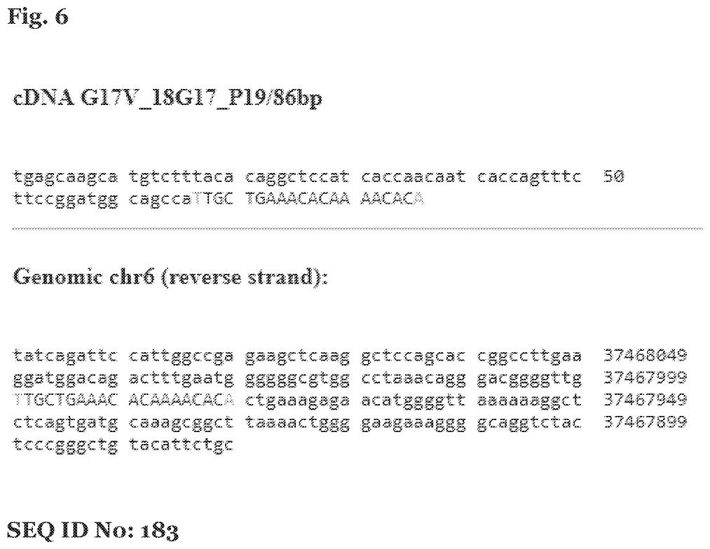

FIG. 6 shows Blat alignments (Human GRCh38/hg38) for all the PCR amplicons generated by the combinations of primers meeting the thermodynamic criteria.

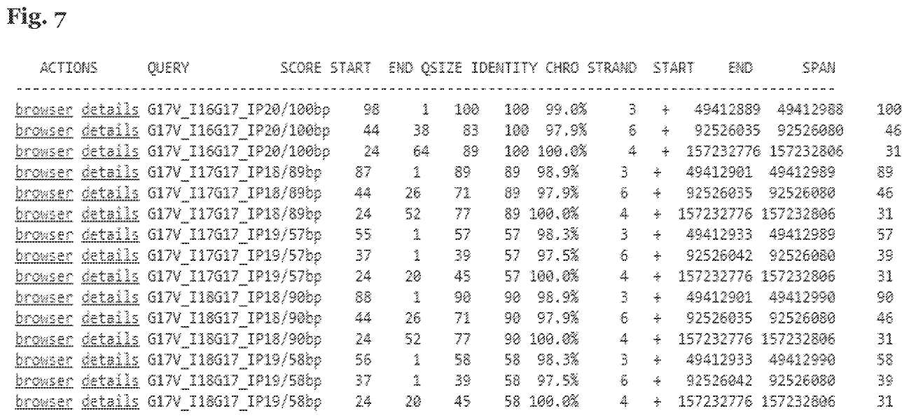

FIG. 7 shows a list of partial homologies generated by the proposed amplicons in inverse configuration.

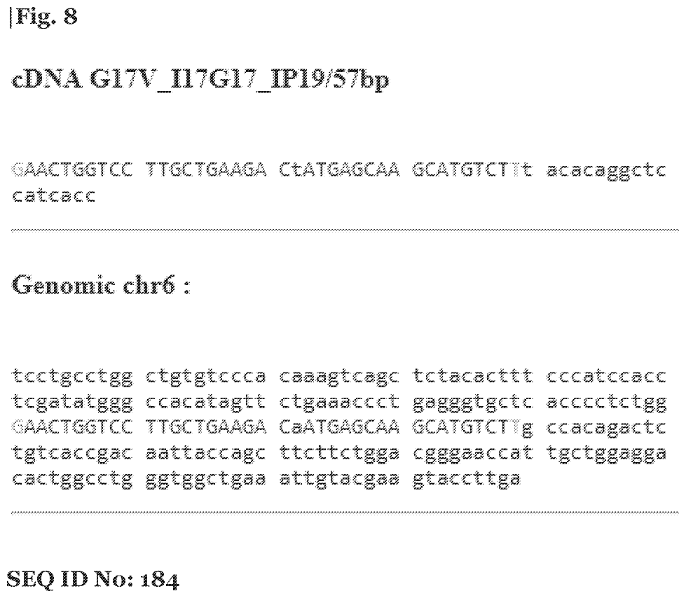

FIG. 8 shows detailed examples of the homologies generated by the proposed amplicons in inverse configuration.

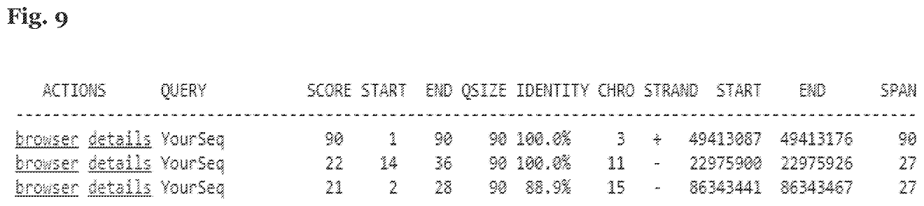

FIG. 9 shows Blat alignments (Human GRCh38/hg38) for the PCR amplicons generated by the combinations of primers meeting the IntPlex® thermodynamic criteria.

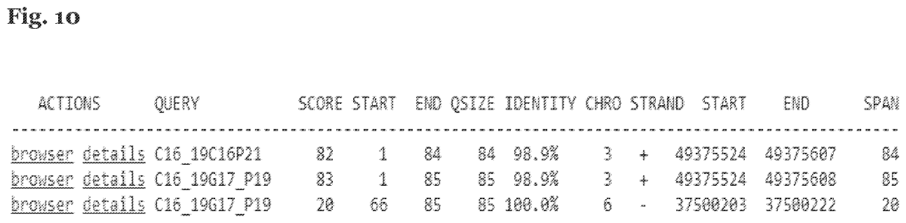

FIG. 10 shows BLAT analysis results for all the expected PCR products generated with the proposed primer sets in conventional configuration for the detection of the RHOA C16* mutation.

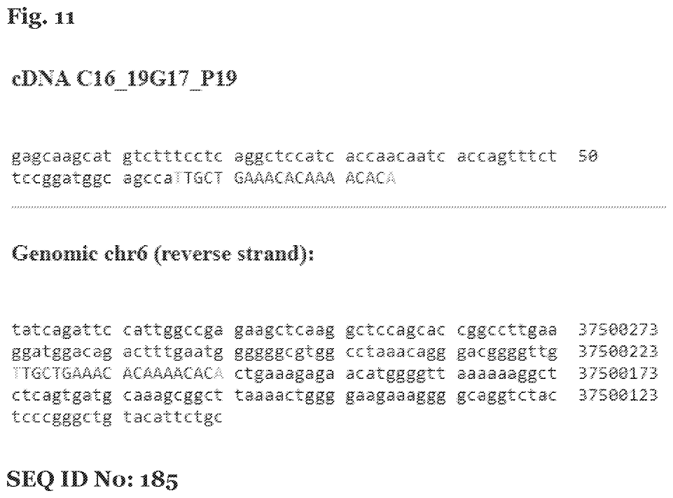

FIG. 11 shows Blat alignments (Human GRCh38/hg38) for all the PCR amplicons generated by the combinations of primers meeting the thermodynamic criteria.

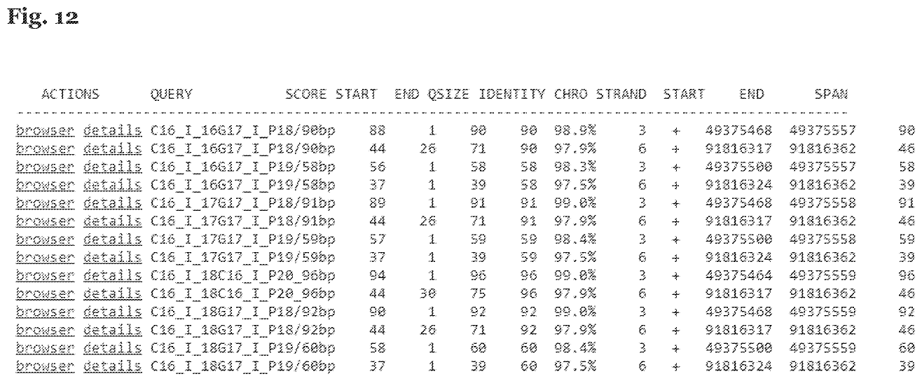

FIG. 12 shows a list of partial homologies generated by the proposed amplicons in inverse configuration.

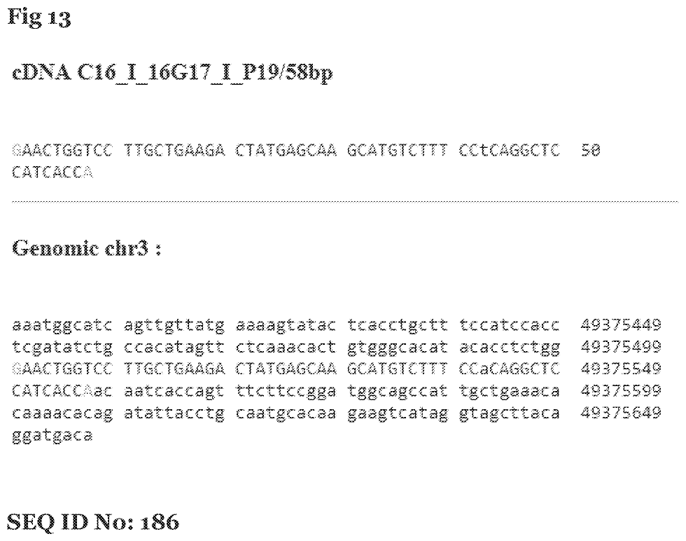

FIG. 13 shows detailed examples of the homologies generated by the proposed amplicons in inverse configuration.

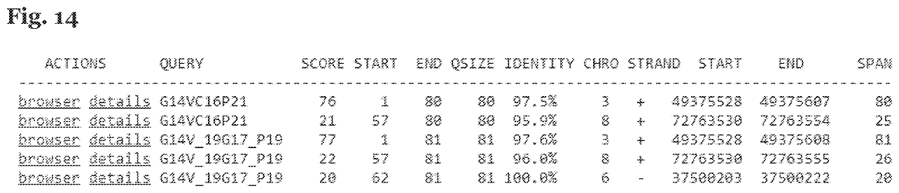

FIG. 14 shows BLAT analysis results for all the expected PCR products generated with the proposed primer sets in conventional configuration for the detection of the RHOA G14V mutation.

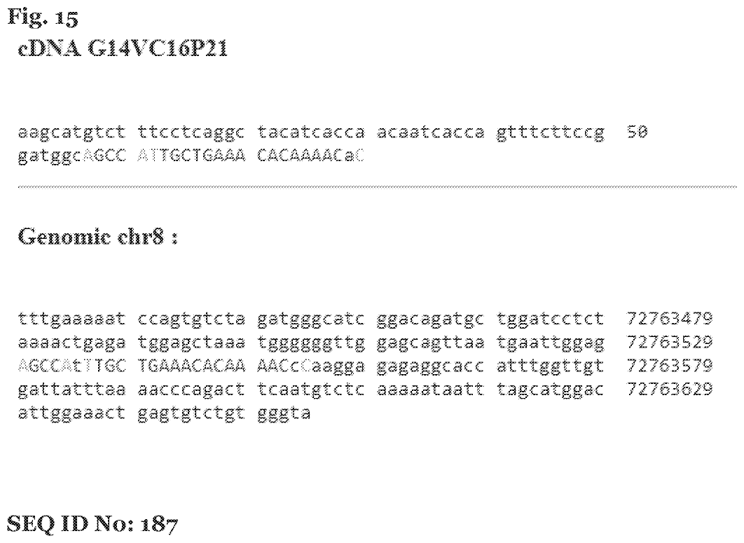

FIG. 15 shows Blat alignments (Human GRCh38/hg38) for all the PCR amplicons generated by the combinations of primers meeting the thermodynamic criteria.

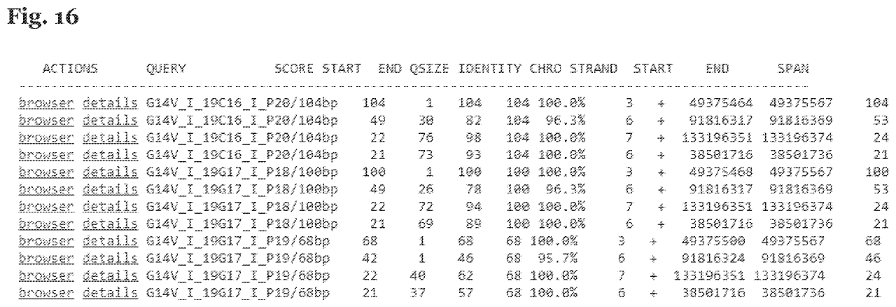

FIG. 16 shows a list of partial homologies generated by the proposed amplicons in inverse configuration.

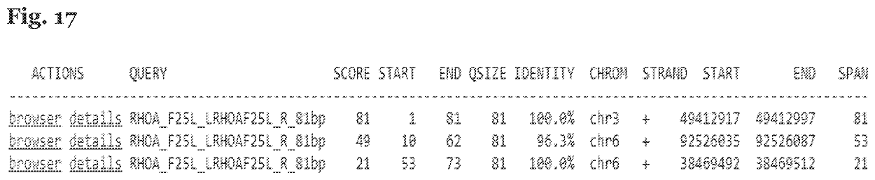

FIG. 17 shows BLAT analysis results for the expected PCR products generated with the proposed primer sets for the detection of the RHOA F25L mutation.

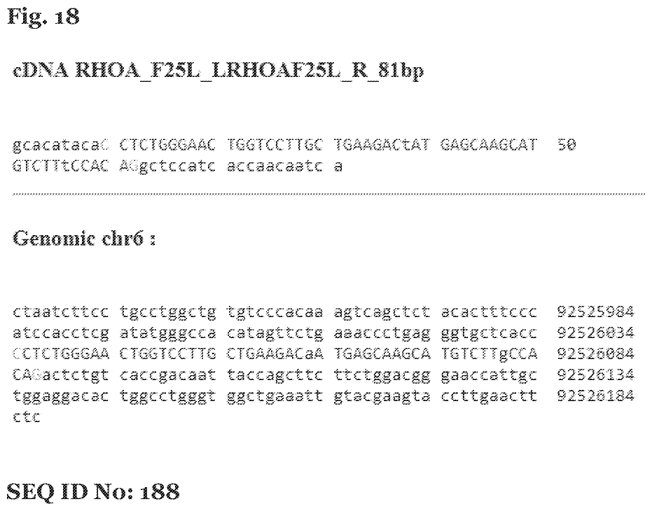

FIG. 18 shows Blat alignments (Human GRCh38/hg38) for all the PCR amplicons generated by the combinations of primers meeting the thermodynamic criteria.

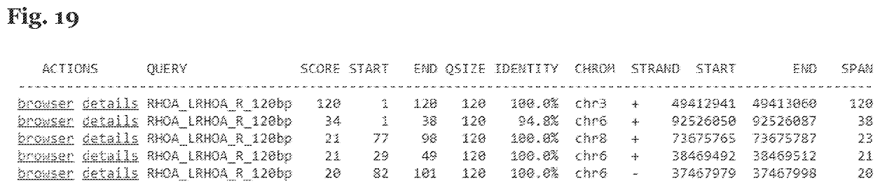

FIG. 19 shows BLAT analysis results for the expected PCR products generated with the proposed primer sets for the detection of the G17V mutation.

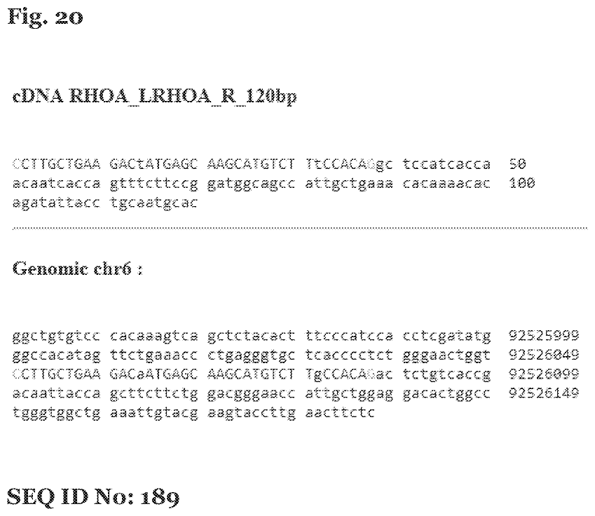

FIG. 20 shows Blat alignments (Human GRCh38/hg38) for all the PCR amplicons generated by the combinations of primers meeting the thermodynamic criteria.

### EXAMPLES

Materials and Methods

Design of the Allele-Specific Primer Targeting the Mutation of Interest

To optimise specificity and sensitivity of the allele-specific primer targeting the single nucleotide mutation of interest, the mutant base was generally positioned as 3′ end last nucleotide. The 3′ end of an allele-specific primer is mismatched for one allele (the WT allele) and is a perfect match for the other allele (the mutated allele). The 3′ positioning of the mismatch has the effect of partially blocking the amplification of the WT allele. Indeed, the Taq polymerase enzyme typically used for DNA amplification preferably extends DNA in presence of a perfect match at the 3′ primer end with a free hydroxyl group present. Furthermore, any residual non-specific amplification from the WT allele is further avoided using an oligoblocker.

The positioning of the targeted mutation as the 3′ end of the allele-specific primers generates two possible options for assay design: one in “conventional configuration”, with the primer sequence matching the reference sequence on the positive strand (hence with the primer annealing to the negative strand) with the mutation in 3′; one in “inverse configuration”, with the primer sequence matching the negative strand (hence with the primer annealing to the positive strand) with the mutation in 3′.

Both options were considered in the experimental section, and the thermodynamic characteristics of both options were generally compared, to ensure optimal Tm and GC % with absence of primer-dimers, blocker-primer dimers, self-dimerisation, and hairpins.

In addition, different combinations of primers at different lengths were tested, to provide optimal results.

In the selection of the primers targeting the mutation of interest the following recommendations were followed to prevent the risk of non-specific amplification and to improve the method's sensitivity:

Low melting temperature (Low Tm): Ideally, the Tm of the primer should be 10° C. below the annealing temperature of the PCR system (60° C.), with accepted range between 45-55° C. As the variant is targeted at the 3′end last base of the primer, Tm changes towards the optimal temperature are obtained by varying the primer length at the 5′ end.

Length of the allele-specific primer: The primer targeting the mutation should have low Tm (45-55° C.), suggesting a short length but it should be long enough to be highly specific and provide efficient amplification. Length was found optimal between 16 and 22 bp.

GC %: The GC content (percentage of guanines and cytosines out of the total number of bases) of the primer should ideally fall between 40 and 60%. Lower the GC %, lower the risk of non-specific amplification but it is associated with lower amplification efficiency. Higher the GC %, higher the risk of non-specific amplification but the amplification efficiency is increased.

3′-tail GC %: Higher values (>25%) allow more specific annealing of the primer to its target sequence.

Secondary structures: the formation of primer self-dimers or hairpin loops is detrimental to the PCR efficiency as it considerably decreases the concentration of primer molecules available for the PCR reaction. Also, secondary structures can generate strong background signals that can negatively impact on the quantification accuracy of the desired PCR product. The software Oligoanalyzer® was used to analyse the change in Gibbs free energy required for breaking down the predicted secondary structures (←G). pG below −4 kcal/mol are indicative of primers at a very strong risk of self-annealing that should hence be modified to reach ΔG values closer to zero or discarded. Absence of any hairpin is ideal. In presence of hairpins with ΔG below −3 kcal/mol the primer should be modified to reach ΔG values closer to zero or discarded.

Paired Wild-Type Primer to the Allele-Specific Primer

In the selection of the paired wild-type primer the following criteria were followed to prevent the risk of non-specific amplification and to improve the method's sensitivity:

Amplicon size: the paired WT primer was positioned at 60-100 bp from the primer targeting the mutation in order to produce an amplicon of 60-100 bp. Such size was indeed found optimal by the inventors for detecting tumour-derived circulating DNA.

In the conventional configuration (allele-specific primer designed upon the reference sequence, annealing to the negative strand), the paired wild-type primer sequence is the complement-reverse of the reference sequence, in order to anneal to the positive strand.

In the inverse configuration (allele-specific primer designed as complement-reverse of the reference sequence to maintain the mutation in 3′, annealing to the positive strand), the paired wild type sequence is designed upon the reference sequence, in order to anneal to the negative strand.

Melting temperature: Ideally, the Tm difference between the Tm of the primer targeting the mutation and its paired WT primer should not exceed 5° C. As a consequence, the Tm of the paired WT primer should preferably be 50-60° C.

GC %, 3′ tail GC % and secondary structures: the same parameters described under “Design of the allele-specific primer targeting the mutation of interest”.

Off-target amplification: Co-amplification of any secondary product other than the target sequence should be avoided to ensure specificity. Presence of off-target amplification would generate false negative results and compromise the validity of the proposed method. To prevent this eventuality, the selected WT paired primers, together with their matched allele-specific primers, were analysed using several methods to confirm specificity for optimal primer combinations.

Unwanted homology regions: The amplicons are typically analysed using the “Blat” online tool on the UCSC Genome Browser website to determine the presence of potential secondary regions of homology that might generate non-specific products or alter the PCR efficiency.

Oligoblocker Design

The oligoblocker is an oligonucleotide complementary to the WT allele with respect to the mutation of interest. It is modified (e.g., phosphorylated in 3′) to block the polymerase elongation. The role of the oligoblocker is to avoid non-specific amplification of the WT sequence at the mutation locus. When the oligoblocker is hybridised to the WT sequence it avoids non-specific hybridisation of the primer targeting the mutation to the WT sequence, as it partially overlaps the primer target sequence.

The criteria for an optimal oligoblocker design are herein described, to prevent the risk of non-specific amplification and to improve the detection of low-frequency mutations.

Melting temperature: The oligoblocker Tm should preferably be close to 60° C. (the hybridization/extension temperature of the polymerase enzyme required for the PCR amplification reaction) and preferably at least 4° C. above the Tm of the primer targeting to the mutation. This preferred strategy ensures that the oligoblocker hybridizes to its target sequence before any potential non-specific hybridization to the WT locus by the allele-specific primer. Ideally, the oligoblocker Tm should hence be 55-60° C.

Position: The oligoblocker should be designed preferably so as to have the variant nt position towards the middle of its sequence. With this strategy, the oligoblocker occupies a larger portion of the allele-specific primer target region, aiding to avoid non-specific hybridization.

GC % and 3′GC %: the same parameters as described under “Design of the allele-specific primer targeting the mutation of interest” were followed.

Secondary structures: The same parameters as described under “Design of the allele-specific primer targeting the mutation of interest” were followed.

Length: The ideal length of an oligoblocker is between 19-23 bp.

Combinatorial Hetero-Dimer Analysis

The absence of hetero-annealing among the oligonucleotides (allele-specific primer, paired WT primer and oligoblocker) should preferably be verified. This strategy prevents the risk of forming primer-dimers and non-specific amplification, improving the efficiency of the method. The software Oligoanalyzer® can be used for this purpose and, based on its algorithms for the calculations of Gibbs free energy, just values above −4 Kcal/mol can be accepted. Oligonucleotide combinations with ΔG←4 kcal/mol need to be discarded and re-designed.

Design of the Wild-Type Primer Pair

This primer set should target a nucleic acid sequence located 100-350 bp, typically 300±10 bases pair, from the nucleic acid sequence targeted by the allele-specific primer set. Moreover, it should preferably target a nucleic acid sequence of the same size than the one targeted by the allele-specific primer set ±10%. Preferred characteristics of the primers that ensure optimal performance are listed below:

Melting temperature: The melting temperature should be comprised between 55 and 60° C., with less than 5° C. difference between the Tm of the two primers.

All other thermodynamic parameters to consider in the design of the wild-type primer pair (GC %, 3′GC %, amplicon size, secondary structures, off-target amplification and unwanted homology regions) are the same as described under “Paired Wild-Type primer to the allele-specific primer”.

In Vitro Validation for Mutated Region

Specificity test: This phase is generally performed for verifying that the mutation-specific primer set under evaluation amplifies only its target sequence. It is typically realised on genomic DNA carrying the mutation of interest or from synthetic double-strand DNA fragments carrying the mutation of interest diluted in wild-type genomic DNA background.

Non-specificity test: The non-specificity test can be performed to ensure that the primer set targeting the mutation of interest does not amplify any other region and that no primers-dimers or self-dimers are formed during the PCR reaction. Criteria to validate the non-specificity are typically: concentration values below 0.5 pg/μl and a different Tm from the expected Tm of the mutation, as determined by the specificity test.

Efficiency test: The efficiency of mutation-specific primer set is generally evaluated by running the assay on positive controls of known mutational load.

Sensitivity: To assess the sensitivity of the system in analysis, an assay is generally run on serial dilutions of DNA mutant for the variant in analysis.

Considerations for Probe-Based A-TAG Assays

Some extra considerations should be applied when adapting the A-TAG assay to a probe-based detection system.

Primer Design

The primer melting temperature should be comprised between 55° C. and 60° C. The primers should flank the mutant site with no overlapping allowed with the variant site in analysis.

All other thermodynamic considerations presented above regarding primer length, GC %, 3′ GC tail and secondary structure are applicable.

All thermodynamic considerations presented above regarding the design of the paired primer, the maximum temperature difference between primer pairs, their respective orientation and the analysis of off-target amplification and presence of unwanted homology regions are applicable.

Oligoblocker

No oligoblocker is required in this experimental setting. The wild-type allele and the mutant allele will be detected in the same reaction well.

Probe Design

The mutation of interest is targeted by the mutant probe, labelled in 5′ with a suitable fluorophore group, as 6FAM, HEX or VIC and with a suitable quencher at the 3′, as Black Hole-1 (BHQ-1). The wild-type allele is targeted by the wild-type probe, labelled in 5′ with a different fluorophore with emission spectrum non-overlapping with the mutant probe and with a suitable quencher at the 3′, as Black Hole-1 (BHQ-1). This strategy enables simultaneous detection of the mutant and the wild-type signal from the same reaction well, by reading at the two emission lengths.

Probe length, 13-25 nucleotides, with optimum Tm 5-10° C. above the primers Tm. Shorter probes are favoured as they tend to have better specificity.

Position of variant in analysis: In order to increase specificity, in case of a single nucleotide mutation, the site of the nucleotide targeted by the mutant probe and by the wild-type probe shall be preferably centered on the probe sequence, more preferably located within 80% central nucleotides, even more preferably within 60% central nucleotides, even further preferably within 40% central nucleotides.

Use of LNAs: The wild-type probe and the mutant probe are synthesised with a locked nucleic acid in correspondence of the variable position under investigation, to improve their affinity for the respective allele. This strategy increases specificity and the overall Tm of the probe, enabling the design of shorter and more specific probes. Each LNA increases the probe Tm of 2° C.

When required by thermodynamic constraints, each probe can contain more than one LNA, ideally spaced of 2 or more base pairs, in order to increase the probe Tm until the desired value.

Alternative setup: if desired, or if working in one fluorescent channel only, the experiment setup can be modified to use a simple oligonucleotide with LNAs at the desired position instead of either the mutant or the wild-type probe. In this setup, just the remaining probe will generate a fluorescent signal in case of hybridization and the LNA-oligonucleotide will serve to prompt competitive binding to increase specificity. No detectable signal will be generated from the LNA oligonucleotide.

Sequence constraints on probe design. The following recommendations should be followed whenever possible, to ensure optimal fluorescence levels:
The probe may be designed within the sequence amplified by the selected primers flanking the variant position of interest.
5′ last nucleotide may not be a G; neither may the penultimate (second-from-last) nucleotide.
3′ end may have few Gs; specifically avoid GGG and GGAG
Keep homopolymers (repeating nucleotides) to a minimum, preferably no more than 4 Gs together, no more than 6 As together, no more than 2 CC dinucleotides in the 30 middle of the probe.
The probe may have <30 nucleotides between the fluorophore and the quencher to avoid affecting baseline signal intensity.
The sequence within the target may have a GC content of 30-80%
The probe may anneal to the strand that has more Gs than Cs (so the probe contains more Cs than Gs).

Combinatorial Heterodimer Analysis

The absence of hetero-annealing among the oligonucleotides (forward primer, paired reverse primer, wild-type probe and mutant probe) should preferably be verified. This strategy prevents the risk of forming primer-dimers and non-specific amplification, improving the efficiency of the method. The software Oligoanalyzer® can be used for this purpose and, based on its algorithms for the calculations of Gibbs free energy, just values above −4 Kcal/mol can be accepted. Oligonucleotide combinations with □G←4 kcal/mol need to be discarded and re-designed.

In Vitro Validation

All in vitro validation phases presented below should be thoroughly followed. A successful specificity test for a probe-based A-TAG assay will generate signal just from the wild-type probe. No amplification should be detected in the mutant probe channel when analysing wild-type DNA.

RHOA F25L

The inventors have designed specific sets of primers allowing improved detection of the presence (or absence, or frequency) of such a mutation from a sample containing cfNA or genomic DNA.

More specifically, in a preferred embodiment, the mutation is RHOA F25L, where the phenylalanine to leucine change is due to a single point mutation at position chr3: 49412950 (human assembly GRCh37/hg19) where the wild-type allele is an adenine (A) and the mutant allele is a guanine (G). The first set of reagents for detecting the RHOA F25L mutation comprises a mutation-specific primer selected from SEQ ID NO: 1-14, a paired primer selected from SEQ ID NO: 15-20, and an oligoblocker selected from SEQ ID NO:21-31, 34-38. Most preferably, the mutation-specific primer is selected from SEQ ID NO: 5-7, 11-16, the paired primer is selected from SEQ ID NO: 15, 17-20 and the oligoblocker is selected from SEQ ID NO: 23-24, 36-37. In another embodiment, a different paired primer can be chosen from ANY consecutive DNA sequence of size comprised between 15 and 25 base pairs selected in the following regions: chr3: 49412715-49412925 or chr3: 49412975-4913185 (human assembly GRCh37/hg19), according to the orientation of the allele-specific primer. A different oligoblocker can be chosen from ANY consecutive DNA sequence of size comprised between 15 and 25 base pairs selected in the following regions: chr3: 49412925-49412975 (human assembly GRCh37/hg19) with the fundamental prerequisite that the position chr3: 49412950 must be included in the selected oligoblocker sequence. In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 32 and a second primer SEQ ID NO: 33.

Primer Design for the Allele-Specific RHOA F25L IntPlex® System

The allele-specific primers considered in the design of this invention and their thermodynamic characteristics are listed in Table 2:

TABLE 2
List of all the RHOA F25L allele-specific primers considered in the design of
this invention and their thermodynamic characteristics. Accepted values are presented
with white background. Borderline values are presented with light grey background and
non-acceptable values are presented with dark grey background. In the eventuality that
one of the parameters in analysis falls outside the accepted range indicated in the last
row the thermodynamic analysis is discontinued and the primer is discarded.

| | | | | | | | |
|---|---|---|---|---|---|---|---|
| Primer name | Sequence | Length | Tm (° C.) | GC% | GC% tail | self-dimers | Hairpins |

Conventional configuration

| | | | | | | | |
|---|---|---|---|---|---|---|---|
| F25L 22 | CTGGGAACTGGTCCTTGCTGAG | 22 | 70 | | | | |
| F25L 21 | TGGGAACTGGTCCTTGCTGAG | 21 | 66 | | | | |
| F25L 20 | GGGAACTGGTCCTTGCTGAG | 20 | 64 | | | | |
| F25L 19 | GGAACTGGTCCTTGCTGAG | 19 | 60 | | | | |
| F25L 18 | GAACTGGTCCTTGCTGAG | 18 | 56 | 55.6 | 57.1 | none | none |
| F25L 17 | AACTGGTCCTTGCTGAG | 17 | 52 | 52.9 | 57.1 | none | none |
| F25L 16 | ACTGGTCCTTGCTGAG | 16 | 50 | 56.3 | 57.1 | none | none |

Inverse configuration

| | | | | | | | |
|---|---|---|---|---|---|---|---|
| F25L\|22 | AAGACATGCTTGCTCATAGTCC | 22 | 64 | | | | |
| F25L\|21 | AGACATGCTTGCTCATAGTCC | 21 | 62 | | | | |
| F25L\|20 | GACATGCTTGCTCATAGTCC | 20 | 60 | | | | |
| F25L\|19 | ACATGCTTGCTCATAGTCC | 19 | 56 | 47.4 | 42.9 | -2.17 | -0.1 |
| F25L\|18 | CATGCTTGCTCATAGTCC | 18 | 54 | 50 | 42.9 | -2.17 | -0.1 |
| F25L\|17 | ATGCTTGCTCATAGTCC | 17 | 50 | 47.1 | 42.9 | -0.22 | -0.1 |
| F25L\|16 | TGCTTGCTCATAGTCC | 16 | 48 | 50 | 42.9 | none | none |

| | | | | | | |
|---|---|---|---|---|---|---|
| Accepted values for the parameters | 16-22 | 45-55° | 40-60% | >25% | >-4 Kcal/ | >-3 Kcal/ |
| in analysis (allele-specific primer) | bp | C. | | | mol | mol |

Paired Wild-Type Primer

Results obtained with Primer 3

All allele-specific primers that met IntPlex® criteria (see Table 2) were used on Primer3 to find a compatible paired primer. Results are summarised in Table 3.

TABLE 3
List of all the RHOA F25L paired WT primers produced by Primer 3 and
considered in the design of this invention and their thermodynamic characteristics.

| Allele- | Allele-specific | Paired WT | | | | | | | | | | Off target |
|---|---|---|---|---|---|---|---|---|---|---|---|---|
| specific | Primer Tm | primer | Paired | | Amplicon | Tm | | GC% | self- | | Off | with wt allele- |
| Primer | (Primer3) | name | WT primer sequence | Length | size | (° C.) | GC% | tail | dimers | Hairpins | target? | specific? |
| F25L 18 | 54.95 | F25 P20 | AGAAACTGGTGATTGTTGGT | 20 | 74 | 55.07 | 40 | 42.9 | -0.07 | 0.04 | no | no |
| F25L 17 | 53.35 | F25 P20 | AGAAACTGGTGATTGTTGGT | 20 | 73 | 55.07 | 40 | 42.9 | -0.07 | 0.04 | no | no |
| F25L 16 | 52.18 | F25 P20 | AGAAACTGGTGATTGTTGGT | 20 | 72 | 55.07 | 40 | 42.9 | -0.07 | 0.04 | no | no |
| F25L 19 | 54.96 | F25 \| P20 P3 | CCTCGATATCTGCCACATAG | 20 | 88 | 54.93 | 50 | 42.9 | -3.86 | | | |

| | | | | | | | | | |
|---|---|---|---|---|---|---|---|---|---|
| Accepted values for the parameters in analysis | 16-25 | <100 bp | ΔTm with | 10-60% | >25% | >-4 Kcal/ | >-3 Kcal/ | no | no |
| (paired WT primer) | bp | | paired | | | mol | mol | | |
| | | | primer | | | | | | |
| | | | <5° C. | | | | | | |

Note that Tm are calculated following the recommended Primer3 algorithm. Accepted values are presented with white background and non-acceptable values are presented with dark grey background. In the eventuality that one of the parameters in analysis falls outside the accepted range indicated in the last row the thermodynamic analysis is discontinued and the primer is discarded.

From the results presented in Table 2 it was possible to select a definite candidate as paired WT primer for the RHOA F25L allele-specific primers F25L 18, F25L 17 and F25L 16 in conventional configuration.

On the other hand, the only potential paired wt-primer according to the allele-specific primers in inverse configuration F25L I 19, F25L I 18, F25L I 17, F25L I 16 contains a region of self-dimerization outside the accepted range.

The paired-wild type primer in inverse configuration may be manually designed following the thermodynamic criteria described in section 3.4.2 (common part of the patent file) and avoiding the self-dimerization region.

The paired WT primer F25 P20 in conventional configuration is suitable for in vitro evaluation, in combination with the allele-specific primers: F25L I 19, F25L I 18, F25L I 17, F25L I 16.

Region triggering high self-dymerisation in inverse configuration

SEQ ID No: 135
TTCCATCCACCTCGATATCTGCCACATAGTTCTCAAACACTGTGGGCAC
ATACACCTCTGGGAACTGGTCCTTGCTGAGGACTATGAGCAAGCATGTC
TTTCGATATC

Region triggering self-annealing as predicted by the In Silica software.

SEQ ID No: 136
GGACTATGAGCAAGCATGTCTT

—position of the allele-specific primer in inverse configuration.

Manually generated primer pair candidates in inverse configuration

TABLE 4
List of all potential RHOA F25 paired WT-primers in conventional
configuration manually generated and considered in the design of this invention and
their thermodynamic characteristics. Note that Tm are calculated using the
Oligoanalyzer ® software. Accepted values are presented with white background and
borderline values are presented with light grey background. In the eventuality that one
of the parameters in analysis falls outside the accepted range indicated in the last row
the thermodynamic analysis is discontinued and the primer is discarded.

| | | | | | | | | | | | | |
|---|---|---|---|---|---|---|---|---|---|---|---|---|
| Allele- | Allele- | Paired WT | | | | | | | | | | Off target |
| specific | specific | primer | Paired | | Amplicon | Tm | | GC% | self- | | | with wt |
| Primer | Primer Tm | name | WT primer sequence | Length | size | (° C.) | GC% | tail | dimers | Hairpins | Off target? | allele-specific? |

| | | | | | | | | | | | |
|---|---|---|---|---|---|---|---|---|---|---|---|
| F25L \| 19 | 56 | F25 \| P21 | CCACATAGTTCTCAAACACTG | 21 | 76 | 60 | 42.9 | 42.9 | -0.07 | 0.04 | Partial. Evaluate in silico. |
| | | F25 \| P20 | CACATAGTTCTCAAACACTG | 20 | 75 | 56 | 40.00 | 42.9 | -0.07 | 0.04 | Partial. Evaluate in silico. |
| F25L \| 18 | 54 | F25 \| P20 | CACATAGTTCTCAAACACTG | 20 | 74 | 56 | 40.00 | 42.9 | -0.07 | 0.04 | Partial. Evaluate in silico. |
| | | F25 \| P19 | ACATAGTTCTCAAACACTG | 19 | 73 | 52 | 36.80 | 42.9 | -0.07 | 0.04 | Partial. Evaluate in silico. |
| F25L \| 17 | 50 | F25 \| P20 | CACATAGTTCTCAAACACTG | 20 | 73 | 56 | 40.00 | 42.9 | -0.07 | 0.04 | Partial. Evaluate in silico. |
| | | F25 \| P19 | ACATAGTTCTCAAACACTG | 19 | 72 | 52 | 36.80 | 42.9 | -0.07 | 0.04 | Partial. Evaluate in silico. |
| F25L \| 16 | 48 | F25 \| P19 | ACATAGTTCTCAAACACTG | 19 | 71 | 52 | 36.80 | 42.9 | -0.07 | 0.04 | Partial. Evaluate in silico. |
| | | F25 \| P18 | CATAGTTCTCAAACACTG | 18 | 70 | 50 | 38.90 | 42.9 | -0.07 | 0.04 | Partial. Evaluate in silico. |

| | | | | | | | | | |
|---|---|---|---|---|---|---|---|---|---|
| Accepted values for the parameters in analysis | 16-25 | <100 | ΔTm with | 10-60% | >25% | >-4 Kcal/ | >-3 Kcal/ | no | no |
| (paired WT primer) | bp | bp | paired | | | mol | mol | | |
| | | | primer | | | | | | |
| | | | <5° C. | | | | | | |

The following combinations meet the thermodynamic criteria required:
F25L I 19+F25 I P21
F25L i 19+F25 I P20
F25L i 18+F25 I P20
F25L i 18+F25 I P19
F25L i 17+F25 I P20
F251, i 17+F25 I P19
F25L i 16+F25 I P19
F25L i 16+F25 I P18

Blat alignment of the validated primer pairs in conventional sense

>F25L_18 + F25_P20/74 bp
SEQ ID No: 137
GAACTGGTCCTTGCTGAGgactatgagcaagcatgtctttccacaggct
ccatcACCAACAATCACCAGTTTCT
>F25L_17 + F25_P20/73 bp
SEQ ID No: 138
AACTGGTCCTTGCTGAGgactatgagcaagcatgtctttccacaggctc
catcACCAACAATCACCAGTTTCT
>F25L_16 + F25_P20/72 bp

SEQ ID No: 139
ACTGGTCCTTGCTGAGgactatgagcaagcatgtctttccacaggctcc
atcACCAACAATCACCAGTTTCT

When in conventional configuration, the forward primers (F25L_18, F25L_17, F25L_16) hit a secondary region of homology on chromosome 6, with all but the allele-specific position of the primer matching the secondary target. Nevertheless, as indicated from the characteristics of the reported homology (FIG. 17), the homology region does not cover the landing site of the reverse primer, so that no amplification outside the true target region will be possible. This primer combination can hence be accepted and analysed further.

All primer pairs in conventional configuration are expected to produce just one single amplicon at the expected chromosomal position. No other homologies are reported.

The following combinations in conventional configuration meet the thermodynamic criteria required:
F25L 18 +F25L P20
F25L 17 +F25L P20
F25L 16+F25L P20

1.1.4. Blat alignment of the validated primer pairs in inverse configuration

>F25L_I_19 + F25_I_P21/76 bp
SEQ ID No: 140
CCACATAGTTCTCAAACACTGtgggcacatacacctctgggaactggt
ccttgctgaGGACTATGAGCAAGCATGT
>F25L_I_19 + F25_I_P20/75 bp
SEQ ID No: 141
CACATAGTTCTCAAACACTGtgggcacatacacctctgggaactggtc
cttgctgaGGACTATGAGCAAGCATGT
>F25L_I_18 + F25_I_P20/74 bp
SEQ ID No: 142
CACATAGTTCTCAAACACTGtgggcacatacacctctgggaactggtc
cttgctgaGGACTATGAGCAAGCATG
>F25L_I_18 + F25_I_P19/74 bp
SEQ ID No: 143
ACATAGTTCTCAAACACTGtgggcacatacacctctgggaactggtcc
ttgctgaGGACTATGAGCAAGCATG
>F25L_I_17 + F25_I_P20/73 bp
SEQ ID No: 144
CACATAGTTCTCAAACACTGtgggcacatacacctctgggaactggtc
cttgctgaGGACTATGAGCAAGCAT
>F25L_I_17 + F25_I_P19/72 bp
SEQ ID No: 145
ACATAGTTCTCAAACACTGtgggcacatacacctctgggaactggtcc
ttgctgaGGACTATGAGCAAGCAT
>F25L_I_16 + F25_I_P19/71 bp
SEQ ID No: 146
ACATAGTTCTCAAACACTGtgggcacatacacctctgggaactggtcc
ttgctgaGGACTATGAGCAAGCA
>F25L_I_16 + F25_I_P18/70 bp
SEQ ID No: 147
CATAGTTCTCAAACACTGtgggcacatacacctctgggaactggtcct
tgctgaGGACTATGAGCAAGCA

By working in inverse configuration, it is possible to observe a high homology region on chromosome 6. From closer analysis of the homology structure and distribution, it is possible to notice that there should be enough base pair differences in the primers binding site for ensuring specific amplification of amplicon of interest on chromosome 3, as highlighted in FIG. 3. Nevertheless, this aspect should be flagged during the in vitro testing, aimed at excluding any risk of secondary amplification.

Wet-lab validation will be performed preferentially on all thermodynamically suitable primer pairs in conventional configuration. In case this strategy fails the QC, all thermodynamically suitable primers in inverse configuration will be tested.

The following combinations of primers in inverse configuration can be tested as backup:
F25L I 19+F25 I P21
F25L i 19+F25 I P20
F25L i 18+F25 I P20
F25L i 18+F25 I P19
F25L i 17+F25 I P20
F25L i 17+F25 I P19
F25L i 16+F25 I P19
F25L i 16+F25 I P18

1.2. Oligoblocker Design for the Allele-Specific RHOA F25L System

1.2.1. RHOA F25 Oligoblocker Candidates in Conventional Configuration

SEQ ID No: 148 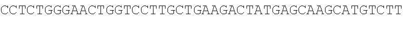
,
Wild-type target sequence for RHOA F25
oligoblocker design.

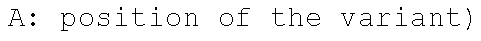

TABLE 5
List of all potential oligoblockers for the RHOA F25L codon, in conventional
configuration, considered in the design of this invention and their thermodynamic
characteristics. Note that Tm are calculated using the Oligoanalyzer ® software.
Accepted values are presented with white background. Borderline values are presented
with light grey background and non-acceptable values are presented with dark grey
background. In the eventuality that one of the parameters in analysis falls outside the
accepted range indicated in the last row the thermodynamic analysis is discontinued
and the oligoblocker is discarded

| Oligoblocker | | | Tm | | GC% | | |
|---|---|---|---|---|---|---|---|
| name | Sequence | Length | (° C.) | GC% | tail | self-dimers | Hairpins |
| F25L OB23 | TGGTCCTTGCTGAAGACTATGAG | 23 | 68 | | | | |
| F25L OB22 | GGTCCTTGCTGAAGACTATGAG | 22 | 66 | | | | |
| F25L OB21 | GTCCTTGCTGAAGACTATGAG | 21 | 62 | 47.6 | 42.9 | -0.33 | -0.21 |
| F25L OB20 | TCCTTGCTGAAGACTATGAG | 20 | 58 | 45 | 42.9 | -0.33 | -0.21 |
| F25L OB19 | CCTTGCTGAAGACTATGAG | 19 | 56 | | | | |

| | | | | | | |
|---|---|---|---|---|---|---|
| Accepted values for the parameters | 19-23 | ~60° | 40-60% | >25% | >-4 Kcal/mol | >-3 Kcal/mol |
| in analysis (Oligoblocker) | bp | C. | | | | |

RHOA F25 oligoblocker candidates in inverse configuration

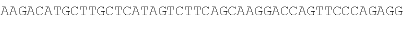

SEQ ID No: 149: Wild-type target sequence for RHOA
F25 oligoblocker design in inverse configuration.

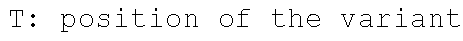

TABLE 6
List of all potential oligoblockers for the RHOA F₂₅L codon, in inverse
configuration, considered in the design of this invention and their thermodynamic
characteristics. Note that Tm are calculated using the Oligoanalyzer® software.
Accepted values are presented with white background. Borderline values are presented
with light grey background and non-acceptable values are presented with dark grey
background. In the eventuality that one of the parameters in analysis falls
outside the accepted range indicated in the last row the thermodynamic
analysis is discontinued and the oligoblocker is discarded.

| Oligoblocker | | | Tm | | GC % | self- | |
|---|---|---|---|---|---|---|---|
| name | Sequence | Length | (° C.) | GC % | tail | dimers | Hairpins |
| F25L i OB23 | TGCTCATAGTCTTCAGCAAGGAC | 23 | 68 | | | | |
| F25L i OB22 | GCTCATAGTCTTCAGCAAGGAC | 22 | 66 | | | | |
| F25L i OB21 | GCTCATAGTCTTCAGCAAGGA | 21 | 62 | 47.6 | 57.1 | −1.53 | −1.41 |
| F25L i OB21/2 | CTCATAGTCTTCAGCAAGGAC | 21 | 62 | 47.6 | 57.1 | −0.33 | −0.21 |
| F25L i OB20 | CTCATAGTCTTCAGCAAGGA | 20 | 58 | 45 | 57.1 | −0.33 | −0.21 |
| F25L i OB19 | CTCATAGTCTTCAGCAAGG | 19 | 56 | | | | |

| | | | | | | |
|---|---|---|---|---|---|---|
| Accepted values for the parameters | 19-23 bp | ~60° C. | 40-60% | >25% | > −4 | > −3 |
| in analysis (Oligoblocker) | | | | | Kcal/mol | 4Kcal/mol |

Final Thermodynamic Analysis on All Candidate Primers and Oligoblockers

Heterodimers combinations to consider for all the primers/oligoblocker candidates that met the thermodynamic criteria described in the previous sections:
Allele-specific primer+paired WT primer
Allele-specific primer+oligoblocker
Paired WT primer+oligoblocker

Thermodynamic analysis in conventional configuration

TABLE 7
Evaluation of heterodimers presence for all oligonucleotide
candidates in conventional configuration evaluated
for the allele-specific system.

| Oligonucleotide combination | ΔG | Validated? |
|---|---|---|
| (conventional) | (Kcal/mol) | Y/N |
| F25L 18 + F25 P20 | −0.07 | Y |
| F25L 18 + F25L OB 21 | −0.33 | Y |
| F25L 18 + F25L OB 20 | −0.33 | Y |
| F25L 17 + F25 P20 | −0.07 | Y |
| F25L 17 + F25L OB 21 | −0.33 | Y |
| F25L 17 + F25L OB 20 | −0.33 | Y |
| F25L 16 + F25 P20 | none | Y |
| F25L 16 + F25L OB 21 | −0.33 | Y |
| F25L 16 + F25L OB 20 | −0.33 | Y |
| F25 P20 + F25L OB 21 | none | Y |
| F25 P20 + F25L OB 20 | none | Y |
| Accepted values for the | >−4 Kcal/mol | Y |
| parameters in analysis | | |

All oligonucleotide candidates in conventional configuration meet the thermodynamic criteria required to ensure absence of non-specific amplification and to improve the amplification efficiency.

Thermodynamic analysis in inverse configuration

TABLE 8
Evaluation of heterodimers presence for all oligonucleotide candidates
in conventional configuration evaluated for the allele-specific
system. Accepted values are presented with white background and
non-acceptable values are presented with dark grey background.

| Oligonucleotide combination | ΔG | Validated? |
|---|---|---|
| (inverse) | (Kcal/mol) | Y/N |
| F25L I 19 + F25 I P21 | −0.69 | Y |
| F25L I 19 + F25 I P20 | −0.69 | Y |
| F25L I 18 + F25 I P20 | −0.69 | Y |
| F25L I 18 + F25 I P19 | −0.69 | Y |
| F25L I 17 + F25 I P20 | −0.69 | Y |
| F25L I 17 + F25 I P19 | −0.69 | Y |
| F25L I 16 + F25 I P19 | −0.69 | Y |
| F25L I 16 + F25 I P18 | −0.69 | Y |
| F25L I 19 + F25L i OB21 | −7.03 | N |
| F25L I 19 + F25L i OB21/2 | −7.03 | N |
| F25L I 19 + F25L i OB20 | −7.03 | N |
| F25L I 18 + F25L i OB21 | −7.03 | N |
| F25L I 18 + F25L i OB21/2 | −7.03 | N |
| F25L I 18 + F25L i OB20 | −7.03 | N |
| F25L I 17 + F25L i OB21 | −7.03 | N |
| F25L I 17 + F25L i OB21/2 | −7.03 | N |
| F25L I 17 + F25L i OB20 | −7.03 | N |
| F25L I 16 + F25L i OB21 | −7.03 | N |
| F25L I 16 + F25L i OB21/2 | −7.03 | N |
| F25L I 16 + F25L i OB20 | −7.03 | N |
| F25 I P21 + F25L i OB21 | −0.34 | Y |
| F25 I P21 + F25L i OB21/2 | −0.34 | Y |
| F25 I P21 + F25L i OB20 | −0.34 | Y |
| F25 I P20 + F25L i OB21 | −0.34 | Y |
| F25 I P20 + F25L i OB21/2 | −0.34 | Y |
| F25 I P20 + F25L i OB20 | −0.34 | Y |
| F25 I P19 + F25L i OB21 | −0.34 | Y |
| F25 I P19 + F25L i OB21/2 | −0.34 | Y |
| F25 I P19 + F25L i OB20 | −0.34 | Y |
| F25 I P18 + F25L i OB21 | −0.34 | Y |
| F25 I P18 + F25L i OB21/2 | −0.34 | Y |
| F25 I P18 + F25L i OB20 | −0.34 | Y |
| Accepted values for the | >−4 Kcal/mol | Y |
| parameters in analysis | | |

All oligoblockers designed in inverse configuration do not meet the thermodynamic criteria required to ensure absence of primer dimers.

New oligoblockers should be designed in inverse configuration.

New Oligoblocker Design in Inverse Configuration

Given the results obtained in the previous paragraph, a new set of oligoblockers was generated in silico shifting the position of the wild-type allele towards the oligoblocker 3′ to avoid the region of homology with the allele specific primers.

TABLE 9
List of all potential oligoblockers for the RHOA F25L codon, in inverse
configuration, considered in the design of this invention and their thermodynamic

| Oligoblocker | | | Tm | | GC% | self- | |
|---|---|---|---|---|---|---|---|
| name | Sequence | Length | (° C.) | GC% | tail | dimers | Hairpins |
| F25L i OB23/2 | ACATGCTTGCTCATAGTCTTCAG | 23 | 66 | | | | |
| F25L i OB22/2 | CATGCTTGCTCATAGTCTTCAG | 22 | 64 | | | | |
| F25L i OB21/3 | ATGCTTGCTCATAGTCTTCAG | 21 | 60 | 42.9 | 42.9 | -0.22 | -0.1 |
| F25L i OB20/2 | TGCTTGCTCATAGTCTTCAG | 20 | 58 | 45 | 42.9 | none | none |
| F25L i OB19/2 | GCTTGCTCATAGTCTTCAG | 19 | 56 | | | | |

| | | | | | | |
|---|---|---|---|---|---|---|
| Accepted values for the parameters | 19-23 | ~60° | 40-60% | >25% | >-4 Kcal/ | >-3 Kcal/ |
| in analysis (Oligoblocker) | bp | C. | | | mol | mol |

characteristics. Note that Tm are calculated using the Oligoanalyzer® software. Accepted values are presented with white background. Borderline values are presented with light grey background and non-acceptable values are presented with dark grey background. In the eventuality that one of the parameters in analysis falls outside the accepted range indicated in the last row the thermodynamic analysis is discontinued and the oligoblocker is discarded.

New Thermodynamic Analysis in Inverse Configuration

TABLE 10
Evaluation of heterodimers presence for all oligonucleotide candidates
in conventional configuration evaluated for the allele-specific
system. Accepted values are presented with white background.

| Oligonucleotide combination | ΔG | Validated? |
|---|---|---|
| (inverse) | (Kcal/mol) | Y/N |
| F25L I 19 + F25L i OB21/3 | −0.22 | Y |
| F25L I 19 + F25L i OB20/2 | −0.22 | Y |
| F25L I 18 + F25L i OB21/3 | −0.22 | Y |
| F25L I 18 + F25L i OB20/2 | −0.22 | Y |
| F25L I 17 + F25L i OB21/3 | −0.22 | Y |
| F25L I 17 + F25L i OB20/2 | −0.22 | Y |
| F25L I 16 + F25L i OB21/3 | −0.22 | Y |
| F25L I 16 + F25L i OB20/2 | none | Y |
| F25 I P21 + F25L i OB21/3 | −0.69 | Y |
| F25 I P21 + F25L i OB20/2 | −0.69 | Y |
| F25 I P20 + F25L i OB21/3 | −0.69 | Y |
| F25 I P20 + F25L i OB20/2 | −0.69 | Y |
| F25 I P19 + F25L i OB21/3 | −0.69 | Y |
| F25 I P19 + F25L i OB20/2 | −0.69 | Y |
| F25 I P18 + F25L i OB21/3 | −0.69 | Y |
| F25 I P18 + F25L i OB20/2 | −0.69 | Y |
| Accepted values for the | >−4 Kcal/mol | Y |
| parameters in analysis | | |

Design of the F25L Wild-Type Set

RHOA F25 Wild-Type Set Candidates

TABLE 11
Evaluation of all thermodynamic parameters considered to select appropriate
oligonucleotides for the RHOA F25 wild-type set. Accepted values are presented with
white background.

| Primer | | | Tm | | GC% | self- | | | Validated |
|---|---|---|---|---|---|---|---|---|---|
| name | Sequence | Length | (° C.) | GC% | tail | dimers | Hairpins | Heterodimers | (Y/N) |
| F25 WT L | GCAGGATGAGAATGGATTC | 19 | 56 | 47.4 | 42.9 | -1.78 | -0.19 (32.3° C.) | -1.88 | Y |
| F25 WT R | GAATTAGAGCTTTTTGCCTC | 20 | 56 | 40 | 57.1 | -3.13 | 0.16 (42.8° C.) | | Y |

| | | | | | | | | |
|---|---|---|---|---|---|---|---|---|
| Accepted values for the parameters | 16-22 | 55-60°C | 40-60% | >25% | >-4 Kcal/ | >-3 Kcal/ | >-4 Kcal/ | Y |
| in analysis (wild-type primer set) | bp | ΔTm with | | | mol | mol | mol | |
| | | paired | | | | | | |

Blat Alignment of the Validated Primer Pairs in Conventional Sense

The Blat local alignment (as shown in FIG. 4) highlighted the presence of secondary homology regions that are not spanning the primers landing sites. No secondary product is expected to be produced. This primer combination can hence be validated.

The oligonucleotide candidates of the RHOA 25 WT set meet the thermodynamic criteria required to ensure absence of non-specific amplification and to improve the amplification efficiency.

RHOA G17V

The inventors have designed specific sets of primers allowing improved detection of the presence (or absence, or frequency) of such a mutation from a sample containing cfNA or genomic DNA.

More specifically, in a preferred embodiment, the mutation is RHOA G17V, where the glucine to valine change is due to a single point mutation at position chr3: 49412973 (human assembly GRCh37/hg19) where the wild-type allele is a cytosine (C) and the mutant allele is an adenine (A). The first set of reagents for detecting the RHOA G17V mutation comprises a mutation-specific primer selected from SEQ ID NO: 39-52, a paired primer selected from SEQ ID NO: 53-59, and an oligoblocker selected from SEQ ID NO: 60-69. Most preferably, the mutation-specific primer is selected from SEQ ID NO: 43-44, 50-51, the paired primer is selected from SEQ ID NO: 53-54, 58-59 and the oligoblocker is selected from SEQ ID NO: 62-64, 67-69. In another embodiment, a different paired primer can be chosen from ANY consecutive DNA sequence of size comprised between 15 and 25 base pairs selected in the following regions: chr3: 49412738-49412948 and/or chr3: 49412998-49413208 (human assembly GRCh37/hg19). A different oligoblocker can be chosen from ANY consecutive DNA sequence of size comprised between 15 and 25 base pairs selected in the following regions: chr3: 49412948-49412998 (human assembly GRCh37/hg19) with the fundamental prerequisite that the position chr3: 49412973 must be included in the selected oligoblocker sequence. In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 70 and a second primer SEQ ID NO: 71.

Primer Design for the Allele-Specific RHOA G17V System

The allele-specific primers considered in the design of this invention and their thermodynamic characteristics are listed in Table 12:

TABLE 12
List of all the RHOA G17V allele-specific primers considered in the design of
this invention and their thermodynamic characteristics. Accepted values are presented
with white background. Borderline values are presented with light grey background and
non-acceptable values are presented with dark grey background. In the eventuality that
one of the parameters in analysis falls outside the accepted range indicated in the last
row the thermodynamic analysis is discontinued and the primer is discarded.

| | | | | | | | |
|---|---|---|---|---|---|---|---|
| Primer name | Sequence | Length | Tm (° C.) | GC% | GC% tail | self-dimers | Hairpins |

Conventional configuration

| | | | | | | | |
|---|---|---|---|---|---|---|---|
| G17V 22 | ACTATGAGCAAGCATGTCTTTA | 22 | 60 | | | | |
| G17V 21 | CTATGAGCAAGCATGTCTTTA | 21 | 58 | | | | |
| G17V 20 | TATGAGCAAGCATGTCTTTA | 20 | 54 | 35 | 28.6 | -2.17 | -0.21 |
| G17V 19 | ATGAGCAAGCATGTCTTTA | 19 | 52 | 36.8 | 28.6 | -2.17 | -0.21 |
| G17V 18 | TGAGCAAGCATGTCTTTA | 18 | 50 | 38.9 | 28.6 | -2.17 | -0.21 |
| G17V 17 | GAGCAAGCATGTCTTTA | 17 | 48 | 41.2 | 28.6 | -2.17 | -0.21 |
| G17V 16 | AGCAAGCATGTCTTTA | 16 | 44 | 37.5 | 28.6 | -2.17 | -0.21 |

Inverse configuration

| | | | | | | | |
|---|---|---|---|---|---|---|---|
| G17V \| 22 | TTGTTGGTGATGGAGCCTGTGT | 22 | 66 | | | | |
| G17V \| 21 | TGTTGGTGATGGAGCCTGTGT | 21 | 64 | | | | |
| G17V \| 20 | GTTGGTGATGGAGCCTGTGT | 20 | 62 | | | | |
| G17V \| 19 | TTGGTGATGGAGCCTGTGT | 19 | 58 | | | | |
| G17V \| 18 | TGGTGATGGAGCCTGTGT | 18 | 56 | 55.6 | 57.1 | none | none |
| G17V \| 17 | GGTGATGGAGCCTGTGT | 17 | 54 | 58.8 | 58.1 | none | none |
| G17V \| 16 | GTGATGGAGCCTGTGT | 16 | 50 | 56.3 | 59.1 | none | none |

| | | | | | | |
|---|---|---|---|---|---|---|
| Accepted values for the parameters | 16-22 | 45-55° | 40-60% | >25% | >-4 Kcal/ | >-3 Kcal/ |
| in analysis (allele-specific orimer) | bp | C. | | | mol | mol |

RHOA G17V

The inventors have designed specific sets of primers allowing improved detection of the presence (or absence, or frequency) of such a mutation from a sample containing cfNA or genomic DNA.

More specifically, in a preferred embodiment, the mutation is RHOA G17V, where the glycine to valine change is due to a single point mutation at position chr3: 49412973 (human assembly GRCh37/hg19) where the wild-type allele is a cytosine (C) and the mutant allele is an adenine (A). The first set of reagents for detecting the RHOA G17V mutation comprises a mutation-specific primer selected from SEQ ID NO: 39-52, a paired primer selected from SEQ ID NO: 53-59, and an oligoblocker selected from SEQ ID NO: 60-69. Most preferably, the mutation-specific primer is selected from SEQ ID NO: 43-44, 50-51, the paired primer is selected from SEQ ID NO: 53-54, 58-59 and the oligoblocker is selected from SEQ ID NO: 62-64, 67-69. In another embodiment, a different paired primer can be chosen from ANY consecutive DNA sequence of size comprised between 15 and 25 base pairs selected in the following regions: chr3: 49412738-49412948 and/or chr3: 49412998-49413208 (human assembly GRCh37/hg19). A different oligoblocker can be chosen from ANY consecutive DNA sequence of size comprised between 15 and 25 base pairs selected in the following regions: chr3: 49412948-49412998 (human assembly GRCh37/hg19) with the fundamental prerequisite that the position chr3: 49412973 must be included in the selected oligoblocker sequence. In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 70 and a second primer SEQ ID NO: 71.

Primer Design for the Allele-Specific RHOA G17V System

The allele-specific primers considered in the design of this invention and their thermodynamic characteristics are listed in Table 13:

TABLE 13
List of all the RHOA G17V allele-specific primers considered in the design of
this invention and their thermodynamic characteristics. Accepted values are presented
with white background. Borderline values are presented with light grey background and
non-acceptable values are presented with dark grey background. In the eventuality that
one of the parameters in analysis falls outside the accepted range indicated in the last
row the thermodynamic analysis is discontinued and the primer is discarded.

| | | | | | | | |
|---|---|---|---|---|---|---|---|
| Primer name | Sequence | Length | Tm (° C.) | GC% | GC% tail | self-dimers | Hairpins |

Conventional configuration

| | | | | | | | |
|---|---|---|---|---|---|---|---|
| G17V 22 | ACTATGAGCAAGCATGTCTTTA | 22 | 60 | | | | |
| G17V 21 | CTATGAGCAAGCATGTCTTTA | 21 | 58 | | | | |
| G17V 20 | TATGAGCAAGCATGTCTTTA | 20 | 54 | 35 | 28.6 | -2.17 | -0.21 |
| G17V 19 | ATGAGCAAGCATGTCTTTA | 19 | 52 | 36.8 | 28.6 | -2.17 | -0.21 |
| G17V 18 | TGAGCAAGCATGTCTTTA | 18 | 50 | 38.9 | 28.6 | -2.17 | -0.21 |
| G17V 17 | GAGCAAGCATGTCTTTA | 17 | 48 | 41.2 | 28.6 | -2.17 | -0.21 |
| G17V 16 | AGCAAGCATGTCTTTA | 16 | 44 | 37.5 | 28.6 | -2.17 | -0.21 |

Inverse configuration

| | | | | | | | |
|---|---|---|---|---|---|---|---|
| G17V \| 22 | TTGTTGGTGATGGAGCCTGTGT | 22 | 66 | | | | |
| G17V \| 21 | TGTTGGTGATGGAGCCTGTGT | 21 | 64 | | | | |
| G17V \| 20 | GTTGGTGATGGAGCCTGTGT | 20 | 62 | | | | |
| G17V \| 19 | TTGGTGATGGAGCCTGTGT | 19 | 58 | | | | |
| G17V \| 18 | TGGTGATGGAGCCTGTGT | 18 | 56 | 55.6 | 57.1 | none | none |
| G17V \| 17 | GGTGATGGAGCCTGTGT | 17 | 54 | 58.8 | 58.1 | none | none |
| G17V \| 16 | GTGATGGAGCCTGTGT | 16 | 50 | 56.3 | 59.1 | none | none |

| | | | | | | |
|---|---|---|---|---|---|---|
| Accepted values for the parameters | 16-22 | 45-55° | 40-60% | >25% | >-4 Kcal/ | >-3 Kcal/ |
| in analysis (allele-specific orimer) | bp | C. | | | mol | mol |

Paired Wild-Type Primer

Results Obtained with Primer 3

All allele-specific primers that met the IntPlex® criteria (see Table 13) were used on Primer3 to find a compatible paired primer. Results are summarised in Table 14.

TABLE 14
List of all the RHOA G17V paired WT primers produced by Primer 3 and
considered in the design of this invention and their thermodynamic characteristics.
Note that Tm are calculated following the recommended Primers algorithm. Accepted
values are presented with white background and borderline values are presented with
light grey background. In the eventuality that one of the parameters in analysis falls

| Allele- | Allele-specific | Paired WT | | | | | | | | | | Off target |
|---|---|---|---|---|---|---|---|---|---|---|---|---|
| specific | PrimerTm | primer | Paired | | Amplicon | Tm | | GC% | self- | | Off | with wt allele- |
| Primer | (Primer3) | name | WT primer sequence | Length | size | (° C.) | GC% | tail | dimers | Hairpins | target? | specific? |
| G17V \| 18 | 58.09 | G17 \| P18 | TTCTCAAACACTGTGGGC | 18 | 90 | 55.06 | 50 | 71.4 | -0.08 | none | no | no |
| | | G17 \| P19 | GAACTGGTCCTTGCTGAAG | 19 | 58 | 55.84 | 52.63 | 57.1 | -0.33 | -0.21 | no | no |
| G17V \| 17 | 55.95 | G17 \| P18 | TTCTCAAACACTGTGGGC | 18 | 89 | 55.06 | 50 | 71.4 | -0.08 | none | no | no |
| | | G17 \| P19 | GAACTGGTCCTTGCTGAAG | 19 | 57 | 55.84 | 52.63 | 57.1 | -0.33 | -0.21 | no | no |
| G17V \| 16 | 52.65 | G17 \| P20 | TCTGCCACATAGTTCTCAAA | 20 | 100 | 54.29 | 40 | 28.6 | none | none | no | no |

| | | | | | | | | | |
|---|---|---|---|---|---|---|---|---|---|
| Accepted values forthe parameters | 16-25 | <100 bp | ΔTm with | 40-60% | >25% | >-4 Kcal/ | -3 Kcal/ | no | no |
| in analysis (paired WT primer) | bp | | paired | | | mol | mol | | |
| | | | primer | | | | | | |
| | | | <5’C | | | | | | |

outside the accepted range indicated in the last row the thermodynamic analysis is discontinued and the primer is discarded.

From the results presented in Table 14 it was possible to identify potential candidates as paired WT primer for the RHOA G17V allele-specific primers G17V I 18, G17V I 17 and G17V I 16 in inverse configuration.

On the other hand, Primer3 did not retrieve any candidate in conventional configuration.

The paired-wild type primer in conventional configuration may be manually designed following the thermodynamic criteria described above.

The paired WT primers G17 I P20, G17 I P19 and G17 I P18 in inverse configuration is suitable for in vitro evaluation, in combination with the allele-specific primers: G17V I 18, G17V I 17 and G17V I 16.

Manually Generated Primer Pair Candidates in Conventional Configuration

TABLE 15
List of potential RHOA G17 paired WT- primers in conventional configuration
manually generated and considered in the design of this invention and their
thermodynamic characteristics. Note that Tm are calculated using the Oligoanalyzer®
software. Accepted values are presented with white background and borderline values
are presented with light grey background. In the eventuality that one of the parameters
in analysis falls outside the accepted range indicated in the last row the thermodynamic
analysis is discontinued and the primer is discarded.

| Allele- | Allele- | | | | | | | | | | | Off target |
|---|---|---|---|---|---|---|---|---|---|---|---|---|
| specific | specific | Paired WT | Paired | | Amplicon | Tm | | GC% | self- | | Off | with wt allele- |
| Primer | Primer Tm | primer name | WT primer sequence | Length | size | (° C.) | GC% | tail | dimers | Hairpins | target? | specific? |
| G17V 20 | 54 | G17 P21 | TCTGTGTTTTGTGTTTCAGCA | 21 | 90 | 58 | 38.1 | 42.9 | -0.34 | -0.22 | no | no |
| | | G17 P20 | CTGTGTTTTGTGTTTCAGCA | 20 | 89 | 56 | 40.00 | 42.9 | -0.34 | -0.22 | no | no |
| G17V 19 | 52 | G17 P21 | TCTGTGTTTTGTGTTTCAGCA | 21 | 89 | 58 | 38.1 | 42.9 | -0.34 | -0.22 | no | no |
| | | G17 P20 | CTGTGTTTTGTGTTTCAGCA | 20 | 88 | 56 | 40.00 | 42.9 | -0.34 | -0.22 | no | no |
| G17V 18 | 50 | G17 P20 | CTGTGTTTTGTGTTTCAGCA | 20 | 87 | 56 | 40.00 | 42.9 | -0.34 | -0.22 | no | no |
| | | G17 P19 | TGTGTTTTGTGTTTCAGCA | 19 | 86 | 52 | 36.80 | 42.9 | none | none | no | no |
| G17V 17 | 48 | G17 P19 | TGTGTTTTGTGTTTCAGCA | 19 | 85 | 52 | 36.80 | 42.9 | none | none | no | no |
| | | G17 P18 | GTGTTTTGTGTTTCAGCA | 18 | 84 | 50 | 38.90 | 42.9 | none | none | no | no |

| | | | | | | | | | |
|---|---|---|---|---|---|---|---|---|---|
| Accepted values for the parameters | 16-25 | <100 bp | ΔTm with | 10-60% | >25% | >-4 Kcal/ | >-3 Kcal/ | no | no |
| in analysis (paired WT primer) | bp | | paired | | | mol | mol | | |
| | | | primer | | | | | | |
| | | | <5° C. | | | | | | |

The following combinations meet the thermodynamic criteria required:
G17V 20+G17 P21
G17V 20+G17 P20
G17V 19+G17 P21
G17V 19+G17 P20
G17V 18+G17 P20
G17V 18+G17 P19
G17V 17+G17 P19
G17V 17+G17 P18

Blat alignment of the validated primer pairs in conventional sense

>G17V_20 + G17_P21/90 bp
SEQ ID No: 150
TATGAGCAAGCATGTCTTTAcacaggctccatcaccaacaatcaccagt
ttcttccggatggcagccatTGCTGAAACACAAAACACAGA
>G17V_20 + G17_P20/89 bp
SEQ ID No: 151
TATGAGCAAGCATGTCTTTAcacaggctccatcaccaacaatcaccagt
ttcttccggatggcagccatTGCTGAAACACAAAACACAG
>G17V_19 + G17_P21/89 bp
SEQ ID No: 152
ATGAGCAAGCATGTCTTTAcacaggctccatcaccaacaatcaccagtt
tcttccggatggcagccatTGCTGAAACACAAAACACAGA
>G17V_19 + G17_P20/88 bp
SEQ ID No: 153
ATGAGCAAGCATGTCTTTAcacaggctccatcaccaacaatcaccagtt
tcttccggatggcagccatTGCTGAAACACAAAACACAG
>G17V_18 + G17_P20/87 bp
SEQ ID No: 154
TGAGCAAGCATGTCTTTAcacaggctccatcaccaacaatcaccagttt
cttccggatggcagccatTGCTGAAACACAAAACACAG
>G17V_18 + G17_P19/86 bp
SEQ ID No: 155
TGAGCAAGCATGTCTTTAcacaggctccatcaccaacaatcaccagttt
cttccggatggcagccatTGCTGAAACACAAAACACA
>G17V_17 + G17_P19/85 bp
SEQ ID No: 156
GAGCAAGCATGTCTTTAcacaggctccatcaccaacaatcaccagtttc
ttccggatggcagccatTGCTGAAACACAAAACACA
>G17V_17 + G17_P18/84 bp
SEQ ID No: 157
GAGCAAGCATGTCTTTAcacaggctccatcaccaacaatcaccagtttc
ttccggatggcagccatTGCTGAAACACAAAACAC

When in conventional configuration, all the paired primers hit two secondary regions of homology on chromosome 6, with all but the allele-specific position of the primer matching the secondary target. Nevertheless, as indicated from the characteristics of the reported homology (FIG. 5), the homology region does not cover the landing site of the forward primer, so that no amplification outside the true target region will be possible. These primer combinations can hence be accepted and analysed further (see FIG. 6.

All primer pairs in conventional configuration are expected to produce just one single amplicon at the expected chromosomal position. No other homologies are reported.

All tested combinations in conventional configuration meet the thermodynamic criteria required.

Blat alignment of the validated primer pairs in inverse configuration (FIG. 7)

>G17V_I18 + G17_IP18/90 bp
SEQ ID No: 158
TTCTCAAACACTGTGGGCacatacacctctgggaactggtccttgctga
agactatgagcaagcatgtctttACACAGGCTCCATCACCA
>G17V_I18 + G17_IP19/58 bp
SEQ ID No: 159
GAACTGGTCCTTGCTGAAGactatgagcaagcatgtctttACACAGGCT
CCATCACCA
>G17V_I17 + G17_IP18/89 bp
SEQ ID No: 160
TTCTCAAACACTGTGGGCacatacacctctgggaactggtccttgctga
agactatgagcaagcatgtctttACACAGGCTCCATCACC
>G17V_I17 + G17_IP19/57 bp
SEQ ID No: 161
GAACTGGTCCTTGCTGAAGactatgagcaagcatgtctttACACAGGCT
CCATCACC
>G17V_I16 + G17_IP20/100 bp
SEQ ID No: 162
TCTGCCACATAGTTCTCAAAcactgtgggcacatacacctctgggaact
ggtccttgctgaagactatgagcaagcatgtctttACACAGGCTCCATC
AC

G17V_I16+G17_IP20/100 bp: This combination is OK, as the homology does not affect any of the primer binding sites.

G17V_I17+G17_IP18/89 bp: This combination is OK, as the homology does not affect any of the primer binding sites.

G17V_I17+G17_IP19/57 bp: homology region on chromosome 6 that affects the paired primer binding site. Nevertheless, the allele-specific primer is specific so no non-specific product should be amplified.

G17V_I18+G17_IP18/90 bp: This combination is OK, as the homology does not affect any of the primer binding sites.

G17V_I18+G17_IP19/58 bp: homology region on chromosome 6 that affects the paired primer binding site. Nevertheless, the allele-specific primer is specific so no non-specific product should be amplified.

By working in inverse configuration, it is possible to observe a homology region on chromosome 6 when using the paired wt primer G17 I P19 (FIG. 8). It is important to notice that the allele-specific primer is not included in the homology region so this is unlikely to generate non-specific products. Nevertheless, this aspect should be flagged during the in vitro testing, aimed at excluding any risk of secondary amplification.

Wet-lab validation will be performed preferentially on all thermodynamically suitable primer pairs

Oligoblocker Design for the Allele-Specific RHOA G17V System

RHOA G17 Oligoblocker Candidates in Conventional Configuration

SEQ ID No: 163

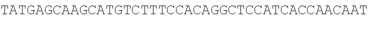

Wild-type target sequence for RHOA F25
oligoblocker design.

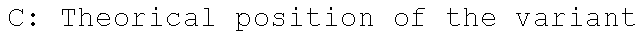

TABLE 16
List of all potential oligoblockers for the RHOA G₁₇ codon, in conventional
configuration, considered in the design of this invention and their thermodynamic
characteristics. Note that Tm are calculated using the Oligoanalyzer® software.
Accepted values are presented with white background. Borderline values are presented
with light grey background and non-acceptable values are presented with dark grey
background. In the eventuality that one of the parameters in analysis falls
outside the accepted range indicated in the last row the thermodynamic analysis
is discontinued and the oligoblocker is discarded

| Oligoblocker | | | Tm | | GC % | self- | |
|---|---|---|---|---|---|---|---|
| name | Sequence | Length | (° C.) | GC % | tail | dimers | Hairpins |
| G17 OB23 | CATGTCTTTCCACAGGCTCCATC | 23 | 70 | | | | |
| G17 OB22 | ATGTCTTTCCACAGGCTCCATC | 22 | 66 | | | | |
| G17 OB21 | ATGTCTTTCCACAGGCTCCAT | 21 | 62 | 47.6 | 57.1 | −0.22 | −0.1 |
| G17 OB20 | ATGTCTTTCCACAGGCTCCA | 20 | 60 | 50 | 71.4 | −0.08 | 0.04 |
| G17 OB19 | ATGTCTTTCCACAGGCTCC | 19 | 58 | 52.6 | 71.4 | −0.08 | 0.04 |

| | | | | | | |
|---|---|---|---|---|---|---|
| Accepted values for the parameters | 19-23 bp | ~60° C. | 40-60% | >25% | > −4 | > −3 |
| in analysis (Oligoblocker) | | | | | Kcal/mol | Kcal/mol |

RHOA G17 Oligoblocker Candidates in Inverse Configuration

SEQ ID No: 164

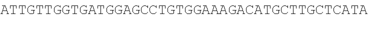

Wild-type target sequence for RHOA G17
oligoblocker design in inverse configuration.

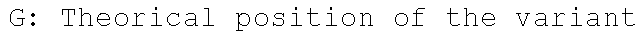

TABLE 17
List of all potential oligoblockers for the RHOA G₁₇ codon, in inverse
configuration, considered in the design of this invention and their thermodynamic
characteristics. Note that Tm are calculated using the Oligoanalyzer® software.
Accepted values are presented with white background. Borderline values are presented
with light grey background and non-acceptable values are presented with dark grey
background. In the eventuality that one of the parameters in analysis falls
outside the accepted range indicated in the last row the thermodynamic analysis
is discontinued and the oligoblocker is discarded.

| Oligoblocker | | | Tm | | GC % | self- | |
|---|---|---|---|---|---|---|---|
| name | Sequence | Length | (° C.) | GC % | tail | dimers | Hairpins |
| G17 \| OB23 | ATGGAGCCTGTGGAAAGACATGC | 23 | 70 | | | | |
| G17 \| OB22 | ATGGAGCCTGTGGAAAGACATG | 22 | 66 | | | | |
| G17 \| OB21 | ATGGAGCCTGTGGAAAGACAT | 21 | 62 | 47.6 | 28.6 | −0.22 | −0.1 |
| G17 \| OB20 | ATGGAGCCTGTGGAAAGACA | 20 | 60 | 50 | 28.6 | −0.08 | 0.04 |
| G17 \| OB19 | TGGAGCCTGTGGAAAGACA | 19 | 58 | 52.6 | 28.6 | −0.08 | 0.04 |

| | | | | | | |
|---|---|---|---|---|---|---|
| Accepted values for the parameters | 19-23 bp | ~60° C. | 40-60% | >25% | > −4 | > −3 |
| in analysis (Oligoblocker) | | | | | Kcal/mol | Kcal/mol |

Final Thermodynamic Analysis on All Candidate Primers and Oligoblockers

Heterodimers combinations to consider for all the primers/oligoblocker candidates that met the thermodynamic criteria described in the previous sections:
Allele-specific primer+paired WT primer
Allele-specific primer+oligoblocker
Paired WT primer+oligoblocker

Thermodynamic analysis in conventional configuration

TABLE 18
Evaluation of heterodimers presence for all oligonucleotide
candidates in conventional configuration evaluated
for the allele-specific system.

| Oligonucleotide combination | ΔG | Validated? |
|---|---|---|
| (conventional) | (Kcal/mol) | Y/N |
| G17V 20 + G17 P21 | −0.69 | Y |
| G17V 20 + G17 P20 | −0.69 | Y |
| G17V 20 + G17 OB21 | −3.1 | Y |
| G17V 20 + G17 OB20 | −3.1 | Y |
| G17V 20 + G17 OB19 | −3.1 | Y |
| G17V 19 + G17 P21 | −0.69 | Y |
| G17V 19 + G17 P20 | −0.69 | Y |
| G17V 19 + G17 OB21 | −3.1 | Y |
| G17V 19 + G17 OB20 | −3.1 | Y |
| G17V 19 + G17 OB19 | −3.1 | Y |
| G17V 18 + G17 P20 | −0.69 | Y |
| G17V 18 + G17 P19 | −0.69 | Y |
| G17V 18 + G17 OB21 | −3.1 | Y |
| G17V 18 + G17 OB20 | −3.1 | Y |
| G17V 18 + G17 OB19 | −3.1 | Y |
| G17V 17 + G17 P19 | −0.69 | Y |
| G17V 17 + G17 P18 | −0.69 | Y |
| G17V 17 + G17 OB21 | −3.1 | Y |
| G17V 17 + G17 OB20 | −3.1 | Y |
| G17V 17 + G17 OB19 | −3.1 | Y |
| G17 P21 + G17 OB21 | −3.64 | N |
| G17 P21 + G17 OB20 | −3.64 | N |
| G17 P21 + G17 OB19 | −3.64 | N |
| G17 P20 + G17 OB21 | −3.64 | N |
| G17 P20 + G17 OB20 | −3.64 | N |
| G17 P20 + G17 OB19 | −3.64 | N |
| G17 P19 + G17 OB21 | −2.04 | Y |
| G17 P19 + G17 OB20 | −2.04 | Y |
| G17 P19 + G17 OB19 | −2.04 | Y |
| G17 P18 + G17 OB21 | −2.04 | Y |
| G17 P18 + G17 OB20 | −2.04 | Y |
| G17 P18 + G17 OB19 | −2.04 | Y |
| Accepted values for the | >−4 Kcal/mol | Y |
| parameters in analysis | | |

The oligonucleotide combinations that passed the thermodynamic QC analysis are:

G17V 18 + G17 P19 + G17 OB21
G17V 18 + G17 P19 + G17 OB20
G17V 18 + G17 P19 + G17 OB19
G17V 17 + G17 P19 + G17 OB21
G17V 17 + G17 P19 + G17 OB20
G17V 17 + G17 P19 + G17 OB19
G17V 18 + G17 P18 + G17 OB21
G17V 18 + G17 P18 + G17 OB20
G17V 18 + G17 P18 + G17 OB19
G17V 17 + G17 P18 + G17 OB21
G17V 17 + G17 P18 + G17 OB20
G17V 17 + G17 P18 + G17 OB19

All the oligonucleotide candidates in conventional configuration of the table above meet the thermodynamic criteria required to ensure absence of non-specific amplification to and to improve the amplification efficiency.

It should be reported and considered in the following in vitro validation phases that all possible allele-specific primers form borderline hetero-dimers with the oligoblockers in conventional configuration. This cannot be avoided given the mutation position.

Thermodynamic analysis in inverse configuration

TABLE 19
Evaluation of heterodimers presence for all oligonucleotide candidates
in conventional configuration evaluated for the allele-specific
system. Accepted values are presented with white background and
non-acceptable values are presented with dark grey background.

| Oligonucleotide combination | ΔG | Validated? |
|---|---|---|
| (inverse) | (Kcal/mol) | Y/N |
| G17V I 18 + G17 I P18 | −3 | Borderline |
| G17V I 18 + G17 I P19 | −1.43 | Y |
| G17V I 18 + G17 I OB21 | −0.22 | Y |
| G17V I 18 + G17 I OB20 | −0.08 | Y |
| G17V I 18 + G17 I OB19 | −0.08 | Y |
| G17V I 17 + G17 I P18 | −3 | Borderline |
| G17V I 17 + G17 I P19 | −1.53 | Y |
| G17V I 17 + G17 I OB21 | −0.22 | Y |
| G17V I 17 + G17 I OB20 | −0.08 | Y |
| G17V I 17 + G17 I OB19 | −0.08 | Y |
| G17V I 16 + G17 I P20 | −2.04 | Y |
| G17V I 16 + G17 I OB21 | −0.22 | Y |
| G17V I 16 + G17 I OB20 | −0.08 | Y |
| G17V I 16 + G17 I OB19 | −0.08 | Y |
| G17 I P18 + G17 I OB21 | −3 | Borderline |
| G17 I P18 + G17 I OB20 | −3 | Borderline |
| G17 I P18 + G17 I OB19 | −3 | Borderline |
| G17 I P19 + G17 I OB21 | −1.53 | Y |
| G17 I P19 + G17 I OB20 | −1.53 | Y |
| G17 I P19 + G17 I OB19 | −1.53 | Y |
| G17 I P20 + G17 I OB21 | −5.11 | N |
| G17 I P20 + G17 I OB20 | −5.11 | N |
| G17 I P20 + G17 I OB19 | −5.11 | N |
| Accepted values for the | >−4 Kcal/mol | Y |
| parameters in analysis | | |

The oligonucleotide combinations that passed the thermodynamic QC analysis are:

G17V I 18 + G17 I P18 + G17 I OB21
G17V I 18 + G17 I P18 + G17 I OB20
G17V I 18 + G17 I P18 + G17 I OB19
G17V I 18 + G17 I P19 + G17 I OB21
G17V I 18 + G17 I P19 + G17 I OB20
G17V I 18 + G17 I P19 + G17 I OB19
G17V I 17 + G17 I P18 + G17 I OB21
G17V I 17 + G17 I P18 + G17 I OB20
G17V I 17 + G17 I P18 + G17 I OB19
G17V I 17 + G17 I P19 + G17 I OB21
G17V I 17 + G17 I P19 + G17 I OB20
G17V I 17 + G17 I P19 + G17 I OB19

Important note: G17V 16 was excluded as according to Primer 3 the only suitable reverse primer was the G17 I P20 that had too strong hetero-dimers with the oligoblockers. Nevertheless, this allele-specific primer per se has acceptable thermodynamic values and should be hence kept as backup option in case all other allele-specific primers fail to pass the in vitro QC.

Design of the G17 Wild-Type Set

Given the proximity of the G17 position with the F25 position described in the previous chapter, one feasible option is to use the same WT system devised for F25, on RHOA second intron. A distinct WT set will be designed on RHOA first intron to ensure the reliability of the results.

RHOA G17 Wild-Type Set Candidates

TABLE 20
Evaluation of all thermodynamic parameters considered to select appropriate
oligonucleotides for the RHOA G17 wild-type set. Accepted values are presented with
white background.

| Primer | | | Tm | % | GC% | | | | Validated |
|---|---|---|---|---|---|---|---|---|---|
| name | Sequence | Length | (° C.) | GC | tail | self-dimers | Hairpins | Heterodimers | (Y/N) |
| G17 WTL | GACACATGCTTTGGTAACC | 19 | 56 | 47.4 | 57.1 | -2.17 | none | -1.81 | Y |
| G17 WTR | CTTTTGGTGCCAGGTGGA | 18 | 56 | 55.6 | 57.1 | -1.81 | none | | Y |

| | | | | | | | | |
|---|---|---|---|---|---|---|---|---|
| Accepted values for the parameters | 16-22 | 55-60° C. | 40-60% | >25% | >-4 Kcal/ | >-3 Kcal/ | >-4 Kcal/ | Y |
| in analysis (wild-type primer set) | bp | ΔTm with | | | mol | mol | mol | |
| | | paired | | | | | | |
| | | primer <5° C. | | | | | | |

Blat Alignment of the Validated WT Primer Pairs

The Blat local alignment (FIG. 9) highlighted the presence of a partial secondary homology region that spans the G17 WT L primer binding site. Nevertheless, there are 4 to mismatches between the secondary target on chromosome 15 and the selected primer.

Moreover, no homology is reported for the G17 WT R primer binding site, so no secondary product is expected to be produced. This primer combination can hence be validated.

The oligonucleotide candidates of the RHOA G17 WT set meet the thermodynamic criteria required to ensure absence of non-specific amplification and to improve the amplification efficiency.

RHOA C16STOP

The inventors have designed specific sets of primers allowing improved detection of the presence (or absence, or frequency) of such a mutation from a sample containing cfNA or genomic DNA.

More specifically, in a preferred embodiment, the mutation is RHOA C16*, where the cysteine to stop codon change is due to a non-sense single point mutation at position chr3: 49412975 (human assembly GRCh37/hg19) where the wild-type allele is an adenine (A) and the mutant allele is a thymidine (T). The first set of reagents for detecting the RHOA C16* mutation comprises a mutation-specific primer selected from SEQ ID NO: 72-85, a paired primer selected from SEQ ID NO: 53-59, 86-88, and an oligoblocker selected from SEQ ID NO: 60-69. Most preferably, the mutation-specific primer is selected from SEQ ID NO: 75-78, 83-85, the paired primer is selected from SEQ ID NO: 53-54, 58-59, 86-88 and the oligoblocker is selected from SEQ ID NO: 62-64, 67-69. In another embodiment, a different paired primer can be chosen from ANY consecutive DNA sequence of size comprised between 15 and 25 base pairs selected in the following regions: chr3: 49412740-49412950 and/or chr3: 49413000-49413210 (human assembly GRCh37/hg19). A different oligoblocker can be chosen from ANY consecutive DNA sequence of size comprised between 15 and 25 base pairs selected in the following regions: chr3: 49412950-49413000 (human assembly GRCh37/hg19) with the fundamental prerequisite that the position chr3: 49412975 must be included in the selected oligoblocker sequence. In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 70 and a second primer SEQ ID NO: 71.

Primer Design for the Allele-Specific RHOA C16* System

The allele-specific primers considered in the design of this invention and their thermodynamic characteristics are listed in Table 21:

TABLE 21
List of all the RHOA C16* allele-specific primers considered in the design of
this invention and their thermodynamic characteristics. Accepted values are presented
with white background. Borderline values are presented with light grey background and
non-acceptable values are presented with dark grey background. In the eventuality that
one of the parameters in analysis falls outside the accepted range indicated in the last
row the thermodynamic analysis is discontinued and the primer is discarded.

| | | | | | | | |
|---|---|---|---|---|---|---|---|
| Primer name | Sequence | Length | Tm (° C.) | GC% | GC% tail | self-dimers | Hairpins |

Conventional configuration

| | | | | | | | |
|---|---|---|---|---|---|---|---|
| C16*22 | TATGAGCAAGCATGTCTTTCCT | 22 | 62 | | | | |
| C16*21 | ATGAGCAAGCATGTCTTTCCT | 21 | 60 | | | | |
| C16*20 | TGAGCAAGCATGTCTTTCCT | 20 | 58 | | | | |
| C16*19 | GAGCAAGCATGTCTTTCCT | 19 | 56 | 47.4 | 42.9 | -2.17 | -0.21 |
| C16*18 | AGCAAGCATGTCTTTCCT | 18 | 52 | 44.4 | 42.9 | -2.17 | -0.21 |
| C16*17 | GCAAGCATGTCTTTCCT | 17 | 50 | 47.1 | 42.9 | -2.17 | -0.21 |
| C16*16 | CAAGCATGTCTTTCCT | 16 | 46 | 43.8 | 42.9 | -2.17 | -0.21 |

Inverse configuration

| | | | | | | | |
|---|---|---|---|---|---|---|---|
| C16* \| 22 | GATTGTTGGTGATGGAGCCTGA | 22 | 66 | | | | |
| C16* \| 21 | ATTGTTGGTGATGGAGCCTGA | 21 | 62 | | | | |
| C16* \| 20 | TTGTTGGTGATGGAGCCTGA | 20 | 60 | | | | |
| C16* \| 19 | TGTTGGTGATGGAGCCTGA | 19 | 58 | | | | |
| C16* \|18 | GTTGGTGATGGAGCCTGA | 18 | 56 | 55.6 | 57.1 | none | none |
| C16* \| 17 | TTGGTGATGGAGCCTGA | 17 | 52 | 52.9 | 57.1 | none | none |
| C16* \|16 | TGGTGATGGAGCCTGA | 16 | 50 | 56.3 | 57.1 | none | none |

| | | | | | | |
|---|---|---|---|---|---|---|
| Accepted values for the parameters | 16-22 | 45-55° | 40-60% | >25% | >-4 Kcal/ | >-3 Kcal/ |
| in analysis (allele-specific primer) | bp | C. | | | mol | mol |

Paired Wild-Type Primer

Paired Wild Type Primers in Conventional Configuration

As paired wild type primer the first attempt will be evaluate the thermodynamic characteristics of the paired primers for the G17V assay that passed the QC step, as it would enable the use of the same primers for multiple assays, to reduce the number of variables.

Note that G17 P21 and G17 P20 present unacceptable heterodimers with the G17 oligoblocker, that will be as well evaluated as compatible oligoblocker for the C16* assay. Hence these combinations will be temporarily discarded.

It should be noted that the Tm of G17 P18 is on the lower side (˜50 C). For this reason, some extra paired primers were designed (C16* P20 and G16* P21).

TABLE 22
List of all the RHOA C16* paired primers in conventional configuration
considered in the design of this invention and their thermodynamic characteristics.
Accepted values are presented with white background and borderline values are
presented with light grey background. In the eventuality that one of the parameters in
analysis falls outside the accepted range indicated in the last row the thermodynamic
analysis is discontinued and the primer is discarded.

| | Allele | | | | | | | | | | | Off target |
|---|---|---|---|---|---|---|---|---|---|---|---|---|
| Allele- | specific | Paired | | | | | | | | | | with wt |
| specific | Primer Tm | WT primer | Paired | | Amplicon | Tm | | GC% | self- | | Off | allele- |
| Primer | (Primer3) | name | WT primer sequence | Length | size | (° C.) | GC% | tail | dimers | Hairpins | target? | specific? |
| C16*19 | 55.49 | G17 P19 | TGTGTTTTGTGTTTCAGCA | 19 | 85 | 53.85 | 36.84 | 42.9 | none | none | Just paired | no |
| | | | | | | | | | | | primer | |
| C16*18 | 54 | G17 P19 | TGTGTTTTGTGTTTCAGCA | 19 | 84 | 53.85 | 36.84 | 42.9 | none | none | Just paired | no |
| | | | | | | | | | | | primer | |
| C16*17 | 52.07 | G17 P19 | TGTGTTTTGTGTTTCAGCA | 19 | 83 | 53.85 | 36.84 | 42.9 | none | none | Just paired | no |
| | | | | | | | | | | | primer | |
| C16*16 | 47.67 | G17 P19 | TGTGTTTTGTGTTTCAGCA | 19 | 82 | 53.85 | 36.84 | 42.9 | none | none | Just paired | no |
| | | | | | | | | | | | primer | |
| C16*19 | 55.49 | G17 P18 | GTGTTTTGTGTTTCAGCA | 18 | 84 | 51.7 | 38.9 | 42.9 | none | none | Just paired | no |
| | | | | | | | | | | | primer | |
| C16*18 | 54 | G17 P18 | GTGTTTTGTGTTTCAGCA | 18 | 83 | 51.7 | 38.9 | 42.9 | none | none | Just paired | no |
| | | | | | | | | | | | primer | |
| C16*17 | 52.07 | G17 P18 | GTGTTTTGTGTTTCAGCA | 18 | 82 | 51.7 | 38.9 | 42.9 | none | none | Just paired | no |
| | | | | | | | | | | | primer | |
| C16*16 | 47.67 | G17 P18 | GTGTTTTGTGTTTCAGCA | 18 | 81 | 51.7 | 38.9 | 42.9 | none | none | Just paired | no |
| | | | | | | | | | | | primer | |
| C16*19 | 55.49 | C16* P20 | GTGTTTTGTGTTTCAGCAAT | 20 | 84 | 53.47 | 35 | 42.9 | -0.69 | -0.57 | Just paired | no |
| | | | | | | | | | | | primer | |
| C16*18 | 54 | C16* P20 | GTGTTTTGTGTTTCAGCAAT | 20 | 83 | 53.47 | 35 | 42.9 | -0.69 | -0.57 | Just paired | no |
| | | | | | | | | | | | primer | |
| C16*17 | 52.07 | C16* P20 | GTGTTTTGTGTTTCAGCAAT | 20 | 82 | 53.47 | 35 | 42.9 | -0.69 | -0.57 | Just paired | no |
| | | | | | | | | | | | primer | |
| C16*16 | 47.67 | C16* P20 | GTGTTTTGTGTTTCAGCAAT | 20 | 81 | 53.47 | 35 | 42.9 | -0.69 | -0.57 | Just paired | no |
| | | | | | | | | | | | primer | |
| C16*19 | 55.49 | C16* P21 | GTGTTTTGTGTTTCAGCAATG | 21 | 84 | 55.09 | 38.1 | 42.9 | -0.69 | -0.57 | Just paired | no |
| | | | | | | | | | | | primer | |
| C16*18 | 54 | C16* P21 | GTGTTTTGTGTTTCAGCAATG | 21 | 83 | 55.09 | 38.1 | 42.9 | -0.69 | -0.57 | Just paired | no |
| | | | | | | | | | | | primer | |
| C16*17 | 52.07 | C16* P21 | GTGTTTTGTGTTTCAGCAATG | 21 | 82 | 55.09 | 38.1 | 42.9 | -0.69 | -0.57 | Just paired | no |
| | | | | | | | | | | | primer | |
| C16*16 | 47.67 | C16* P21 | GTGTTTTGTGTTTCAGCAATG | 21 | 81 | 55.09 | 38.1 | 42.9 | -0.69 | -0.57 | Just paired | no |
| | | | | | | | | | | | primer | |

| | | | | | | | | | |
|---|---|---|---|---|---|---|---|---|---|
| Accepted values for the parameters in analysis | 16-25 | <100 bp | ΔTm with | 40-60% | >25% | >-4 Kcal/ | -3 Kcal/ | no | no |
| (paired WT primer) | bp | | paired | | | mol | mol | | |
| | | | primer | | | | | | |
| | | | <5° C. | | | | | | |

Blat Alignment of the Validated Primer Pairs in Conventional Sense

In this case just the primer pair sets generating the longest amplicons have been considered for the Blat analysis (FIG. 10), as all shorter primers to generate primer products contained within the amplicons here presented.

>C16*19 + G17_P19
SEQ ID No: 165
GAGCAAGCATGTCTTTCCTcaggctccatcaccaacaatcaccagtttc
ttccggatggcagccatTGCTGAAACACAAAACACA
>C16*_19 + C16*P21
SEQ ID No: 166
GAGCAAGCATGTCTTTCCTcaggctccatcaccaacaatcaccagtttc
ttccggatggcagcCATTGCTGAAACACAAAACAC

When in conventional configuration, the paired primers hit a secondary region of homology on chromosome 6. Nevertheless, as indicated from the characteristics of the reported homology (FIG. 9), the homology region does not cover the landing site of the forward primer, so that no amplification outside the true target region will be possible. These primer combinations can hence be accepted and analysed further.

All primer pairs in conventional configuration are expected to produce just one single amplicon at the expected chromosomal position. No other homologies are reported.

All tested combinations in conventional configuration meet the thermodynamic criteria required.

Paired Wild Type Primers in Inverse Configuration

As for the paired wild-type primers in conventional configuration, the first attempt will be evaluating the thermodynamic characteristics of the paired primers for the G17V assay that passed the QC step, as it would enable the use of the same primers for multiple assays, to reduce the number of variables. It should be noted that the paired primer G17 I P20 generated excessive hetero-dimers with all selected oligoblockers and was hence excluded from the analysis. A further attempt extending the primer in the opposite direction should be made.

TABLE 23
List of all the RHOA C16* paired primers in inverse configuration considered
in the design of this invention and their thermodynamic characteristics. Accepted
values are presented with white background and borderline values are presented with
light grey background. In the eventuality that one of the parameters in analysis falls
outside the accepted range indicated in the last row the thermodynamic analysis is
discontinued and the primer is discarded.

| | allele- | | | | | | | | | | | |
|---|---|---|---|---|---|---|---|---|---|---|---|---|
| Allele- | specific | Paired | | | | | | | | | | Off target |
| specific | Primer Tm | WT primer | Paired | | Amplicon | Tm | | GC% | self- | | off | with wt |
| Primer | (Primer3) | name | WT primer sequence | Length | size | (° C.) | GC% | tail | dimers | Hairprins | target? | allele-specific? |
| C16* \| 18 | 56.53 | G17 \| P18 | TTCTCAAACACTGTGGGC | 18 | 92 | 55.06 | 50 | 71.4 | -0.08 | none | no | no |
| | | G17 \| P19 | GAACTGGTCCTTGCTGAAG | 19 | 60 | 55.84 | 52.63 | 57.1 | -0.33 | -0.21 | no | no |
| C16* \| 17 | 54.66 | G17 \| P18 | TTCTCAAACACTGTGGGC | 18 | 91 | 55.06 | 50 | 71.4 | -0.08 | none | no | no |
| | | G17 \| P19 | GAACTGGTCCTTGCTGAAG | 19 | 59 | 55.84 | 52.63 | 57.1 | -0.33 | -0.21 | no | no |
| C16* \| 16 | 53.55 | G17 \| P18 | TTCTCAAACACTGTGGGC | 18 | 90 | 55.06 | 50 | 71.4 | -0.08 | none | no | no |
| | | G17 \| P19 | GAACTGGTCCTTGCTGAAG | 19 | 58 | 55.84 | 52.63 | 57.1 | -0.33 | -0.21 | no | no |
| C16* \| 18 | 56.53 | C16* \| P20 | ATAGTTCTCAAACACTGTGG | 20 | 96 | 52.84 | 40 | 57.1 | -0.08 | 0.39 | no | no |
| C16* \| 17 | 54.66 | C16* \| P20 | ATAGTTCTCAAACACTGTGG | 20 | 95 | 52.84 | 40 | 57.1 | -0.08 | 0.39 | no | no |
| C16* \| 16 | 53.55 | C16* \| P20 | ATAGTTCTCAAACACTGTGG | 20 | 94 | 52.84 | 40 | 57.1 | -0.08 | 0.39 | no | no |

Blat alignment of the validated primer pairs in inverse configuration

>C16*_I_18 + G17_I_P18/92 bp
SEQ ID No: 167
TTCTCAAACACTGTGGGCacatacacctctgggaactggtccttgctga
agactatgagcaagcatgtctttccTCAGGCTCCATCACCAAC
>C16*_I_18 + G17_I_P19/60 bp
SEQ ID No: 168
GAACTGGTCCTTGCTGAAGactatgagcaagcatgtctttccTCAGGCT
CCATCACCAAC
>C16*_I_17 + G17_I_P18/91 bp
SEQ ID No: 169
TTCTCAAACACTGTGGGCacatacacctctgggaactggtccttgctga
agactatgagcaagcatgtctttccTCAGGCTCCATCACCAA
>C16*_I_17 + G17_I_P19/59 bp
SEQ ID No: 170
GAACTGGTCCTTGCTGAAGactatgagcaagcatgtctttccTCAGGCT
CCATCACCAA
>C16*_I_16 + G17_I_P18/90 bp
SEQ ID No: 171
TTCTCAAACACTGTGGGCacatacacctctgggaactggtccttgctga
agactatgagcaagcatgtctttccTCAGGCTCCATCACCA
>C16*_I_16 + G17_I_P19/58 bp
SEQ ID No: 172
GAACTGGTCCTTGCTGAAGactatgagcaagcatgtctttccTCAGGCT
CCATCACCA
>C16*_I_18 + C16*_I_P20_96 bp (C16* I17 and C16*
I16 are contained within the following product)

SEQ ID No: 173
ATAGTTCTCAAACACTGTGGgcacatacacctctgggaactggtccttg
ctgaagactatgagcaagcatgtctttccTCAGGCTCCATCACCAAC

The paired primer G17 I P19 generates an homology region on chromosome 6 (FIG. 12). Nevertheless, the allele-specific primer is specific so no non-specific product should be amplified.

All other combinations are perfectly fine.

By working in inverse configuration, it is possible to observe a homology region on chromosome 6 when using the paired wt primer G17 I P19 (FIG. 13). It is important to notice that the allele-specific primer is not included in the homology region so this is unlikely to generate non-specific products. Nevertheless, this aspect should be flagged during the in vitro testing, aimed at excluding any risk of secondary amplification.

Wet-lab validation will be performed preferentially on all thermodynamically suitable primer pairs

RHOA C16 Oligoblocker Candidates in Conventional and Inverse Configuration

As for the paired wild-type primers in conventional and inverse configuration, the first attempt will be evaluating the thermodynamic characteristics of the oligoblockers designed for the G17V assay that passed the QC step, as it would enable the use of the same oligoblocker for multiple assays, to reduce the number of variables.

The position of the WT allele on the oligoblocker is indicated in bold and underlined in the following table:

TABLE 24
List of all potential oligoblockers for the RHOA G17 and C16 codon, in
conventional and inverse configuration, considered in the design of this invention and
their thermodynamic characteristics. Note that Tm are calculated using the
Oligoanalyzer ® software. Accepted values are presented with white background.
Borderline values are presented with light grey background and non-acceptable values
are presented with dark grey background. In the eventuality that one of the parameters
in analysis falls outside the accepted range indicated in the last row the thermodynamic
analysis is discontinued and the oligoblocker is discarded

| Oligoblocker | | | Tm | | GC% | | |
|---|---|---|---|---|---|---|---|
| name | Sequence | Length | (°C.) | GC% | tail | self-dimers | Hairpins |
| G17 OB23 | CATGTCTTTCCACAGGCTCCATC | 23 | 70 | | | | |
| G17 OB22 | ATGTCTTTCCACAGGCTCCATC | 22 | 66 | | | | |
| G17 OB21 | ATGTCTTTCCACAGGCTCCAT | 21 | 62 | 47.6 | 57.1 | -0.22 | -0.1 |
| G17 OB20 | ATGTCTTTCCACAGGCTCCA | 20 | 60 | 50 | 71.4 | -0.08 | 0.04 |
| G17 OB19 | ATGTCTTTCCACAGGCTCC | 19 | 58 | 52.6 | 71.4 | -0.08 | 0.04 |
| Oligoblocker | | | Tm | | GC% | | |
| name | Sequence | Length | (° C.) | GC% | tail | self-dimers | Hairpins |
| G17 \| OB23 | ATGGAGCCTGTGGAAAGACATGC | 23 | 70 | | | | |
| G17 \| OB22 | ATGGAGCCTGTGGAAAGACATG | 22 | 66 | | | | |
| G17 \| OB21 | ATGGAGCCTGTGGAAAGACAT | 21 | 62 | 47.6 | 28.6 | -0.22 | -0.1 |
| G17 \| OB20 | ATGGAGCCTGTGGAAAGACA | 20 | 60 | 50 | 28.6 | -0.08 | 0.04 |
| G17 \| OB19 | TGGAGCCTGTGGAAAGACA | 19 | 58 | 52.6 | 28.6 | -0.08 | 0.04 |

| | | | | | | |
|---|---|---|---|---|---|---|
| Accepted values for the parameters | 19-23 | ~60° | 40-60% | >25% | >-4 Kcal/ | >-3 Kcal/ |
| in analysis (Oligoblocker) | bp | C. | | | mol | mol |

Final Thermodynamic Analysis on All Candidate Primers and Oligoblockers

Heterodimers combinations to consider for all the primers/oligoblocker candidates that met the thermodynamic criteria described in the previous sections:
Allele-specific primer+paired WT primer
Allele-specific primer+oligoblocker
Paired WT primer+oligoblocker

Thermodynamic analysis in conventional configuration

TABLE 25
Evaluation of heterodimers presence for all oligonucleotide candidates
in conventional configuration evaluated for the allele-specific
system. Accepted values are presented with white background and
borderline values are presented with light grey background.

| Oligonucleotide combination | ΔG | Validated? |
|---|---|---|
| (conventional) | (Kcal/mol) | Y/N |
| C16*19 + G17 P19 | −0.69 | Y |
| C16*19 + G17 P18 | −0.69 | Y |
| C16*19 + C16* P20 | −0.69 | Y |
| C16*19 + C16* P21 | −0.69 | Y |
| C16*18 + G17 P19 | −0.69 | Y |
| C16*18 + G17 P18 | −0.69 | Y |
| C16*18 + C16* P20 | −0.69 | Y |
| C16*18 + C16* P21 | −0.69 | Y |
| C16*17 + G17 P19 | −0.69 | Y |
| C16*17 + G17 P18 | −0.69 | Y |
| C16*17 + C16* P20 | −0.69 | Y |
| C16*17 + C16* P21 | −0.69 | Y |
| C16*16 + G17 P19 | −0.69 | Y |
| C16*16 + G17 P18 | −0.69 | Y |
| C16*16 + C16* P20 | −0.69 | Y |
| C16*16 + C16* P21 | −0.69 | Y |
| C16*19 + G17 OB21 | −3.1 | Borderline |
| C16*19 + G17 OB20 | −3.1 | Borderline |
| C16*19 + G17 OB19 | −3.1 | Borderline |
| C16*18 + G17 OB21 | −1.53 | Y |
| C16*18 + G17 OB20 | −1.53 | Y |
| C16*18 + G17 OB19 | −1.53 | Y |
| C16*17 + G17 OB21 | −1.53 | Y |
| C16*17 + G17 OB20 | −1.53 | Y |
| C16*17 + G17 OB19 | −1.53 | Y |
| C16*16 + G17 OB21 | −1.53 | Y |
| C16*16 + G17 OB20 | −1.53 | Y |
| C16*16 + G17 OB19 | −1.53 | Y |
| G17 P19 + G17 OB21 | −2.04 | Y |
| G17 P19 + G17 OB20 | −2.04 | Y |
| G17 P19 + G17 OB19 | −2.04 | Y |
| G17 P18 + G17 OB21 | −2.04 | Y |
| G17 P18 + G17 OB20 | −2.04 | Y |
| G17 P18 + G17 OB19 | −2.04 | Y |
| C16* P20 + G17 OB21 | −2.04 | Y |
| C16* P20 + G17 OB20 | −2.04 | Y |
| C16* P20 + G17 OB19 | −2.04 | Y |
| C16* P21 + G17 OB21 | −2.04 | Y |
| C16* P21 + G17 OB20 | −2.04 | Y |
| C16* P21 + G17 OB19 | −2.04 | Y |
| Accepted values for the | >−4 Kcal/mol | Y |
| parameters in analysis | | |

All the oligonucleotide candidates in conventional configuration of the table above meet the thermodynamic criteria required to ensure absence of non-specific amplification and to improve the amplification efficiency.

It should be reported and considered in the following in vitro validation phases that the C16* 19 allele-specific primers form borderline hetero-dimers with the oligoblockers in conventional configuration. This cannot be avoided given the mutation position.

Thermodynamic analysis in inverse configuration

TABLE 25
Evaluation of heterodimers presence for all oligonucleotide candidates
in inverse configuration evaluated for the allele-specific system.
Accepted values are presented with white background and borderline
values are presented with light grey background.

| Oligonucleotide combination | ΔG | Validated? |
|---|---|---|
| (inverse) | (Kcal/mol) | Y/N |
| C16* I 18 + G17 I P18 | −3 | borderline |
| C16* I 18 + G17 I P19 | −1.53 | Y |
| C16* I 17 + G17 I P18 | −3 | borderline |
| C16* I 17 + G17 I P19 | −1.53 | Y |
| C16* I 16 + G17 I P18 | −3 | borderline |
| C16* I 16 + G17 I P19 | −1.53 | Y |
| C16* I 18 + C16* I P20 | −0.69 | Y |
| C16* I 17 + C16* I P20 | −0.69 | Y |
| C16* I 16 + C16* I P20 | −0.31 | Y |
| C16* I 18 + G17 I OB21 | −0.22 | Y |
| C16* I 18 + G17 I OB20 | none | Y |
| C16* I 18 + G17 I OB19 | none | Y |
| C16* I 17 + G17 I OB21 | −0.22 | Y |
| C16* I 17 + G17 I OB20 | none | Y |
| C16* I 17 + G17 I OB19 | none | Y |
| C16* I 16 + G17 I OB21 | −0.22 | Y |
| C16* I 16 + G17 I OB20 | none | Y |
| C16* I 16 + G17 I OB19 | none | Y |
| G17 I P18 + G17 I OB21 | −3 | Borderline |
| G17 I P18 + G17 I OB20 | −3 | Borderline |
| G17 I P18 + G17 I OB19 | −3 | Borderline |
| G17 I P19 + G17 I OB21 | −1.53 | Y |
| G17 I P19 + G17 I OB20 | −1.53 | Y |
| G17 I P19 + G17 I OB19 | −1.53 | Y |
| C16* I P20 + G17 I OB21 | −0.31 | Y |
| C16* I P20 + G17 I OB20 | −0.31 | Y |
| C16* I P20 + G17 I OB19 | −0.31 | Y |
| Accepted values for the | >−4 Kcal/mol | Y |
| parameters in analysis | | |

All the oligonucleotide candidates in inverse configuration of the table above meet the thermodynamic criteria required to ensure absence of non-specific amplification and to improve the amplification efficiency.

Priority should be given to the combinations generating less heterodimers.

Design of the C16* Wild-Type Set

Given the proximity of the C16* position with the F25 and the G17V position described in the previous chapters, the correspondent WT assays will be used in conjunction with the C16* mutation specific assay.

RHOA G14V

The inventors have designed specific sets of primers allowing improved detection of the presence (or absence, or frequency) of such a mutation from a sample containing cfNA or genomic DNA.

More specifically, in a preferred embodiment, the mutation is RHOA G14V, where the glycine to valine change is due to a single point mutation at position chr3: 49412982 (human assembly GRCh37/hg19) where the wild-type allele is a cytosine (C) and the mutant allele is an adenine (A). The first set of reagents for detecting the RHOA G14V mutation comprises a mutation-specific primer selected from SEQ ID NO: 89-102, a paired primer selected from SEQ ID NO: 53-59, 86-88, and an oligoblocker selected 30 from SEQ ID NO: 60-69, 103-112. Most preferably, the mutation-specific primer is selected from SEQ ID NO: 92-95, 99-101, the paired primer is selected from SEQ ID NO: 54, 58-59, 86-88 and the oligoblocker is selected from SEQ ID NO: 62-64, 67-69, 106-107, 111-112. In another embodiment, a different paired primer can be chosen from ANY consecutive DNA sequence of size comprised between 15 and 25 base pairs selected in the following regions: chr3: 49412747-49412957 and/or chr3: 49413007-49413217 (human assembly GRCh37/hg19). A different oligoblocker can be chosen from ANY consecutive DNA sequence of size comprised between 15 and 25 base pairs selected in the following regions: chr3: 49412957-49413007 (human assembly GRCh37/hg19) with the fundamental prerequisite that the position chr3: 49412982 must be included in the selected oligoblocker sequence. In a preferred embodiment, the method amplifies a second WT target sequence, located preferably 200-400 bp from the first mutated target sequence, using a second set of reagents. In a more preferred embodiment, the second set of reagents comprises a first primer SEQ ID NO: 70 and a second primer SEQ ID NO: 71.

Primer Design for the Allele-Specific RHOA G14V System

The allele-specific primers considered in the design of this invention and their thermodynamic characteristics are listed in Table 26:

TABLE 26
List of all the RHOA G14V allele-specific primers considered in the design of
this invention and their thermodynamic characteristics. Accepted values are presented
with white background. Borderline values are presented with light grey background and
non-acceptable values are presented with dark grey background. In the eventuality that
one of the parameters in analysis falls outside the accepted range indicated in the last
row the thermodynamic analysis is discontinued and the primer is discarded.

| | | | | | | | |
|---|---|---|---|---|---|---|---|
| Primer name | Sequence | Length | Tm (° C.) | GC% | GC% tail | self-dimers | Hairpins |

Conventional configuration

| | | | | | | | |
|---|---|---|---|---|---|---|---|
| G14V 22 | AAGCATGTCTTTCCTCAGGCTA | 22 | 64 | | | | |
| G14V 21 | AGCATGTCTTTCCTCAGGCTA | 21 | 62 | | | | |
| G14V 20 | GCATGTCTTTCCTCAGGCTA | 20 | 60 | | | | |
| G14V 19 | CATGTCTTTCCTCAGGCTA | 19 | 56 | 47.4 | 57.1 | -2.17 | none |
| G14V 18 | ATGTCTTTCCTCAGGCTA | 18 | 52 | 44.4 | 57.1 | -1.46 | none |
| G14V 17 | TGTCTTTCCTCAGGCTA | 17 | 50 | 47.1 | 57.1 | -1.46 | none |
| G14V 16 | GTCTTTCCTCAGGCTA | 16 | 48 | 50 | 57.1 | -1.46 | none |

Inverse configuration

| | | | | | | | |
|---|---|---|---|---|---|---|---|
| G14V\|22 | AACTGGTGATTGTTGGTGATGT | 22 | 62 | | | | |
| G14V\|21 | ACTGGTGATTGTTGGTGATGT | 21 | 60 | | | | |
| G14V\|20 | CTGGTGATTGTTGGTGATGT | 20 | 58 | | | | |
| G14V\|19 | TGGTGATTGTTGGTGATGT | 19 | 54 | 42.1 | 42.9 | none | none |
| G14V\|18 | GGTGATTGTTGGTGATGT | 18 | 52 | 44.4 | 42.9 | none | none |
| G14V\|17 | GTGATTGTTGGTGATGT | 17 | 48 | 41.2 | 42.9 | none | none |
| G14V\|16 | TGATTGTTGGTGATGT | 16 | 44 | | | | |

| | | | | | | |
|---|---|---|---|---|---|---|
| Accepted values for the parameters | 16-22 | 45-55°C | 40-60% | >25% | >-4 Kcal/ | >-3 Kcal/ |
| in analysis (allele-specific primer) | bp | | | | mol | mol |

Paired Wild-Type Primer

Paired Wild Type Primers in Conventional Configuration

As paired wild type primer the first attempt will be evaluate the thermodynamic characteristics of the paired primers for the G17V and C16* assay that passed the QC step, as it would enable the use of the same primers for multiple assays, to reduce the number of variables.

Note that G17 P21 and G17 P20 present unacceptable heterodimers with the G17 oligoblocker, that will be as well evaluated as compatible oligoblocker for the G14V assay. Hence these combinations will be temporarily discarded. Nevertheless, in case a new oligoblocker specific for G14V will be evaluated, these two combinations will be reconsidered.

TABLE 27
List of all the RHOA G14V paired primers in conventional configuration
considered in the design of this invention and their thermodynamic characteristics.
Accepted values are presented with white background and borderline values are

| | Allele- | | | | | | | | | | | Off target |
|---|---|---|---|---|---|---|---|---|---|---|---|---|
| Allele- | specific | Paired | | | | | | | | | | with wt |
| specific | Primer Tm | WT primer | Paired | | Amplicon | Tm | | GC% | self- | | | allele- |
| Primer | (PrimerS) | name | WT primer sequence | Length | size | (° C.) | GC% | tail | dimers | Hairpins | Off target? | specific? |
| G14V 19 | 53.84 | G17 P19 | TGTGTTTTGTGTTTCAGCA | 19 | 78 | 53.85 | 36.84 | 42.9 | none | none | Just paired | no |
| | | | | | | | | | | | primer | |
| G14V 18 | 51.87 | G17 P19 | TGTGTTTTGTGTTTCAGCA | 19 | 77 | 53.85 | 36.84 | 42.9 | none | none | Just paired | no |
| | | | | | | | | | | | primer | |
| G14V 17 | 50.96 | G17 P19 | TGTGTTTTGTGTTTCAGCA | 19 | 76 | 53.85 | 36.84 | 42.9 | none | none | Just paired | no |
| | | | | | | | | | | | primer | |
| G14V 16 | 48.36 | G17 P19 | TGTGTTTTGTGTTTCAGCA | 19 | 75 | 53.85 | 36.84 | 42.9 | none | none | Just paired | no |
| | | | | | | | | | | | primer | |
| G14V 19 | 53.84 | G17 P18 | GTGTTTTGTGTTTCAGCA | 18 | 77 | 51.7 | 38.9 | 42.9 | none | none | Just paired | no |
| | | | | | | | | | | | primer | |
| G14V 18 | 51.87 | G17 P18 | GTGTTTTGTGTTTCAGCA | 18 | 76 | 51.7 | 38.9 | 42.9 | none | none | Just paired | no |
| | | | | | | | | | | | primer | |
| G14V 17 | 50.96 | G17 P18 | GTGTTTTGTGTTTCAGCA | 18 | 75 | 51.7 | 38.9 | 42.9 | none | none | Just paired | no |
| | | | | | | | | | | | primer | |
| G14V 16 | 48.36 | G17 P18 | GTGTTTTGTGTTTCAGCA | 18 | 74 | 51.7 | 38.9 | 42.9 | none | none | Just paired | no |
| | | | | | | | | | | | primer | |
| G14V 19 | 53.84 | C16* P20 | GTGTTTTGTGTTTCAGCAAT | 20 | 77 | 53.47 | 35 | 42.9 | -0.69 | -0.57 | Just paired | no |
| | | | | | | | | | | | primer | |
| G14V 18 | 51.87 | C16* P20 | GTGTTTTGTGTTTCAGCAAT | 20 | 76 | 53.47 | 35 | 42.9 | -0.69 | -0.57 | Just paired | no |
| | | | | | | | | | | | primer | |
| G14V 17 | 50.96 | C16* P20 | GTGTTTTGTGTTTCAGCAAT | 20 | 75 | 53.47 | 35 | 42.9 | -0.69 | -0.57 | Just paired | no |
| | | | | | | | | | | | primer | |
| G14V 16 | 48.36 | C16* P20 | GTGTTTTGTGTTTCAGCAAT | 20 | 74 | 53.47 | 35 | 42.9 | -0.69 | -0.57 | Just paired | no |
| | | | | | | | | | | | primer | |
| G14V 19 | 53.84 | C16* P21 | GTGTTTTGTGTTTCAGCAATG | 21 | 77 | 55.09 | 38.1 | 42.9 | -0.69 | -0.57 | Just paired | no |
| | | | | | | | | | | | primer | |
| G14V 18 | 51.87 | C16* P21 | GTGTTTTGTGTTTCAGCAATG | 21 | 76 | 55.09 | 38.1 | 42.9 | -0.69 | -0.57 | Just paired | no |
| | | | | | | | | | | | primer | |
| G14V 17 | 50.96 | C16* P21 | GTGTTTTGTGTTTCAGCAATG | 21 | 75 | 55.09 | 38.1 | 42.9 | -0.69 | -0.57 | Just paired | no |
| | | | | | | | | | | | primer | |
| G14V 16 | 48.36 | C16* P21 | GTGTTTTGTGTTTCAGCAATG | 21 | 74 | 55.09 | 38.1 | 42.9 | -0.69 | -0.57 | Just paired | no |
| | | | | | | | | | | | primer | |

| | | | | | | | | | |
|---|---|---|---|---|---|---|---|---|---|
| Accepted values for the parameters in analysis | 16-25 | <100 | ΔTm with | 40-60% | >25% | >-4 Kcal/ | >-3 Kcal/ | no | no |
| (paired WT primer) | bp | bp | paired | | | mol | mol | | |
| | | | primer | | | | | | |
| | | | <5° C. | | | | | | |

presented with light grey background. In the eventuality that one of the parameters in analysis falls outside the accepted range indicated in the last row the thermodynamic analysis is discontinued and the primer is discarded.

Blat Alignment of the Validated Primer Pairs in Conventional Sense

In this case just the primer pair sets generating the longest amplicons have been considered for the Blat analysis, as all shorter primers to generate primer products contained within the amplicons here presented.

>G14V19 + G17_P19
SEQ ID No: 174
AAGCATGTCTTTCCTCAGGCTAcatcaccaacaatcaccagtttcttc
cggatggcagccatTGCTGAAACACAAAACACA
>G14V + C16*P21
SEQ ID No: 175
AAGCATGTCTTTCCTCAGGCTAcatcaccaacaatcaccagtttcttc
cggatggcagcCATTGCTGAAACACAAAACAC

When in conventional configuration, the paired primers hit a secondary region of homology on chromosome 6 and on chromosome 8. Nevertheless, as indicated from the characteristics of the reported homology (FIG. 14), the homology region does not cover the landing site of the forward primer, so that no amplification outside the true target region will be possible (FIG. 15). These primer combinations can hence be accepted and analysed further.

All primer pairs in conventional configuration are expected to produce just one single amplicon at the expected chromosomal position. No other homologies are reported.

All tested combinations in conventional configuration meet the thermodynamic criteria required.

Paired Wild Type Primers in Inverse Configuration

As for the paired wild-type primers in conventional configuration, the first attempt will be evaluating the thermodynamic characteristics of the paired primers for the G17V and the C1*assays that passed the QC step, as it would enable the use of the same primers for multiple assays, to reduce the number of variables. It should be noted that the paired primer G17 I P20 generated excessive hetero-dimers with the selected oligoblockers for G17V and C16* and was excluded from the analysis. In case a new oligoblocker will be proposed for the G14V assay, the G17 I P20 primer will be re-analysed.

TABLE 28
List of all the RHOA G14V paired primers in inverse configuration considered
in the design of this invention and their thermodynamic characteristics. Accepted
values are presented with white background and borderline values are presented with
light grey background. In the eventuality that one of the parameters in analysis falls
outside the accepted range indicated in the last row the thermodynamic analysis is
discontinued and the primer is discarded.

| | Allele- | | | | | | | | | | | |
|---|---|---|---|---|---|---|---|---|---|---|---|---|
| Allele- | specific | Paired | | | | | | | | | | Off target |
| specific | Primer Tm | WT primer | Paired | | Amplicon | Tm | | GC% | self- | | Off | with wt allele- |
| Primer | (Primer3) | name | WT primer sequence | Length | size | (° C.) | GC% | tail | dimers | Hairpins | target? | specific? |
| G14V \| 19 | 54.56 | G17 \| P18 | TTCTCAAACACTGTGGGC | 18 | 100 | 55.06 | 50 | 71.4 | -0.08 | none | no | no |
| | | G17 \| P19 | GAACTGGTCCTTGCTGAAG | 19 | 68 | 55.84 | 52.63 | 57.1 | -0.33 | -0.21 | no | no |
| G14V \| 18 | 54.66 | G17 \| P18 | TTCTCAAACACTGTGGGC | 18 | 99 | 55.06 | 50 | 71.4 | -0.08 | none | no | no |
| | | G17 \| P19 | GAACTGGTCCTTGCTGAAG | 19 | 67 | 55.84 | 52.63 | 57.1 | -0.33 | -0.21 | no | no |
| G14V \| 17 | 53.55 | G17 \| P18 | TTCTCAAACACTGTGGGC | 18 | 98 | 55.06 | 50 | 71.4 | -0.08 | none | no | no |
| | | G17 \| P19 | GAACTGGTCCTTGCTGAAG | 19 | 66 | 55.84 | 52.63 | 57.1 | -0.33 | -0.21 | no | no |
| G14V \| 19 | 56.53 | C16* \| P20 | ATAGTTCTCAAACACTGTGG | 20 | 104 | 52.84 | 40 | 57.1 | -0.08 | 0.39 | no | no |
| G14V \| 18 | 54.66 | C16* \| P20 | ATAGTTCTCAAACACTGTGG | 20 | 103 | 52.84 | 40 | 57.1 | -0.08 | 0.39 | no | no |
| G14 V\| 17 | 53.55 | C16* \| P20 | ATAGTTCTCAAACACTGTGG | 20 | 102 | 52.84 | 40 | 57.1 | -0.08 | 0.39 | no | no |

| | | | | | | | | | |
|---|---|---|---|---|---|---|---|---|---|
| Accepted values for the parameters in analysis | 16-25 | <100 | ΔTm with | 40-60% | >25% | >-4 Kcal/ | >-3 Kcal/ | no | no |
| (paired WT primer) | bp | bp | paired | | | mol | mol | | |
| | | | primer | | | | | | |

Blat Alignment of the Validated Primer Pairs in Inverse Configuration

>G14V_I_19 + G17_I_P18/100 bp
SEQ ID No: 176
TTCTCAAACACTGTGGGCacatacacctctgggaactggtccttgctg
aagactatgagcaagcatgtctttccacaggCtCCATCACCAACAATC
ACCA
>G14V_I_19 + G17_I_P19/68 bp
SEQ ID No: 177
GAACTGGTCCTTGCTGAAGactatgagcaagcatgtctttccacaggc
tCCATCACCAACAATCACCA
>G14V_I_19 + C16*_I_P20/104 bp
SEQ ID No: 178
ATAGTTCTCAAACACTGTGGgcacatacacctctgggaactggtcctt
gctgaagactatgagcaagcatgtctttccacaggctCCATCACCAAC
AATCACCA

Please note that the remaining primers G14V I 18 and G14V I 17 do produce shorter amplicons contained in the amplicons above. The homology level should hence be perfectly comparable.

By working in inverse configuration it is possible to observe some regions of partial homology. It is important to notice that just one out of two primer at a time is included

in the homology region so this is unlikely to generate non-specific products. Nevertheless, this aspect should be flagged during the in vitro testing, aimed at excluding any risk of secondary amplification.

RHOA G14 Oligoblocker Candidates in Conventional and Inverse Configuration

As for the paired wild-type primers in conventional and inverse configuration, the first attempt will be evaluating the thermodynamic characteristics of the oligoblockers designed for the G17V and the C16* assay that passed the QC step, as it would enable the use of the same oligoblocker for multiple assays, to reduce the number of variables. The position of the WT allele on the oligoblocker is indicated in bold and underlined in to the following figure:

TABLE 29
List of all potential oligoblockers for the RHOA G₁₇, C₁₆ and G₁₄ codon, in
conventional and inverse configuration, considered in the design of this invention and
their thermodynamic characteristics. Note that Tm are calculated using the
Oligoanalyzer® software. Accepted values are presented with white background.
Borderline values are presented with light grey background and non-acceptable values
are presented with dark grey background. In the eventuality that one of the parameters
in analysis falls outside the accepted range indicated in the last row the thermodynamic
analysis is discontinued and the oligoblocker is discarded

| Oligoblocker | | | Tm | | GC % | | |
|---|---|---|---|---|---|---|---|
| name | Sequence | Length | (° C.) | GC % | tail | self-dimers | Hairpins |
| G17 OB23 | CATGTCTTTCCACAGGCTCCATC | 23 | 70 | | | | |
| G17 OB22 | ATGTCTTTCCACAGGCTCCATC | 22 | 66 | | | | |
| G17 OB21 | ATGTCTTTCCACAGGCTCCAT | 21 | 62 | 47.6 | 57.1 | −0.22 | −0.1 |
| G17 OB20 | ATGTCTTTCCACAGGCTCCA | 20 | 60 | 50 | 71.4 | −0.08 | 0.04 |
| G17 OB19 | ATGTCTTTCCACAGGCTCC | 19 | 58 | 52.6 | 71.4 | −0.08 | 0.04 |
| G17 \| OB23 | ATGGAGCCTGTGGAAAGACATGC | 23 | 70 | | | | |
| G17 \| OB22 | ATGGAGCCTGTGGAAAGACATG | 22 | 66 | | | | |
| G17 \| OB21 | ATGGAGCCTGTGGAAAGACAT | 21 | 62 | 47.6 | 28.6 | −0.22 | −0.1 |
| G17 \| OB20 | ATGGAGCCTGTGGAAAGACA | 20 | 60 | 50 | 28.6 | −0.08 | 0.04 |
| G17 \| OB19 | TGGAGCCTGTGGAAAGACA | 19 | 58 | 52.6 | 28.6 | −0.08 | 0.04 |

| | | | | | | |
|---|---|---|---|---|---|---|
| Accepted values for the parameters | 19-23 bp | ~60° C. | 40-60% | >25% | > −4 Kcal/mol | > −3 Kcal/mol |
| in analysis (Oligoblocker) | | | | | | |

It is important to notice that the G14 codon is in this case shifted towards the 3′ end of the oligoblocker. This is normally avoided in the in silico design. Nevertheless, as two of the other assays will use this type of oligoblocker, an in vitro analysis will be performed anyway as in case of appropriate sensitivity and specificity of the common oligoblocker, its use will be favoured over the use of three distinct oligoblockers for the four mutations in analysis.

Thermodynamic Analysis on the Allele-Specific Primers and the Common Oligoblockers G17/C16/G14.

The combinations left to consider in this section to meet the thermodynamic criteria described in the previous sections are:
Allele-specific primer+oligoblocker
Allele-specific primer+paired primer

Thermodynamic analysis in conventional configuration

TABLE 30
Evaluation of heterodimers presence for all oligonucleotide candidates
in conventional configuration evaluated for the allele-specific
system. Accepted values are presented with white background and
borderline values are presented with light grey background.

| Oligonucleotide combination | ΔG | Validated? |
|---|---|---|
| (conventional) | (Kcal/mol) | Y/N |
| G14V 19 + G17 P19 | −1.53 | Y |
| G14V 19 + G17 P18 | −1.53 | Y |
| G14V 19 + C16* P20 | −1.53 | Y |
| G14V 19 + C16* P21 | −1.53 | Y |
| G14V 18 + G17 P19 | −1.53 | Y |
| G14V 18 + G17 P18 | −1.53 | Y |
| G14V 18 + C16* P20 | −1.53 | Y |
| G14V 18 + C16* P21 | −1.53 | Y |
| G14V 17 + G17 P19 | −1.53 | Y |
| G14V 17 + G17 P18 | −1.53 | Y |
| G14V 17 + C16* P20 | −1.53 | Y |
| G14V 17 + C16* P21 | −1.53 | Y |
| G14V 16 + G17 P19 | −1.53 | Y |
| G14V 16 + G17 P18 | −1.53 | Y |
| G14V 16 + C16* P20 | −1.53 | Y |
| G14V 16 + C16* P21 | −1.53 | Y |
| G14V 19 + G17 OB21 | −1.46 | Y |
| G14V 19 + G17 OB20 | −1.46 | Y |
| G14V 19 + G17 OB19 | −1.46 | Y |
| G14V 18 + G17 OB21 | −1.46 | Y |
| G14V 18 + G17 OB20 | −1.46 | Y |
| G14V 18 + G17 OB19 | −1.46 | Y |
| G14V 17 + G17 OB21 | −1.46 | Y |
| G14V 17 + G17 OB20 | −1.46 | Y |
| G14V 17 + G17 OB19 | −1.46 | Y |
| G14V 16 + G17 OB21 | −1.46 | Y |
| G14V 16 + G17 OB20 | −1.46 | Y |
| G14V 16 + G17 OB19 | −1.46 | Y |
| Accepted values for the | >−4 Kcal/mol | Y |
| parameters in analysis | | |

All the oligonucleotide candidates in conventional configuration of the table above meet the thermodynamic criteria required to ensure absence of non-specific amplification and to improve the amplification efficiency.

Thermodynamic analysis in inverse configuration

TABLE 31
Evaluation of heterodimers presence for all oligonucleotide candidates
in inverse configuration evaluated for the allele-specific system.
Accepted values are presented with white background.

| Oligonucleotide combination | ΔG | Validated? |
|---|---|---|
| (inverse) | (Kcal/mol) | Y/N |
| G14V I 19 + G17 I P18 | −2.03 | Y |
| G14V I 19 + G17 I P19 | −0.07 | Y |
| G14V I 19 + C16* I P20 | −2.03 | Y |
| G14V I 18 + G17 I P18 | −2.03 | Y |
| G14V I 18 + G17 I P19 | −0.07 | Y |
| G14V I 18 + C16* I P20 | −2.03 | Y |
| G14V I 17 + G17 I P18 | −2.03 | Y |
| G14V I 17 + G17 I P19 | −0.07 | Y |
| G14V I 17 + C16* I P20 | −2.03 | Y |
| G14V I 19 + G17 I OB21 | −1.56 | Y |
| G14V I 19 + G17 I OB20 | −0.08 | Y |
| G14V I 19 + G17 I OB19 | −0.08 | Y |
| G14V I 18 + G17 I OB21 | −1.56 | Y |
| G14V I 18 + G17 I OB20 | −0.08 | Y |
| G14V I 18 + G17 I OB19 | −0.08 | Y |
| G14V I 17 + G17 I OB21 | −1.56 | Y |
| G14V I 17 + G17 I OB20 | −0.08 | Y |
| G14V I 17 + G17 I OB19 | −0.08 | Y |
| Accepted values for the | >−4 Kcal/mol | Y |
| parameters in analysis | | |

All the oligonucleotide candidates in inverse configuration of the table above meet the thermodynamic criteria required to ensure absence of non-specific amplification and to improve the amplification efficiency.

Priority should be given to the combinations generating less heterodimers.

RHOA G14-Centred Oligoblocker

Given that the oligoblocker described in paragraph 3.5.5 contains the G14 position towards the 3′ end of the sequence, an alternative set of oligoblockers will be designed keeping the G14 codon towards the middle of the sequence, in case the in vitro validation of all the G17 oligoblockers on the G14 codon does not satisfy the correspondent quality control parameters.

TABLE 32
Table 32: List of all potential oligoblockers for the RHOA G₁₄ codon, in conventional
and inverse configuration, considered in the design of this ivention and their

| Oligoblocker | | | Tm | | GC % | | |
|---|---|---|---|---|---|---|---|
| name | Sequence | Length | (° C.) | GC % | tail | self-dimers | Hairpins |
| G14 OB23 | TTCCACAGGCTCCATCACCAACA | 23 | 70 | | | | |
| G14 OB22 | TTCCACAGGCTCCATCACCAAC | 22 | 68 | | | | |
| G14 OB21 | TCCACAGGCTCCATCACCAAC | 21 | 66 | | | | |
| G14 OB20 | TCCACAGGCTCCATCACCAA | 20 | 62 | 55 | 42.9 | none | none |
| G14 OB19 | CCACAGGCTCCATCACCAA | 19 | 60 | 57.9 | 42.9 | none | none |
| G14 \| OB23 | TGTTGGTGATGGAGCCTGTGGAA | 23 | 70 | | | | |
| G14 \| OB22 | TGTTGGTGATGGAGCCTGTGGA | 22 | 68 | | | | |
| G14 \| OB21 | GTTGGTGATGGAGCCTGTGGA | 21 | 66 | | | | |
| G14 \| OB20 | TTGGTGATGGAGCCTGTGGA | 20 | 62 | 55 | 57.1 | none | none |
| G14 \| OB19 | TTGGTGATGGAGCCTGTGG | 19 | 60 | 57.9 | 71.4 | none | none |

| | | | | | | |
|---|---|---|---|---|---|---|
| Accepted values for the parameters in | 19-23 bp | ~60° C. | 40-60% | >25% | > −4 Kcal/mol | > −3 Kcal/mol |
| analysis (Oligoblocker) | | | | | | |

thermodynamic characteristics. Note that Tm are calculated using the Oligoanalyzer® software. Accepted values are presented with white background and non-acceptable values are presented with dark grey background. In the eventuality that one of the parameters in analysis falls outside the accepted range indicated in the last row the thermodynamic analysis is discontinued and the oligoblocker is discarded

Final Thermodynamic Analysis on All Candidate Primers and the G14 Oligoblockers

Thermodynamic analysis in conventional configuration

TABLE 33
Evaluation of heterodimers presence for all oligonucleotide candidates
in conventional configuration evaluated for the allele-specific
system. Accepted values are presented with white background.

| Oligonucleotide combination | ΔG | Validated? |
|---|---|---|
| (conventional) | (Kcal/mol) | Y/N |
| G14V 19 + G14 OB20 | −1.46 | Y |
| G14V 19 + G14 OB19 | −1.46 | Y |
| G14V 18 + G14 OB20 | −1.46 | Y |
| G14V 18 + G14 OB19 | −1.46 | Y |
| G14V 17 + G14 OB20 | −1.46 | Y |
| G14V 17 + G14 OB19 | −1.46 | Y |
| G14V 16 + G14 OB20 | −1.46 | Y |
| G14V 16 + G14 OB19 | −1.46 | Y |
| G17 P19 + G14 OB20 | −2.04 | Y |
| G17 P19 + G14 OB19 | −2.04 | Y |
| G17 P18 + G14 OB20 | −2.04 | Y |
| G17 P18 + G14 OB19 | −2.04 | Y |
| C16* P20 + G14 OB20 | −2.04 | Y |
| C16* P20 + G14 OB19 | −2.04 | Y |
| C16* P21 + G14 OB20 | −2.04 | Y |
| C16* P21 + G14 OB19 | −2.04 | Y |
| Accepted values for the | >−4 Kcal/mol | Y |
| parameters in analysis | | |

All the oligonucleotide candidates in conventional configuration of the table above meet the thermodynamic criteria required to ensure absence of non-specific amplification and to improve the amplification efficiency.

Thermodynamic analysis in inverse configuration

TABLE 34
Evaluation of heterodimers presence for all oligonucleotide candidates
in inverse configuration evaluated for the allele-specific system.
Accepted values are presented with white background and borderline
values are presented with light grey background.

| | ΔG | Validated? |
|---|---|---|
| Oligonucleotide combination | (Kcal/mol) | Y/N |
| G14V I 19 + G14 I OB20 | none | Y |
| G14V I 19 + G14 I OB19 | none | Y |
| G14V I 18 + G14 I OB20 | none | Y |
| G14V I 18 + G14 I OB19 | none | Y |
| G14V I 17 + G14 I OB20 | none | Y |
| G14V I 17 + G14 I OB19 | none | Y |
| G17 I P18 + G14 I OB19 | −3 | borderline |
| G17 I P18 + G14 I OB20 | −3 | borderline |
| G17 I P19 + G14 I OB19 | −1.53 | Y |
| G17 I P19 + G14 I OB20 | −1.53 | Y |
| C16* I P20 + G14 I OB20 | −0.69 | Y |
| C16* I P20 + G14 I OB19 | −0.69 | Y |
| Accepted values for the | >−4 Kcal/mol | Y |
| parameters in analysis | | |

All the oligonucleotide candidates in inverse configuration of the table above meet the thermodynamic criteria required to ensure absence of non-specific amplification and to improve the amplification efficiency.

Priority should be given to the combinations generating less heterodimers. In particular, the reverse primer G17 I P18 should be used just if the other combinations do not pass the in vitro QC steps.

Design of the C14 Wild-Type Set

Given the proximity of the C14 position with the C16* and the G17V position described in the previous chapters, the correspondent WT assays will be used in conjunction with the C16* mutation specific assay.

Probe-Based A-TAG Assays

RHOA F25L

The inventors have designed specific sets of primers and fluorescent probes allowing improved detection of the presence (or absence, or frequency) of such a mutation from a sample containing cfNA or genomic DNA.

More specifically, in a preferred embodiment, the mutation is RHOA F25L, where the phenylalanine to leucine change is due to a single point mutation at position chr3: 49412950 (human assembly GRCh37/hg19) where the wild-type allele is an adenine (A) and the mutant allele is a guanine (G). In a preferred embodiment, the set of reagents for detecting the RHOA F25L mutation comprises a mutation-specific probe of SEQ ID NO: 118, a wild-type probe of SEQ ID NO: 117, a forward primer of SEQ ID NO: 113 and a reverse primer of SEQ ID NO: 114. In another embodiment, a different forward and reverse primers can be chosen from ANY consecutive DNA sequence of size comprised between 15 and 25 base pairs selected in the following regions: chr3: 49412830-49412949 for the forward primer and chr3: 49412951-49413071 for the reverse primer (human assembly GRCh37/hg19), with the fundamental prerequisite that the position chr3: 49412950 must be excluded from the selected primer sequence. A different mutant and wild-type probe can be chosen from ANY consecutive DNA sequence of size comprised between 13 and 25 base pairs selected in the following regions: chr3: 49412925-49412975 (human assembly GRCh37/hg19) with the fundamental prerequisite that the position chr3: 49412950 must be included in the selected probe sequence.

Selected Primers for the Probe-Based F25L Assay

The following is an example of primers selected for the F25L assay:

TABLE 35
Table 35: Evaluation of all thermo-dynamic parameters considered to
select appropriate primers for the RHOA F₂₅L probe-assay.
Accepted values are presented with white background.

| | | | Tm | | GC % | self- | | Hetero- | Validated |
|---|---|---|---|---|---|---|---|---|---|
| Primer name | Sequence | Length | (° C.) | GC % | tail | dimers | Hairpins | dimers | (Y/N) |
| RHOA F25L L | GCACATACACCTCTGG | 16 | 55.8 | 56.2 | 71.4 | none | 24.8 | −3.16 | Y |
| RHOA F25 R | TGATTGTTGGTGATGG | 17 | 55.6 | 41.2 | 42.9 | none | none | | Y |
| | A | | | | | | | | |

| | | | | | | | | |
|---|---|---|---|---|---|---|---|---|
| Accepted values for the | 15-25 bp | 55-60° C. | 40-60% | >25% | > −4 | <Tm | > −4 | Y |
| parameters in analysis | | ΔTm r < 5° C. | | | Kcal/mol | | Kcal/mol | |

Selected Probes for the Probe-Based F25L Assay

The following is an example of probes selected for the F25L assay:

TABLE 36
Table 36: Evaluation of all thermodynamic parameters considered to
select appropriate probes for the RHOA F₂₅L probe-assay.
Accespted values are presented with white background.

| | | | | | | | | | | | Val- |
|---|---|---|---|---|---|---|---|---|---|---|---|
| | | | # | | | GC % | self- | Hair- | | Quen- | idated |
| Probe name | Sequence | Length | LNA | Tm (° C.) | GC % | tail | dimers | pins | Dyes | cher | (Y/N) |
| RHOA F25L | /5HEX/CTGA + | 20 | 1 | 61.8 | 45 | 57.1 | none | 10.9 | HEX | BHQ-1 | Y |
| WT | AGACTATGAGCAAGCA/ | | | | | | | | | | |
| | 3BHQ_1/ | | | | | | | | | | |
| RHOA F25L | /56-FAM/CTGA + | 19 | 1 | 61.9 | 52.6 | 57.1 | none | 10.9 | 6FAM | BHQ-1 | Y |
| Mut | GGACTATGAGCAAGC/ | | | | | | | | | | |
| | 3BHQ_1/ | | | | | | | | | | |

| | | | | | | | | | | |
|---|---|---|---|---|---|---|---|---|---|---|
| Accepted values for the | 13-25 bp | <4 | 55-60° C. | 40-60% | >25% | > −4 | «Tm | FAM: | BHQ-1 | Y |
| parameters in analysis | | | ΔTmr < 5° C. | | | Kcal/ | | mut | | |
| | | | | | | mol | | HEX: | | |
| | | | | | | | | WT | | |

Any other combination could be evaluated to fit the thermodynamic parameters presented above.

BLAT Alignment of the Validated Primer Pairs

>RHOA_F25L_L + RHOAF25L_R_81 bp
SEQ ID No: 179
GCACATACACCTCTGGgaactggtccttgctgaagactatgagcaagca
tgtctttccacaggcTCCATCACCAACAATCA

The proposed primers hit a secondary region of homology on chromosome 6. Nevertheless, as indicated from the characteristics of the reported homology (FIG. 17), the homology region does not cover the landing site of the forward primer, so that no amplification outside the true target region will be possible (FIG. 18). These primer combinations can hence be accepted and analysed further.

Thermodynamic Analysis

TABLE 37
Evaluation of heterodimers presence for all primers and
probes evaluated for the probe-based RHOA F25L assay.
Accepted values are presented with white background.

| | ΔG | Validated? |
|---|---|---|
| Oligonucleotide combination | (Kcal/mol) | Y/N |
| F25L L + F25L R | −3.16 | Y |
| F25L L + F25L mut | −3.03 | Y |
| F25L L + F25L WT | −1.18 | Y |
| F25L R + F25L mut | −0.69 | Y |
| F25L R + F25L WT | −0.69 | Y |
| Accepted values for the | >−4 Kcal/mol | Y |
| parameters in analysis | | |

RHOA G17V

The inventors have designed specific sets of primers and fluorescent probes allowing improved detection of the presence (or absence, or frequency) of such a mutation from a sample containing cfNA or genomic DNA.

More specifically, in a preferred embodiment, the mutation is RHOA G17V, where the glucine to valine change is due to a single point mutation at position chr3: 49412973 (human assembly GRCh37/hg19) where the wild-type allele is a cytosine (C) and the mutant allele is an adenine (A). In a preferred embodiment, the set of reagents for detecting the RHOA G17V mutation comprises a mutation-specific probe of SEQ ID NO: 120, a wild-type probe of SEQ ID NO: 119, a forward primer of SEQ ID NO: 115 and a reverse primer of SEQ ID NO: 116. In another embodiment, a different forward and reverse primers can be chosen from any consecutive DNA sequence of size comprised between 15 and 25 base pairs selected in the following regions: chr3: 49412853-49412972 for the forward primer and chr3: 49412974-49413093 for the reverse primer (human assembly GRCh37/hg19), with the fundamental prerequisite that the position chr3: 49412973 must be excluded from the selected primer sequence. A different mutant and wild-type probe can be chosen from ANY consecutive DNA sequence of size comprised between 13 and 25 base pairs selected in the following regions: chr3: 49412948-49412998 (human assembly GRCh37/hg19) with the fundamental prerequisite that the position chr3: 49412973 must be included in the selected probe sequence.

Selected Primers for the Probe-Based G17V Assay

The following is an example of primers selected for the G17V assay:

TABLE 38
Table 38: Evaluation of all thermodynamic parameters considered to select appropriate
primers for the RHOA G₁₇V probe-based assay. Accepted values are presented
with white background. Borderline values are presented in light grey background.

| Primer | | | | | GC % | self- | | Hetero- | Validated |
|---|---|---|---|---|---|---|---|---|---|
| name | Sequence | Length | Tm (° C.) | GC % | tail | dimers | Hairpins | dimers | (Y/N) |
| RHOA F | CCTTGCTGAA | 19 | 55.9 | 44.4 | 28.6 | −0.33 | 38.6 | −1.88 | Y |
| | GACTATGA | | | | | | | | |
| RHOA R | GTGCATTGCA | 20 | 56.3 | 42.1 | 14.3 | −3.8 | 34.5 | | Y |
| | GGTAATATC | | | | | | | | |

| | | | | | | | | |
|---|---|---|---|---|---|---|---|---|
| Accepted values | 15-25 bp | 55-60° C. | 40-60% | >25% | > −4 | <Tm | > −4 | Y |

| | | | |
|---|---|---|---|
| for the parameters | ΔTm r < 5° C. | Kcal/mol | Kcal/mol |
| in analysis | | | |

Selected Probes for the Probe-Based G17V Assay

The following is an example of probes selected for the G17V assay:

TABLE 39
Table 39: Evaluation of all thermodynamic parameters considered to select
appropriate probes for the RHOA G₁₇V probe-based assay.
Accepted values are presented with white background.

| | | | | | | | | | | | Val- |
|---|---|---|---|---|---|---|---|---|---|---|---|
| Probe | | | # | | | GC % | self- | Hair- | | | dated |
| name | Sequence | Length | LNA | Tm (° C.) | GC % | tails | dimers | pins | Dyes | Quencher | (Y/N) |
| RHOA | /5HEX/CATGTCTTT + | 19 | 1 | 62.8 | 52.6 | 71.4 | −2.17 | 24.3 | HEX | BHQ-1 | Y |
| G17V WT | CCACAGGCTC/3BHQ1/ | | | | | | | | | | |
| RHOA | /56-FAM/CATGTCTTT + | 19 | 1 | 58.9 | 47.4 | 71.4 | −2.17 | 35.8 | 6FAM | BHQ-1 | Y |
| G17V Mut | ACACAGGCTC/3BHQ1/ | | | | | | | | | | |

| | | | | | | | | | | |
|---|---|---|---|---|---|---|---|---|---|---|
| Accepted values for the | 13-25 bp | <4 | 55-60° C. | 40-60% | >25% | > −4 | «Tm | FAM: | BHQ-1 | Y |
| parameters in analysis | | | ΔTmr < | | | Kcal/ | | mut | | |
| | | | 5° C. | | | mol | | HEX: | | |
| | | | | | | | | WT | | |

Any other combination could be evaluated to fit the thermodynamic parameters presented above.

BLAT alignment of the validated primer pairs

>RHOA_L + RHOA_R_120 bp
SEQ ID No: 180
CCTTGCTGAAGACTATGAgcaagcatgtctttccacaggctccatcacc
aacaatcaccagtttcttccggatggcagccattgctgaaacacaaaac
acaGATATTACCTGCAATGCAC

The proposed primers hit a secondary region of homology on chromosome 6 and on chromosome 8. Nevertheless, as indicated from the characteristics of the reported homology (FIG. 19), the homology region does not cover the landing site of the reverse primer, so that no amplification outside the true target region will be possible. These primer combinations can hence be accepted and analysed further.

Thermodynamic Analysis

TABLE 40
Evaluation of heterodimers presence for all primers and
probes evaluated for the probe-based RHOA G17V assay.
Accepted values are presented with white background.

| | ΔG | Validated? |
|---|---|---|
| Oligonucleotide combination | (Kcal/mol) | Y/N |
| RHOA L + RHOA R | −1.88 | Y |
| RHOA L + G17V mut | −3.25 | Y |
| RHOA L + G17V WT | −3.25 | Y |
| RHOA R + G17V mut | −1.04 | Y |
| RHOA R + G17V WT | −2.17 | Y |
| Accepted values for the | >−4 Kcal/mol | Y |
| parameters in analysis | | |

RHOA C16STOP

The inventors have designed specific sets of primers allowing improved detection of the presence (or absence, or frequency) of such a mutation from a sample containing cfNA or genomic DNA.

More specifically, in a preferred embodiment, the mutation is RHOA C16*, where the cysteine to stop codon change is due to a non-sense single point mutation at position chr3: 49412975 (human assembly GRCh37/hg19) where the wild-type allele is an adenine (A) and the mutant allele is a thymidine (T). In a preferred embodiment, the set of reagents for detecting the RHOA C16* mutation comprises a mutation-specific probe of SEQ ID NO: 122, a wild-type probe of SEQ ID NO: 121, a forward primer of SEQ ID NO: 115 and a reverse primer of SEQ ID NO: 116. In another embodiment, a different forward and reverse primers can be chosen from ANY consecutive DNA sequence of size comprised between 15 and 25 base pairs selected in the following regions: chr3: 49412855-49412974 for the forward primer and chr3: 49412976-49413095 for the reverse primer (human assembly GRCh37/hg19), with the fundamental prerequisite that the position chr3: 49412975 must be excluded from the selected primer sequence. A different mutant and wild-type probe can be chosen from ANY consecutive DNA sequence of size comprised between 13 and 25 base pairs selected in the following regions: chr3: 49412950-49413000 (human assembly GRCh37/hg19) with the fundamental prerequisite that the position chr3: 49412975 must be included in the selected probe sequence.

Selected primers for the probe-based C16* assay

Given the proximity with the G17V position, the same primers (RHOA L and RHOA R) will be used for the C16* assay.

Selected probes for the probe-based C16* assay

The following is an example of probes selected for the C16* assay:

TABLE 41
Table 41: Evaluation of all thermodynamic parameters considered to select appropriate
probes for the RHOA C16* probe-based assay. Accepted values are presented with
white background.

| Probe | | | # | Tm | | GC % | self- | Hair- | | | Validated |
|---|---|---|---|---|---|---|---|---|---|---|---|
| name | Sequence | Length | LNA | (° C.) | GC % | tail | dimers | pins | Dyes | Quencher | (Y/N) |
| RHOA | /5HEX/TGTCTTTC | 20 | 1 | 64.5 | 50 | 57.1 | −0.08 | 18.3 | HEX | BHQ-1 | Y |
| C16* | C + ACAGGCTCCA | | | | | | | | | | |
| WT | T/3BHQ1/ | | | | | | | | | | |
| RHOA | /56-FAM/TGTCTT | 20 | 1 | 64.5 | 50 | 57.1 | −1.46 | 16.4 | 6FAM | BHQ-1 | Y |
| C16* | TCC + TCAGGCTC | | | | | | | | | | |
| Mut | CAT/3BHQ1/ | | | | | | | | | | |

| | | | | | | | | | | |
|---|---|---|---|---|---|---|---|---|---|---|
| Accepted values for | 13-25 bp | <4 | 55-60° C. | 40-60% | >25% | > −4 | «Tm | FAM: | BHQ-1 | Y |
| the parameters in | | | ΔTmr < | | | Kcal/mol | | mut | | |
| analysis | | | 5° C. | | | | | HEX: | | |
| | | | | | | | | WT | | |

Any other combination could be evaluated to fit the thermodynamic parameters as described above.

BLAT Alignment of the Validated Primer Pairs

Refer to correspondent BLAT alignment and analysis performed for the probe-based G17V assay.

Thermodynamic Analysis

TABLE 41
Evaluation of heterodimers presence for all primers and
probes evaluated for the probe-based RHOA C16* assay.
Accepted values are presented with white background.

| | ΔG | Validated? |
|---|---|---|
| Oligonucleotide combination | (Kcal/mol) | Y/N |
| RHOA L + RHOA R | −1.88 | Y |
| RHOA L + C16* mut | −3.25 | Y |
| RHOA L + C16* WT | −3.25 | Y |
| RHOA R + C16* mut | −1.46 | Y |
| RHOA R + C16* WT | −0.08 | Y |
| Accepted values for the | >−4 Kcal/ mol | Y |
| parameters in analysis | | |

RHOA G14V

The inventors have designed specific sets of primers allowing improved detection of the presence (or absence, or frequency) of such a mutation from a sample containing cfNA or genomic DNA.

More specifically, in a preferred embodiment, the mutation is RHOA G14V, where the glycine to valine change is due to a single point mutation at position chr3: 49412982 (human assembly GRCh37/hg19) where the wild-type allele is a cytosine (C) and the mutant allele is an adenine (A). In a preferred embodiment, the set of reagents for detecting the RHOA G14V mutation comprises a mutation-specific probe of SEQ ID NO: 124, a wild-type probe of SEQ ID NO: 123, a forward primer of SEQ ID NO: 115 and a reverse primer of SEQ ID NO: 116. In another embodiment, a different forward and reverse primers can be chosen from any consecutive DNA sequence of size comprised between 15 and 25 base pairs selected in the following regions: chr3: 49412862-49412981 for the forward primer and chr3: 49412983-49413102 for the reverse primer (human assembly GRCh37/hg19), with the fundamental prerequisite that the position chr3: 49412982 must be excluded from the selected primer sequence. A different mutant and wild-type probe can be chosen from any consecutive DNA sequence of size comprised between 13 and 25 base pairs selected in the following regions: chr3: 49412957-49413007 (human assembly GRCh37/hg19) with the prerequisite that the position chr3: 49412982 is included in the selected probe sequence.

Selected Primers for the Probe-Based G14V Assay

Given the proximity with the G17V position, the same primers (RHOA L and RHOA R) will be used for the G14V assay.

Selected Probes for the Probe-Based G14V Assay

The following is an example of probes selected for the G14V assay:

TABLE 42
Table 42: Evaluation of all thermodynamic parameters considered to select appropriate
probes for the RHOA G14V probe-based assay. Accepted values are presented with
white background.

| Probe | | | # | Tm | | GC % | self- | Hair- | | | Validated |
|---|---|---|---|---|---|---|---|---|---|---|---|
| name | Sequence | Length | LNA | (° C.) | GC % | tail | dimers | pins | Dyes | Quencher | (Y/N) |
| RHOA | /5HEX/CACAGGCT + | 19 | 1 | 65.2 | 57.9 | 57.1 | none | 31.1 | HEX | BHQ-1 | Y |
| G14V | CCATCACCAAC/3BHQ_1/ | | | | | | | | | | |
| WT | | | | | | | | | | | |
| RHOA | /56-FAM/CACAGGCT + | 19 | 1 | 61.3 | 52.6 | 57.1 | none | 31.1 | 6FAM | BHQ-1 | Y |
| G14V | ACATCACCAAC/3BHQ_1/ | | | | | | | | | | |

Mut

| | | | | | | | | | | |
|---|---|---|---|---|---|---|---|---|---|---|
| Accepted values for | 13-25 bp | <4 | 55-60° C. | 40-60% | >25% | > −4 | «Tm | FAM: | BHQ-1 | Y |
| the parameters in | | | ΔTmr < | | | Kcal/ | | mut | | |
| analysis | | | 5° C. | | | mol | | HEX: | | |
| | | | | | | | | WT | | |

Any other combination could be evaluated to fit the thermodynamic parameters presented above.

BLAT Alignment of the Validated Primer Pairs

Refer to correspondent BLAT alignment and analysis performed for the probe-based G17V assay.

Thermodynamic Analysis

TABLE 43
Evaluation of heterodimers presence for all primers and
probes evaluated for the probe-based RHOA G14V assay.
Accepted values are presented with white background.

| | ΔG | Validated? |
|---|---|---|
| Oligonucleotide combination | (Kcal/mol) | Y/N |
| RHOA L + RHOA R | −1.88 | Y |
| RHOA L + G14V mut | −1.46 | Y |
| RHOA L + G14V WT | −1.46 | Y |
| RHOA R + G14V mut | −1.2 | Y |
| RHOA R + G14V WT | −1.2 | Y |
| Accepted values for the | >−4 Kcal/ mol | Y |
| parameters in analysis | | |

SEQUENCES

| SEQ ID | | | |
|---|---|---|---|
| number | Name | Sequence | Mutation |
| 1 | F₂₅L₂₂ | CTGGGAACTGGTCCTTGCTGAG | F25L |
| 2 | F₂₅L₂₁ | TGGGAACTGGTCCTTGCTGAG | |
| 3 | F₂₅L₂₀ | GGGAACTGGTCCTTGCTGAG | |
| 4 | F₂₅L₁₉ | GGAACTGGTCCTTGCTGAG | |
| 5 | F₂₅L₁₈ | GAACTGGTCCTTGCTGAG | |
| 6 | F₂₅L₁₇ | AACTGGTCCTTGCTGAG | |
| 7 | F₂₅L₁₆ | ACTGGTCCTTGCTGAG | |
| 8 | F₂₅L I₂₂ | AAGACATGCTTGCTCATAGTCC | |
| 9 | F₂₅L I₂₁ | AGACATGCTTGCTCATAGTCC | |
| 10 | F₂₅L I₂₀ | GACATGCTTGCTCATAGTCC | |
| 11 | F₂₅L I₁₉ | ACATGCTTGCTCATAGTCC | |
| 12 | F₂₅L I₁₈ | CATGCTTGCTCATAGTCC | |
| 13 | F₂₅L I₁₇ | ATGCTTGCTCATAGTCC | |
| 14 | F₂₅L I₁₆ | TGCTTGCTCATAGTCC | |
| 15 | F₂₅ P₂₀ | AGAAACTGGTGATTGTTGGT | |
| 16 | F₂₅ I P₂₀ P₃ | CCTCGATATCTGCCACATAG | |
| 17 | F₂₅ I P₂₁ | CCACATAGTTCTCAAACACTG | |
| 18 | F₂₅ I P₂₀ | CACATAGTTCTCAAACACTG | |
| 19 | F₂₅ I P₁₉ | ACATAGTTCTCAAACACTG | |
| 20 | F₂₅ I P₁₈ | CATAGTTCTCAAACACTG | |
| 21 | F₂₅L OB₂₃ | TGGTCCTTGCTGAAGACTATGAG | |
| 22 | F₂₅L OB₂₂ | GGTCCTTGCTGAAGACTATGAG | |
| 23 | F₂₅L OB₂₁ | GTCCTTGCTGAAGACTATGAG | |
| 24 | F₂₅L OB₂₀ | TCCTTGCTGAAGACTATGAG | |
| 25 | F₂₅L OB₁₉ | CCTTGCTGAAGACTATGAG | |
| 26 | F₂₅Li OB₂₃ | TGCTCATAGTCTTCAGCAAGGAC | |
| 27 | F₂₅Li OB₂₂ | GCTCATAGTCTTCAGCAAGGAC | |
| 28 | F₂₅Li OB₂₁ | GCTCATAGTCTTCAGCAAGGA | |
| 29 | F₂₅Li OB₂₁/₂ | CTCATAGTCTTCAGCAAGGAC | |
| 30 | F₂₅Li OB₂₀ | CTCATAGTCTTCAGCAAGGA | |
| 31 | F₂₅Li OB₁₉ | CTCATAGTCTTCAGCAAGG | |
| 32 | F₂₅ WT L | GCAGGATGAGAATGGATTC | |
| 33 | F₂₅ WT R | GAATTAGAGCTTTTTGCCTC | |
| 34 | F₂₅Li OB₂₃/₂ | ACATGCTTGCTCATAGTCTTCAG | |
| 35 | F₂₅Li OB₂₂/₂ | CATGCTTGCTCATAGTCTTCAG | |
| 36 | F₂₅Li OB₂₁/₃ | ATGCTTGCTCATAGTCTTCAG | |
| 37 | F₂₅Li OB₂₀/₂ | TGCTTGCTCATAGTCTTCAG | |
| 38 | F₂₅Li OB₁₉/₂ | GCTTGCTCATAGTCTTCAG | |
| 39 | G₁₇V₂₂ | ACTATGAGCAAGCATGTCTTTA | G17V |
| 40 | G₁₇V₂₁ | CTATGAGCAAGCATGTCTTTA | |
| 41 | G₁₇V₂₀ | TATGAGCAAGCATGTCTTTA | |
| 42 | G₁₇V₁₉ | ATGAGCAAGCATGTCTTTA | |
| 43 | G₁₇V₁₈ | TGAGCAAGCATGTCTTTA | |
| 44 | G₁₇V₁₇ | GAGCAAGCATGTCTTTA | |
| 45 | G₁₇V₁₆ | AGCAAGCATGTCTTTA | |
| 46 | G₁₇V I₂₂ | TTGTTGGTGATGGAGCCTGTGT | |
| 47 | G₁₇V I₂₁ | TGTTGGTGATGGAGCCTGTGT | |
| 48 | G₁₇V I₂₀ | GTTGGTGATGGAGCCTGTGT | |
| 49 | G₁₇V I₁₉ | TTGGTGATGGAGCCTGTGT | |
| 50 | G₁₇V I₁₈ | TGGTGATGGAGCCTGTGT | |
| 51 | G₁₇V I₁₇ | GGTGATGGAGCCTGTGT | |
| 52 | G₁₇V I₁₆ | GTGATGGAGCCTGTGT | |
| 53 | G₁₇ I P₁₈ | TTCTCAAACACTGTGGGC | |
| 54 | G₁₇ I P₁₉ | GAACTGGTCCTTGCTGAAG | |
| 55 | G₁₇ I P₂₀ | TCTGCCACATAGTTCTCAAA | |
| 56 | G₁₇ P₂₁ | TCTGTGTTTTGTGTTTCAGCA | |
| 57 | G₁₇ P₂₀ | CTGTGTTTTGTGTTTCAGCA | |
| 58 | G₁₇ P₁₉ | TGTGTTTTGTGTTTCAGCA | |
| 59 | G₁₇ P₁₈ | GTGTTTTGTGTTTCAGCA | |
| 60 | G₁₇ OB₂₃ | CATGTCTTTCCACAGGCTCCATC | |
| 61 | G₁₇ OB₂₂ | ATGTCTTTCCACAGGCTCCATC | |
| 62 | G₁₇ OB₂₁ | ATGTCTTTCCACAGGCTCCAT | |
| 63 | G₁₇ OB₂₀ | ATGTCTTTCCACAGGCTCCA | |
| 64 | G₁₇ OB₁₉ | ATGTCTTTCCACAGGCTCC | |
| 65 | G₁₇ I OB₂₃ | ATGGAGCCTGTGGAAAGACATGC | |
| 66 | G₁₇ I OB₂₂ | ATGGAGCCTGTGGAAAGACATG | |
| 67 | G₁₇ I OB₂₁ | ATGGAGCCTGTGGAAAGACAT | |
| 68 | G₁₇ I OB₂₀ | ATGGAGCCTGTGGAAAGACA | |
| 69 | G₁₇ I OB₁₉ | TGGAGCCTGTGGAAAGACA | |
| 70 | G₁₇ WT L | GACACATGCTTTGGTAACC | |
| 71 | G₁₇ WT R | CTTTTGGTGCCAGGTGGA | |
| 72 | C₁₆*₂₂ | TATGAGCAAGCATGTCTTTCCT | C16Stop |
| 73 | C₁₆*₂₁ | ATGAGCAAGCATGTCTTTCCT | |
| 74 | C₁₆*₂₀ | TGAGCAAGCATGTCTTTCCT | |
| 75 | C₁₆*₁₉ | GAGCAAGCATGTCTTTCCT | |
| 76 | C₁₆*₁₈ | AGCAAGCATGTCTTTCCT | |
| 77 | C₁₆*₁₇ | GCAAGCATGTCTTTCCT | |
| 78 | C₁₆*₁₆ | CAAGCATGTCTTTCCT | |
| 79 | C₁₆* I₂₂ | GATTGTTGGTGATGGAGCCTGA | |
| 80 | C₁₆* I₂₁ | ATTGTTGGTGATGGAGCCTGA | |
| 81 | C₁₆* I₂₀ | TTGTTGGTGATGGAGCCTGA | |
| 82 | C₁₆* I₁₉ | TGTTGGTGATGGAGCCTGA | |
| 83 | C₁₆* I₁₈ | GTTGGTGATGGAGCCTGA | |
| 84 | C₁₆* I₁₇ | TTGGTGATGGAGCCTGA | |
| 85 | C₁₆* I₁₆ | TGGTGATGGAGCCTGA | |
| 86 | C₁₆* P₂₀ | GTGTTTTGTGTTTCAGCAAT | |
| 87 | C₁₆* P₂₁ | GTGTTTTGTGTTTCAGCAATG | |
| 88 | C₁₆* I P₂₀ | ATAGTTCTCAAACACTGTGG | |
| 89 | G₁₄V₂₂ | AAGCATGTCTTTCCTCAGGCTA | G14V |
| 90 | G₁₄V₂₁ | AGCATGTCTTTCCTCAGGCTA | |
| 91 | G₁₄V₂₀ | GCATGTCTTTCCTCAGGCTA | |
| 92 | G₁₄V₁₉ | CATGTCTTTCCTCAGGCTA | |
| 93 | G₁₄V₁₈ | ATGTCTTTCCTCAGGCTA | |
| 94 | G₁₄V₁₇ | TGTCTTTCCTCAGGCTA | |
| 95 | G₁₄V₁₆ | GTCTTTCCTCAGGCTA | |
| 96 | G₁₄V I₂₂ | AACTGGTGATTGTTGGTGATGT | |
| 97 | G₁₄V I₂₁ | ACTGGTGATTGTTGGTGATGT | |
| 98 | G₁₄V I₂₀ | CTGGTGATTGTTGGTGATGT | |
| 99 | G₁₄V I₁₉ | TGGTGATTGTTGGTGATGT | |
| 100 | G₁₄V I₁₈ | GGTGATTGTTGGTGATGT | |
| 101 | G₁₄V I₁₇ | GTGATTGTTGGTGATGT | |
| 102 | G₁₄V I₁₆ | TGATTGTTGGTGATGT | |
| 103 | G₁₄ OB₂₃ | TTCCACAGGCTCCATCACCAACA | |
| 104 | G₁₄ OB₂₂ | TTCCACAGGCTCCATCACCAAC | |
| 105 | G₁₄ OB₂₁ | TCCACAGGCTCCATCACCAAC | |
| 106 | G₁₄ OB₂₀ | TCCACAGGCTCCATCACCAA | |
| 107 | G₁₄ OB₁₉ | CCACAGGCTCCATCACCAA | |
| 108 | G₁₄ I OB₂₃ | TGTTGGTGATGGAGCCTGTGGAA | |
| 109 | G₁₄ I OB₂₂ | TGTTGGTGATGGAGCCTGTGGA | |
| 110 | G₁₄ I OB₂₁ | GTTGGTGATGGAGCCTGTGGA | |
| 111 | G₁₄ I OB₂₀ | TTGGTGATGGAGCCTGTGGA | |
| 112 | G₁₄ I OB₁₉ | TTGGTGATGGAGCCTGTGG | |

SEQUENCES FOR PROBE-BASED ASSAYS
Probe-based assays

| SEQ ID number | Name | Sequence | Mutation |
|---|---|---|---|
| 113 | RHOA F25L L | GCACATACACCTCTGG | F25L |
| 114 | RHOA F25 R | TGATTGTTGGTGATGGA | |
| 115 | RHOA F | CCTTGCTGAAGACTATGA | G17V, C16*, |
| 116 | RHOA R | GTGCATTGCAGGTAATATC | G14V |
| 117 | RHOA F25L WT | /5HEX/CTGA + AGACTATGAGCAAGCA/3BHQ_1/ | F25L |
| 118 | RHOA F25L Mut | /56-FAM/CTGA + GGACTATGAGCAAGC/3BHQ_1/ | |
| 119 | RHOA G17V WT | /5HEX/CATGTCTTT + CCACAGGCTC/3BHQ_1/ | G17V |
| 120 | RHOA G17V Mut | /56-FAM/CATGTCTTT + ACACAGGCTC/3BHQ_1/ | |
| 121 | RHOA C16* WT | /5HEX/TGTCTTTCC + ACAGGCTCCAT/3BHQ_1/ | C16* |
| 122 | RHOA C16* Mut | /56-FAM/TGTCTTTCC + TCAGGCTCCAT/3BHQ_1/ | |
| 123 | RHOA G14V WT | /5HEX/CACAGGCT + CCATCACCAAC/3BHQ_1/ | G14V |
| 124 | RHOA G14V Mut | /56-FAM/CACAGGCT + ACATCACCAAC/3BH Q_1/ | |

1. A polymerase chain reaction (PCR) method for determining the presence or absence of a mutation in the Ras homologue gene family member A (RHOA) gene in a sample obtained from a subject, the method comprising:
i) forming a mixture comprising the sample and a primer set, wherein the sample comprises a RHOA encoding nucleotide target sequence and the primer set comprises;
(a) a mutation-specific forward primer;
(b) a reverse primer; and
(c) an oligoblocker,
ii) subjecting the mixture of step (i) to, sequentially in steps, a denaturing step, an annealing step, and an extension step, wherein in the annealing step the forward primer and the oligoblocker compete to anneal to the target sequence, wherein the oligoblocker is capable of specifically hybridizing to a wild-type target sequence to block polymerase extension and the mutation-specific forward primer is capable of hybridizing to a mutated RHOA nucleotide sequence; and
iii) determining whether a PCR product is obtained through steps (i) and (ii), wherein the presence of the PCR product is indicative of the presence of a mutation in the RHOA gene in the sample, and the absence of the PCR product is indicative of the absence of a mutation in the RHOA gene in the sample.

2. The method according to claim 1, wherein the sample is selected from a biological fluid sample, mononucleated white cells, polymorphonuclear leukocytes or tumour tissue, and/or wherein the sample is a biological fluid sample which comprises cell free nucleic acids (cfNA), and/or wherein the mutation in the RHOA gene results in an amino acid substitution, optionally wherein the substitution is selected from the group consisting of G14V, C16stop, G17V and F25L, and/or wherein the mutation-specific forward primer is capable of hybridizing to the nucleotide sequence as substantially as set out in SEQ ID NO: 127, 128, 129, 130, 131, 132, 133 or 134, or a fragment or variant thereof.

3.-5. (canceled)

6. The method according to claim 1, wherein the mutation-specific forward primer sequence is a nucleotide sequence of any one of SEQ ID NO: 1-14 or a variant or fragment thereof, preferably any one of SEQ ID NO: 5-7, 11-16 or a variant or fragment thereof, and is capable of hybridizing to a mutated RHOA nucleotide sequence resulting in the amino acid substitution F25L, and/or wherein the mutation-specific forward primer sequence is be a nucleotide sequence of any one of SEQ ID NO: 39-52 or a variant or fragment thereof, preferably any one of SEQ ID NO: 43-44, 50-51 or a variant or fragment thereof, and is capable of hybridizing to a mutated RHOA nucleotide sequence comprising a mutation resulting in the amino acid substitution G17V.

7. (canceled)

8. The method according to claim 1, wherein the mutation-specific forward primer sequence is a nucleotide sequence of any one of SEQ ID NO: 72-85 or a variant or fragment thereof, preferably any one of SEQ ID NO: 75-78, 83-85 or a variant or fragment thereof, and is capable of hybridizing to a mutated RHOA nucleotide sequence resulting in the amino acid substitution C16stop, and/or wherein the mutation-specific forward primer sequence is a nucleotide sequence of any one of SEQ ID NO: 89 — 102 or a variant or fragment thereof, preferably any one of SEQ ID NO: 92-95, 99-101 or a variant or fragment thereof, and is capable of hybridizing to a mutated RHOA nucleotide sequence resulting in the amino acid substitution G14V.

9. (canceled)

10. The method according to claim 1, wherein:
i) the mutated RHOA nucleotide sequence results in the amino acid substitution F25L, and the mutation-specific forward primer is selected from any one of SEQ ID NO: 1-14, the reverse primer is selected from any one of SEQ ID NO: 15-20, and the oligoblocker is selected from any one of SEQ ID NO: 21-31, 34-38;
ii) the mutated RHOA nucleotide sequence results in the amino acid substitution G17V and the mutation-specific forward primer is selected from anyone of any one of SEQ ID NOs: 39-52, the reverse primer is selected from any one of SEQ ID NO: 53-59, and the oligoblocker is selected from any one of SEQ ID NO: 60-69; and/or
iii) the mutated RHOA nucleotide sequence results in the amino acid substitution RHOA C16Stop and wherein the mutation-specific primer is selected from any one of SEQ ID NO: 72-85, the reverse primer is selected from any one of SEQ ID NO: 53-59, 86-88, and the oligoblocker is selected from any one of SEQ ID NO: 60-69; and/or
iv) the mutated RHOA nucleotide sequence results in the amino acid substitution RHOA G14V and the mutation-specific primer is selected from any one of SEQ ID NOs: 89-102, the reverse primer is selected from any one of SEQ ID NO: 53-59, 86-88, and the oligoblocker is selected from SEQ ID NO: 60-69, 103-112.

11. A polymerase chain reaction (PCR) method for determining the presence or absence of a mutation in the Ras homologue gene family member A (RHOA) gene in a sample obtained from a subject, the method comprising:
i) forming a mixture comprising the sample, a wild-type forward primer, a wild-type reverse primer, and a mutation specific fluorescent probe, wherein the sample comprises a RHOA encoding nucleotide target sequence;
ii) subjecting the mixture of step (i) to, sequentially in steps, a denaturing step, an annealing step, and an extension step, wherein the mutant specific fluorescent probe is capable of hybridizing to a mutated RHOA nucleotide sequence and the wild-type forward primer and wild-type reverse primer are capable of hybridizing to a wild-type RHOA encoding nucleotide target sequence; and
iii) detecting a fluorescent signal obtained by the reaction of step ii), wherein the presence of a fluorescent signal in a channel associated with the mutant specific fluorescent probe is indicative of the presence of a mutation in the RHOA gene, and the absence of a fluorescent signal is indicative of the absence of a mutation in the RHOA gene.

12. The method according to claim 11, wherein the wild-type forward primer and wild-type reverse primer are capable of hybridizing to a different region of the RHOA nucleotide sequence than the mutation-specific forward primer and reverse primer are capable of hybridizing to, and/or wherein:
i) the mutated RHOA nucleotide sequence results in the amino acid substitution RHOA F25L, and the mutation specific fluorescent probe comprises a nucleic acid sequence substantially as set out in SEQ ID NO: 118, or a fragment or variant thereof;
ii) wherein the mutated RHOA nucleotide sequence results in the amino acid substitution G17V, and the mutation specific fluorescent probe comprises a nucleic acid sequence substantially as set out in SEQ ID NO: 120, or a fragment or variant thereof;
iii) wherein the mutated RHOA nucleotide sequence results in the amino acid substitution C16Stop, and the mutation specific fluorescent probe comprises a nucleic acid sequence substantially as set out in SEQ ID NO: 122, or a fragment or variant thereof; and/or
iv) wherein the mutated RHOA nucleotide sequence results in the amino acid substitution G14V, and the mutation specific fluorescent probe comprises a nucleic acid sequence substantially as set out in SEQ ID NO: 124, or a fragment or variant thereof.

13. (canceled)

14. The method according to claim 11, wherein the method further comprises adding to the mixture of step i) a wild-type fluorescent probe that is substantially complementary to the wild-type RHOA nucleotide sequence, and is labelled with a different fluorophore with emission spectrum non-overlapping with the mutation specific fluorescent probe, optionally wherein:
i) the mutated RHOA nucleotide sequence results in the amino acid substitution F25L, and the wild-type florescent probe comprises a nucleic acid sequence substantially as set out in SEQ ID NO: 117, or a fragment or variant thereof;
ii) wherein the mutated RHOA nucleotide sequence results in the amino acid substitution G17V, the wild-type florescent probe comprises a nucleic acid sequence substantially as set out in SEQ ID NO: 119, or a fragment or variant thereof;
iii) wherein the mutated RHOA nucleotide sequence results in the amino acid substitution C16Stop, the wild-type fluorescent probe comprises a nucleic acid sequence substantially as set out in SEQ ID NO: 121, or a fragment or variant thereof; and/or
iv) wherein the mutated RHOA nucleotide sequence results in the amino acid substitution G14V, the wild-type fluorescent probe comprises a nucleic acid sequence substantially as set out in SEQ ID NO: 123, or a fragment or variant thereof.

15. (canceled)

16. The method according to claim 11, wherein:
i) the mutated RHOA nucleotide sequence results in the amino acid substitution F25L, the wild-type forward primer comprises a nucleic acid sequence as substantially set out in SEQ ID NO: 113, or a fragment or variant thereof, and the wild-type reverse primer comprises a nucleic acid sequence as substantially set out in SEQ ID NO: 114, or a fragment or variant thereof; and/or
ii) wherein the mutated RHOA nucleotide sequence results in the amino acid substitution G17V, C16Stop or G14V the wild-type forward primer comprises a nucleic acid sequence as substantially set out in SEQ ID NO: 115, or a fragment or variant thereof, the wild-type reverse primer comprises a nucleic acid sequence as substantially set out in SEQ ID NO: 116, or a fragment or variant thereof.

17. A polymerase chain reaction (PCR) method for determining the frequency of RHOA gene mutations in a sample obtained from a subject, the method comprising:
i) forming a mixture comprising the sample and a first primer set, wherein the sample comprises RHOA encoding nucleotide first and second target sequences and the first primer set is the primer set according to claim 1;
ii) (a) contacting the mixture of step i) with a second primer set comprising a wild-type forward primer and a wild-type reverse primer; or
(b) forming a second mixture comprising the sample and a second primer set comprising a wild-type forward primer and a wild-type reverse primer;
iii) subjecting the mixture of step ii) (a) or the mixtures of step i) and ii) (b) to, sequentially in steps, a denaturing step, an annealing step, and an extension step, wherein in the annealing step the mutation-specific forward primer and the oligoblocker compete to anneal to the first target sequence, wherein the oligoblocker is capable of specifically hybridizing to a wild-type target sequence to block polymerase extension, and the forward primer anneals to the second target sequence, wherein the second target sequence corresponds to a wild-type sequence of the RHOA gene; and
iv) obtaining at least one PCR product through steps (i) to (iii), wherein the presence of a first product amplified for the first target sequence resulting from the first primer set is indicative of the presence of a mutation in the RHOA gene in the sample and the presence of a second product amplified product from the second target sequence resulting from the second primer set is indicative of the total number of RHOA encoding nucleotide sequences in the sample; and
v) comparing the amount of sequence amplified from the first and second target sequences to determine the frequency of RHOA gene mutations in a sample.

18. The method according to claim 17, wherein the wild-type forward primer and wild-type reverse primer are capable of hybridizing to a different region of the RHOA nucleotide sequence than the mutation-specific forward primer and reverse primer are capable of hybridizing to.

19. The method according to claim 17, wherein the first primer set is as defined in claim 2.

20. A method of diagnosing cancer in a subject, or a predisposition thereto, or for providing a prognosis of cancer, comprising:
a) i) performing a polymerase chain reaction (PCR) method according to claim 1; and
ii) detecting the presence of a PCR product obtained through step (i) to diagnose or prognose cancer in the subject, wherein the presence of a PCR product is indicative of cancer;
b) i) performing a polymerase chain reaction (PCR) method according to claim 11; and
ii) detecting the presence of a fluorescent signal obtained by step i) to diagnose or prognose cancer in the subject, wherein the presence of a fluorescent signal is indicative of cancer; or
c) i) performing a polymerase chain reaction (PCR) method according to claim 17; and
ii) comparing the amount of sequence amplified from the first and second target sequences to determine the frequency of RHOA gene mutations in a sample, thereby diagnosing or prognosing cancer in the subject.

21. The method according to claim 20, wherein the cancer is peripheral T-cell lymphoma.

22. A mutation-specific forward primer that is capable of hybridizing to the nucleotide sequence as substantially as set out in SEQ ID NO: 127, 128, 129, 130, 131, 132, 133 or 134, or a fragment or variant thereof, optionally wherein the mutation-specific primer sequence is a nucleotide sequence of any one of SEQ ID NO: 1-14, 39-52, 72-85 and 89-102 or a variant or fragment thereof.

23. (canceled)

24. A mutation-specific forward primer and reverse primer pair, wherein the mutation specific forward primer is capable of hybridizing to a mutated RHOA nucleotide sequence, wherein:
i) the mutated RHOA nucleotide sequence results in the amino acid substitution F25L, the mutation-specific forward primer is selected from any one of SEQ ID NO: 1-14 and the reverse primer is selected from any one of SEQ ID NO: 15-20;
ii) wherein the mutated RHOA nucleotide sequence results in the amino acid substitution G17V, the mutation-specific forward primer is selected from anyone of any one of SEQ ID NOs: 39-52 and the reverse primer is selected from any one of SEQ ID NO: 53-59;
iii) wherein the mutated RHOA nucleotide sequence results in the amino acid substitution RHOA C16Stop, the mutation-specific primer is selected from any one of SEQ ID NO: 72-85 and the reverse primer is selected from any one of SEQ ID NO: 53-59, 86-88; or
iv) wherein the mutated RHOA nucleotide sequence results in the amino acid substitution G14V, the mutation-specific primer is selected from any one of SEQ ID NOs: 89-102, the reverse primer is selected from any one of SEQ ID NO: 53-59, 86-88; or a primer set comprising a mutation-specific forward primer a reverse primer and an oligoblocker, wherein the mutation specific forward primer is capable of hybridizing to a mutated RHOA nucleotide sequence, wherein:
i) the mutated RHOA nucleotide sequence results in the amino acid substitution F25L, and the mutation-specific forward primer is selected from any one of SEQ ID NO: 1-14, the reverse primer is selected from any one of SEQ ID NO: 15-20, and the oligoblocker is selected from any one of SEQ ID NO: 21-31, 34-38;
ii) the mutated RHOA nucleotide sequence results in the amino acid substitution G17V and the mutation-specific forward primer is selected from anyone of any one of SEQ ID NOs: 39-52, the reverse primer is selected from any one of SEQ ID NO: 53-59, and the oligoblocker is selected from any one of SEQ ID NO: 60-69;
iii) the mutated RHOA nucleotide sequence results in the amino acid substitution RHOA C16Stop and wherein the mutation-specific primer is selected from any one of SEQ ID NO: 72-85, the reverse primer is selected from any one of SEQ ID NO: 53-59, 86-88, and the oligoblocker is selected from any one of SEQ ID NO: 60-69; or
iv) the mutated RHOA nucleotide sequence results in the amino acid substitution RHOA G14V and the mutation-specific primer is selected from any one of SEQ ID NOs: 89-102, the reverse primer is selected from any one of SEQ ID NO: 53-59, 86-88, and the oligoblocker is selected from SEQ ID NO: 60-69, 103-112.

25. (canceled)

26. A mutation specific fluorescent probe capable of hybridizing to a mutated RHOA nucleotide sequence, wherein:
i) the mutated RHOA nucleotide sequence results in the amino acid substitution RHOA F25L, and the mutation specific fluorescent probe comprises a nucleic acid sequence substantially as set out in SEQ ID NO: 118, or a fragment or variant thereof;
ii) wherein the mutated RHOA nucleotide sequence results in the amino acid substitution G17V, and the mutation specific fluorescent probe comprises a nucleic acid sequence substantially as set out in SEQ ID NO: 120, or a fragment or variant thereof;
iii) wherein the mutated RHOA nucleotide sequence results in the amino acid substitution C16Stop, and the mutation specific fluorescent probe comprises a nucleic acid sequence substantially as set out in SEQ ID NO: 122, or a fragment or variant thereof; or
iv) wherein the mutated RHOA nucleotide sequence results in the amino acid substitution G14V, and the mutation specific fluorescent probe comprises a nucleic acid sequence substantially as set out in SEQ ID NO: 124, or a fragment or variant thereof.

27. A mutation specific fluorescent probe and wild-type specific fluorescent probe pair, wherein the mutation specific fluorescent probe is as defined in claim 26 and the wild-type fluorescent probe is substantially complementary to a wild-type RHOA nucleotide sequence, and is labelled with a different fluorophore with emission spectrum non-overlapping with the mutation specific fluorescent probe.

28. (canceled)

29. A method of diagnosing cancer in a subject, comprising:
contacting a sample obtained from the subject with the mutation-specific forward primer of claim 22, the mutation-specific forward primer and reverse primer pair of claim 24, the primer set of claim 24, the mutation specific fluorescent probe of claim 26 or the mutation specific fluorescent probe and wild-type specific fluorescent probe pair of claim 27;
performing a polymerase chain reaction (PCR) method; and
detecting the presence of a PCR product, wherein the presence of a PCR product is indicative of cancer, or detecting the presence of a fluorescent signal, wherein the presence of a fluorescent signal is indicative of cancer, optionally wherein the cancer is peripheral T-cell lymphoma.

30. (canceled)

31. A kit for determining the presence, absence or frequency of a mutation in the RHOA gene, comprising:
a) i) at least one mutation-specific forward primer of claim 22, at least one mutation-specific forward primer and reverse primer pair of claim 24, at least one primer set of claim 24; or
ii) at least one mutation specific fluorescent probe of claim 26 or at least one mutation specific fluorescent probe and wild-type specific fluorescent probe pair of claim 27;
b) a DNA polymerase
c) optionally, a sample obtained from a subject; and
d) instructions for use.


SEQUENCE LISTING (189 sequences)

SEQ ID NO: 1 - DNA, 22 bp, Artificial Sequence
  Synthetic primer
ctgggaactggtccttgctgag

SEQ ID NO: 2 - DNA, 21 bp, Artificial Sequence
  Synthetic primer
tgggaactggtccttgctgag

SEQ ID NO: 3 - DNA, 20 bp, Artificial Sequence
  Synthetic primer
gggaactggtccttgctgag

SEQ ID NO: 4 - DNA, 19 bp, Artificial Sequence
  Synthetic primer
ggaactggtccttgctgag

SEQ ID NO: 5 - DNA, 18 bp, Artificial Sequence
  Synthetic primer
gaactggtccttgctgag

SEQ ID NO: 6 - DNA, 17 bp, Artificial Sequence
  Synthetic primer
aactggtccttgctgag

SEQ ID NO: 7 - DNA, 16 bp, Artificial Sequence
  Synthetic primer
actggtccttgctgag

SEQ ID NO: 8 - DNA, 22 bp, Artificial Sequence
  Synthetic primer
aagacatgcttgctcatagtcc

SEQ ID NO: 9 - DNA, 21 bp, Artificial Sequence
  Synthetic primer
agacatgcttgctcatagtcc

SEQ ID NO: 10 - DNA, 20 bp, Artificial Sequence
  Synthetic primer
gacatgcttgctcatagtcc

SEQ ID NO: 11 - DNA, 19 bp, Artificial Sequence
  Synthetic primer
acatgcttgctcatagtcc

SEQ ID NO: 12 - DNA, 18 bp, Artificial Sequence
  Synthetic primer
catgcttgctcatagtcc

SEQ ID NO: 13 - DNA, 17 bp, Artificial Sequence
  Synthetic primer
atgcttgctcatagtcc

SEQ ID NO: 14 - DNA, 16 bp, Artificial Sequence
  Synthetic primer
tgcttgctcatagtcc

SEQ ID NO: 15 - DNA, 20 bp, Artificial Sequence
  Synthetic primer
agaaactggtgattgttggt

SEQ ID NO: 16 - DNA, 20 bp, Artificial Sequence
  Synthetic primer
cctcgatatctgccacatag

SEQ ID NO: 17 - DNA, 21 bp, Artificial Sequence
  Synthetic primer
ccacatagttctcaaacactg

SEQ ID NO: 18 - DNA, 20 bp, Artificial Sequence
  Synthetic primer
cacatagttctcaaacactg

SEQ ID NO: 19 - DNA, 19 bp, Artificial Sequence
  Synthetic primer
acatagttctcaaacactg

SEQ ID NO: 20 - DNA, 18 bp, Artificial Sequence
  Synthetic primer
catagttctcaaacactg

SEQ ID NO: 21 - DNA, 23 bp, Artificial Sequence
  Synthetic primer
tggtccttgctgaagactatgag

SEQ ID NO: 22 - DNA, 22 bp, Artificial Sequence
  Synthetic primer
ggtccttgctgaagactatgag

SEQ ID NO: 23 - DNA, 21 bp, Artificial Sequence
  Synthetic primer
gtccttgctgaagactatgag

SEQ ID NO: 24 - DNA, 20 bp, Artificial Sequence
  Synthetic primer
tccttgctgaagactatgag

SEQ ID NO: 25 - DNA, 19 bp, Artificial Sequence
  Synthetic primer
ccttgctgaagactatgag

SEQ ID NO: 26 - DNA, 23 bp, Artificial Sequence
  Synthetic primer
tgctcatagtcttcagcaaggac

SEQ ID NO: 27 - DNA, 22 bp, Artificial Sequence
  Synthetic primer
gctcatagtcttcagcaaggac

SEQ ID NO: 28 - DNA, 21 bp, Artificial Sequence
  Synthetic primer
gctcatagtcttcagcaagga

SEQ ID NO: 29 - DNA, 21 bp, Artificial Sequence
  Synthetic primer
ctcatagtcttcagcaaggac

SEQ ID NO: 30 - DNA, 20 bp, Artificial Sequence
  Synthetic primer
ctcatagtcttcagcaagga

SEQ ID NO: 31 - DNA, 19 bp, Artificial Sequence
  Synthetic primer
ctcatagtcttcagcaagg

SEQ ID NO: 32 - DNA, 19 bp, Artificial Sequence
  Synthetic primer
gcaggatgagaatggattc

SEQ ID NO: 33 - DNA, 20 bp, Artificial Sequence
  Synthetic primer
gaattagagctttttgcctc

SEQ ID NO: 34 - DNA, 23 bp, Artificial Sequence
  Synthetic primer
acatgcttgctcatagtcttcag

SEQ ID NO: 35 - DNA, 22 bp, Artificial Sequence
  Synthetic primer
catgcttgctcatagtcttcag

SEQ ID NO: 36 - DNA, 21 bp, Artificial Sequence
  SEQ ID No 37
atgcttgctcatagtcttcag

SEQ ID NO: 37 - DNA, 20 bp, Artificial Sequence
  Synthetic primer
tgcttgctcatagtcttcag

SEQ ID NO: 38 - DNA, 19 bp, Artificial Sequence
  Synthetic primer
gcttgctcatagtcttcag

SEQ ID NO: 39 - DNA, 22 bp, Artificial Sequence
  Synthetic primer
actatgagcaagcatgtcttta

SEQ ID NO: 40 - DNA, 21 bp, Artificial Sequence
  Synthetic primer
ctatgagcaagcatgtcttta

SEQ ID NO: 41 - DNA, 20 bp, Artificial Sequence
  Synthetic primer
tatgagcaagcatgtcttta

SEQ ID NO: 42 - DNA, 19 bp, Artificial Sequence
  Synthetic primer
atgagcaagcatgtcttta

SEQ ID NO: 43 - DNA, 18 bp, Artificial Sequence
  Synthetic primer
tgagcaagcatgtcttta

SEQ ID NO: 44 - DNA, 17 bp, Artificial Sequence
  Synthetic primer
gagcaagcatgtcttta

SEQ ID NO: 45 - DNA, 16 bp, Artificial Sequence
  Synthetic primer
agcaagcatgtcttta

SEQ ID NO: 46 - DNA, 22 bp, Artificial Sequence
  Synthetic primer
ttgttggtgatggagcctgtgt

SEQ ID NO: 47 - DNA, 21 bp, Artificial Sequence
  Synthetic primer
tgttggtgatggagcctgtgt

SEQ ID NO: 48 - DNA, 20 bp, Artificial Sequence
  Synthetic primer
gttggtgatggagcctgtgt

SEQ ID NO: 49 - DNA, 19 bp, Artificial Sequence
  Synthetic primer
ttggtgatggagcctgtgt

SEQ ID NO: 50 - DNA, 18 bp, Artificial Sequence
  Synthetic primer
tggtgatggagcctgtgt

SEQ ID NO: 51 - DNA, 17 bp, Artificial Sequence
  Synthetic primer
ggtgatggagcctgtgt

SEQ ID NO: 52 - DNA, 16 bp, Artificial Sequence
  Synthetic primer
gtgatggagcctgtgt

SEQ ID NO: 53 - DNA, 18 bp, Artificial Sequence
  Synthetic primer
ttctcaaacactgtgggc

SEQ ID NO: 54 - DNA, 19 bp, Artificial Sequence
  Synthetic primer
gaactggtccttgctgaag

SEQ ID NO: 55 - DNA, 20 bp, Artificial Sequence
  Synthetic primer
tctgccacatagttctcaaa

SEQ ID NO: 56 - DNA, 21 bp, Artificial Sequence
  Synthetic primer
tctgtgttttgtgtttcagca

SEQ ID NO: 57 - DNA, 20 bp, Artificial Sequence
  Synthetic primer
ctgtgttttgtgtttcagca

SEQ ID NO: 58 - DNA, 19 bp, Artificial Sequence
  Synthetic primer
tgtgttttgtgtttcagca

SEQ ID NO: 59 - DNA, 18 bp, Artificial Sequence
  Synthetic primer
gtgttttgtgtttcagca

SEQ ID NO: 60 - DNA, 23 bp, Artificial Sequence
  Synthetic primer
catgtctttccacaggctccatc

SEQ ID NO: 61 - DNA, 22 bp, Artificial Sequence
  Synthetic primer
atgtctttccacaggctccatc

SEQ ID NO: 62 - DNA, 21 bp, Artificial Sequence
  Synthetic primer
atgtctttccacaggctccat

SEQ ID NO: 63 - DNA, 20 bp, Artificial Sequence
  Synthetic primer
atgtctttccacaggctcca

SEQ ID NO: 64 - DNA, 19 bp, Artificial Sequence
  Synthetic primer
atgtctttccacaggctcc

SEQ ID NO: 65 - DNA, 23 bp, Artificial Sequence
  Synthetic primer
atggagcctgtggaaagacatgc

SEQ ID NO: 66 - DNA, 22 bp, Artificial Sequence
  Synthetic primer
atggagcctgtggaaagacatg

SEQ ID NO: 67 - DNA, 21 bp, Artificial Sequence
  Synthetic primer
atggagcctgtggaaagacat

SEQ ID NO: 68 - DNA, 20 bp, Artificial Sequence
  Synthetic primer
atggagcctgtggaaagaca

SEQ ID NO: 69 - DNA, 19 bp, Artificial Sequence
  Synthetic primer
tggagcctgtggaaagaca

SEQ ID NO: 70 - DNA, 19 bp, Artificial Sequence
  Synthetic primer
gacacatgctttggtaacc

SEQ ID NO: 71 - DNA, 18 bp, Artificial Sequence
  Synthetic primer
cttttggtgccaggtgga

SEQ ID NO: 72 - DNA, 22 bp, Artificial Sequence
  Synthetic primer
tatgagcaagcatgtctttcct

SEQ ID NO: 73 - DNA, 21 bp, Artificial Sequence
  Synthetic primer
atgagcaagcatgtctttcct

SEQ ID NO: 74 - DNA, 20 bp, Artificial Sequence
  Synthetic primer
tgagcaagcatgtctttcct

SEQ ID NO: 75 - DNA, 19 bp, Artificial Sequence
  Synthetic primer
gagcaagcatgtctttcct

SEQ ID NO: 76 - DNA, 18 bp, Artificial Sequence
  Synthetic primer
agcaagcatgtctttcct

SEQ ID NO: 77 - DNA, 17 bp, Artificial Sequence
  Synthetic primer
gcaagcatgtctttcct

SEQ ID NO: 78 - DNA, 16 bp, Artificial Sequence
  Synthetic primer
caagcatgtctttcct

SEQ ID NO: 79 - DNA, 22 bp, Artificial Sequence
  Synthetic primer
gattgttggtgatggagcctga

SEQ ID NO: 80 - DNA, 21 bp, Artificial Sequence
  Synthetic primer
attgttggtgatggagcctga

SEQ ID NO: 81 - DNA, 20 bp, Artificial Sequence
  Synthetic primer
ttgttggtgatggagcctga

SEQ ID NO: 82 - DNA, 19 bp, Artificial Sequence
  Synthetic primer
tgttggtgatggagcctga

SEQ ID NO: 83 - DNA, 18 bp, Artificial Sequence
  Synthetic primer
gttggtgatggagcctga

SEQ ID NO: 84 - DNA, 17 bp, Artificial Sequence
  Synthetic primer
ttggtgatggagcctga

SEQ ID NO: 85 - DNA, 16 bp, Artificial Sequence
  Synthetic primer
tggtgatggagcctga

SEQ ID NO: 86 - DNA, 20 bp, Artificial Sequence
  Synthetic primer
gtgttttgtgtttcagcaat

SEQ ID NO: 87 - DNA, 21 bp, Artificial Sequence
  Synthetic primer
gtgttttgtgtttcagcaatg

SEQ ID NO: 88 - DNA, 20 bp, Artificial Sequence
  Synthetic primer
atagttctcaaacactgtgg

SEQ ID NO: 89 - DNA, 22 bp, Artificial Sequence
  Synthetic primer
aagcatgtctttcctcaggcta

SEQ ID NO: 90 - DNA, 21 bp, Artificial Sequence
  Synthetic primer
agcatgtctttcctcaggcta

SEQ ID NO: 91 - DNA, 20 bp, Artificial Sequence
  Synthetic primer
gcatgtctttcctcaggcta

SEQ ID NO: 92 - DNA, 19 bp, Artificial Sequence
  Synthetic primer
catgtctttcctcaggcta

SEQ ID NO: 93 - DNA, 18 bp, Artificial Sequence
  Synthetic primer
atgtctttcctcaggcta

SEQ ID NO: 94 - DNA, 17 bp, Artificial Sequence
  Synthetic primer
tgtctttcctcaggcta

SEQ ID NO: 95 - DNA, 16 bp, Artificial Sequence
  Synthetic primer
gtctttcctcaggcta

SEQ ID NO: 96 - DNA, 22 bp, Artificial Sequence
  Synthetic primer
aactggtgattgttggtgatgt

SEQ ID NO: 97 - DNA, 21 bp, Artificial Sequence
  Synthetic primer
actggtgattgttggtgatgt

SEQ ID NO: 98 - DNA, 20 bp, Artificial Sequence
  Synthetic primer
ctggtgattgttggtgatgt

SEQ ID NO: 99 - DNA, 19 bp, Artificial Sequence
  Synthetic primer
tggtgattgttggtgatgt

SEQ ID NO: 100 - DNA, 18 bp, Artificial Sequence
  Synthetic primer
ggtgattgttggtgatgt

SEQ ID NO: 101 - DNA, 17 bp, Artificial Sequence
  Synthetic primer
gtgattgttggtgatgt

SEQ ID NO: 102 - DNA, 16 bp, Artificial Sequence
  Synthetic primer
tgattgttggtgatgt

SEQ ID NO: 103 - DNA, 23 bp, Artificial Sequence
  Synthetic primer
ttccacaggctccatcaccaaca

SEQ ID NO: 104 - DNA, 22 bp, Artificial Sequence
  Synthetic primer
ttccacaggctccatcaccaac

SEQ ID NO: 105 - DNA, 21 bp, Artificial Sequence
  Synthetic primer
tccacaggctccatcaccaac

SEQ ID NO: 106 - DNA, 20 bp, Artificial Sequence
  Synthetic primer
tccacaggctccatcaccaa

SEQ ID NO: 107 - DNA, 19 bp, Artificial Sequence
  Synthetic primer
ccacaggctccatcaccaa

SEQ ID NO: 108 - DNA, 23 bp, Artificial Sequence
  Synthetic primer
tgttggtgatggagcctgtggaa

SEQ ID NO: 109 - DNA, 22 bp, Artificial Sequence
  Synthetic primer
tgttggtgatggagcctgtgga

SEQ ID NO: 110 - DNA, 21 bp, Artificial Sequence
  Synthetic primer
gttggtgatggagcctgtgga

SEQ ID NO: 111 - DNA, 20 bp, Artificial Sequence
  Synthetic primer
ttggtgatggagcctgtgga

SEQ ID NO: 112 - DNA, 19 bp, Artificial Sequence
  Synthetic primer
ttggtgatggagcctgtgg

SEQ ID NO: 113 - DNA, 16 bp, Artificial Sequence
  Synthetic primer
gcacatacacctctgg

SEQ ID NO: 114 - DNA, 17 bp, Artificial Sequence
  Synthetic primer
tgattgttggtgatgga

SEQ ID NO: 115 - DNA, 18 bp, Artificial Sequence
  Synthetic primer
ccttgctgaagactatga

SEQ ID NO: 116 - DNA, 19 bp, Artificial Sequence
  Synthetic primer
gtgcattgcaggtaatatc

SEQ ID NO: 117 - DNA, 20 bp, Artificial Sequence
  Synthetic primer
ctgaagactatgagcaagca

SEQ ID NO: 118 - DNA, 19 bp, Artificial Sequence
  Synthetic primer
ctgaggactatgagcaagc

SEQ ID NO: 119 - DNA, 19 bp, Artificial Sequence
  Synthetic primer
catgtctttccacaggctc

SEQ ID NO: 120 - DNA, 19 bp, Artificial Sequence
  Synthetic primer
catgtctttacacaggctc

SEQ ID NO: 121 - DNA, 20 bp, Artificial Sequence
  Synthetic primer
tgtctttccacaggctccat

SEQ ID NO: 122 - DNA, 20 bp, Artificial Sequence
  Synthetic primer
tgtctttcctcaggctccat

SEQ ID NO: 123 - DNA, 19 bp, Artificial Sequence
  Synthetic primer
cacaggctccatcaccaac

SEQ ID NO: 124 - DNA, 19 bp, Artificial Sequence
  Synthetic primer
cacaggctacatcaccaac

SEQ ID NO: 125 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 126 - DNA, 1926 bp, Homo sapiens
gtggatgagctgtgagtgcgcgcgcgtgcgcggggccgcgacctgtgccggctcgagcccgctgggcactcggaggcgcgcacgtcgttccccgccctcccgccgccgcccgccctcgctctctcgcgctaccctcccgccgcccgcggtcctccgtcggttctctcgttagtccacggtctggtcttcagctacccgccttcgtctccgagtttgcgactcgcggaccggcgtccccggcgcgaagaggctggactcggattcgttgcctgagcaatggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctgggtgccttgtcttgtgaaaccttgctgcaagcacagcccttatgcggttaattttgaagtgctgtttattaatcttagtgtatgattactggcctttttcatttatctataatttacctaagattacaaatcagaagtcatcttgctaccagtatttagaagccaactatgattattaacgatgtccaacccgtctggcccaccagggtccttttgacactgctctaacagccctcctctgcactcccacctgacacaccaggcgctaattcaaggaatttcttaacttcttgcttctttctagaaagagaaacagttggtaacttttgtgaattaggctgtaactactttataactaacatgtcctgcctattatctgtcagctgcaaggtactctggtgagtcaccacttcagggctttactccgtaacagattttgttggcatagctctggggtgggcagttttttgaaaatgggctcaaccagaaaagcccaagttcatgcagctgtggcagagttacagttctgtggtttcatgttagttaccttatagttactgtgtaattagtgccacttaatgtatgttaccaaaaataaatatatctaccccagactagatgtagtattttttgtataattggatttcctaatactgtcatcctcaaagaaagtgtattggttttttaaaaaagaaagtgtatttggaaataaagtcagatggaaaattcattttttaaattcccgttttgtcactttttctgataaaagatggccatattaccccttttcggccccatgtatctcagtaccccatggagctgggctaagtaaataggaattggtttcacgcctgaggcaattagacactttggaagatggcataacctgtctcacctggacttaagcatctggctctaattcacagtgctcttttctcctcactgtatccaggttccctcccagaggagccaccagttctcatgggtggcactcagtctctcttctctccagctgactaaactttttttctgtaccagttaatttttccaactactaatagaataaaggcagttttctaaaaaaa

SEQ ID NO: 127 - DNA, 59 bp, Homo sapiens
ccggaagaaactggtgattgttggtgatgtagcctgtggaaagacatgcttgctcatag

SEQ ID NO: 128 - DNA, 59 bp, Homo sapiens
ctatgagcaagcatgtctttccacaggctacatcaccaacaatcaccagtttcttccgg

SEQ ID NO: 129 - DNA, 59 bp, Homo sapiens
ccggaagaaactggtgattgttggtgatgaagcctgtggaaagacatgcttgctcatag

SEQ ID NO: 130 - DNA, 59 bp, Homo sapiens
ctatgagcaagcatgtctttccacaggcttcatcaccaacaatcaccagtttcttccgg

SEQ ID NO: 131 - DNA, 59 bp, Homo sapiens
actggtgattgttggtgatggagcctgtgtaaagacatgcttgctcatagtcttcagca

SEQ ID NO: 132 - DNA, 59 bp, Homo sapiens
tgctgaagactatgagcaagcatgtctttacacaggctccatcaccaacaatcaccagt

SEQ ID NO: 133 - DNA, 59 bp, Homo sapiens
cctgtggaaagacatgcttgctcatagtcctcagcaaggaccagttcccagaggtgtat

SEQ ID NO: 134 - DNA, 59 bp, Homo sapiens
atacacctctgggaactggtccttgctgaggactatgagcaagcatgtctttccacagg

SEQ ID NO: 135 - DNA, 108 bp, Artificial Sequence
  Synthetic primer
ttccatccacctcgatatctgccacatagttctcaaacactgtgggcacatacacctctgggaactggtccttgctgaggactatgagcaagcatgtctttcgatatc

SEQ ID NO: 136 - DNA, 22 bp, Artificial Sequence
  Synthetic primer
ggactatgagcaagcatgtctt

SEQ ID NO: 137 - DNA, 74 bp, Artificial Sequence
  Synthetic primer
gaactggtccttgctgaggactatgagcaagcatgtctttccacaggctccatcaccaacaatcaccagtttct

SEQ ID NO: 138 - DNA, 73 bp, Artificial Sequence
  Synthetic primer
aactggtccttgctgaggactatgagcaagcatgtctttccacaggctccatcaccaacaatcaccagtttct

SEQ ID NO: 139 - DNA, 72 bp, Artificial Sequence
  Synthetic primer
actggtccttgctgaggactatgagcaagcatgtctttccacaggctccatcaccaacaatcaccagtttct

SEQ ID NO: 140 - DNA, 76 bp, Artificial Sequence
  Synthetic primer
ccacatagttctcaaacactgtgggcacatacacctctgggaactggtccttgctgaggactatgagcaagcatgt

SEQ ID NO: 141 - DNA, 75 bp, Artificial Sequence
  Synthetic primer
cacatagttctcaaacactgtgggcacatacacctctgggaactggtccttgctgaggactatgagcaagcatgt

SEQ ID NO: 142 - DNA, 74 bp, Artificial Sequence
  Synthetic primer
cacatagttctcaaacactgtgggcacatacacctctgggaactggtccttgctgaggactatgagcaagcatg

SEQ ID NO: 143 - DNA, 73 bp, Artificial Sequence
  Synthetic primer
acatagttctcaaacactgtgggcacatacacctctgggaactggtccttgctgaggactatgagcaagcatg

SEQ ID NO: 144 - DNA, 73 bp, Artificial Sequence
  Synthetic primer
cacatagttctcaaacactgtgggcacatacacctctgggaactggtccttgctgaggactatgagcaagcat

SEQ ID NO: 145 - DNA, 72 bp, Artificial Sequence
  Synthetic primer
acatagttctcaaacactgtgggcacatacacctctgggaactggtccttgctgaggactatgagcaagcat

SEQ ID NO: 146 - DNA, 71 bp, Artificial Sequence
  Synthetic primer
acatagttctcaaacactgtgggcacatacacctctgggaactggtccttgctgaggactatgagcaagca

SEQ ID NO: 147 - DNA, 70 bp, Artificial Sequence
  Synthetic primer
catagttctcaaacactgtgggcacatacacctctgggaactggtccttgctgaggactatgagcaagca

SEQ ID NO: 148 - DNA, 46 bp, Homo sapiens
cctctgggaactggtccttgctgaagactatgagcaagcatgtctt

SEQ ID NO: 149 - DNA, 46 bp, Homo sapiens
aagacatgcttgctcatagtcttcagcaaggaccagttcccagagg

SEQ ID NO: 150 - DNA, 90 bp, Artificial Sequence
  Synthetic primer
tatgagcaagcatgtctttacacaggctccatcaccaacaatcaccagtttcttccggatggcagccattgctgaaacacaaaacacaga

SEQ ID NO: 151 - DNA, 89 bp, Artificial Sequence
  Synthetic primer
tatgagcaagcatgtctttacacaggctccatcaccaacaatcaccagtttcttccggatggcagccattgctgaaacacaaaacacag

SEQ ID NO: 152 - DNA, 89 bp, Artificial Sequence
  Synthetic primer
atgagcaagcatgtctttacacaggctccatcaccaacaatcaccagtttcttccggatggcagccattgctgaaacacaaaacacaga

SEQ ID NO: 153 - DNA, 88 bp, Artificial Sequence
  Synthetic primer
atgagcaagcatgtctttacacaggctccatcaccaacaatcaccagtttcttccggatggcagccattgctgaaacacaaaacacag

SEQ ID NO: 154 - DNA, 87 bp, Artificial Sequence
  Synthetic primer
tgagcaagcatgtctttacacaggctccatcaccaacaatcaccagtttcttccggatggcagccattgctgaaacacaaaacacag

SEQ ID NO: 155 - DNA, 86 bp, Artificial Sequence
  Synthetic primer
tgagcaagcatgtctttacacaggctccatcaccaacaatcaccagtttcttccggatggcagccattgctgaaacacaaaacaca

SEQ ID NO: 156 - DNA, 85 bp, Artificial Sequence
  Synthetic primer
gagcaagcatgtctttacacaggctccatcaccaacaatcaccagtttcttccggatggcagccattgctgaaacacaaaacaca

SEQ ID NO: 157 - DNA, 84 bp, Artificial Sequence
  Synthetic primer
gagcaagcatgtctttacacaggctccatcaccaacaatcaccagtttcttccggatggcagccattgctgaaacacaaaacac

SEQ ID NO: 158 - DNA, 90 bp, Artificial Sequence
  Synthetic primer
ttctcaaacactgtgggcacatacacctctgggaactggtccttgctgaagactatgagcaagcatgtctttacacaggctccatcacca

SEQ ID NO: 159 - DNA, 58 bp, Artificial Sequence
  Synthetic primer
gaactggtccttgctgaagactatgagcaagcatgtctttacacaggctccatcacca

SEQ ID NO: 160 - DNA, 89 bp, Artificial Sequence
  Synthetic primer
ttctcaaacactgtgggcacatacacctctgggaactggtccttgctgaagactatgagcaagcatgtctttacacaggctccatcacc

SEQ ID NO: 161 - DNA, 57 bp, Artificial Sequence
  Synthetic primer
gaactggtccttgctgaagactatgagcaagcatgtctttacacaggctccatcacc

SEQ ID NO: 162 - DNA, 100 bp, Artificial Sequence
  Synthetic primer
tctgccacatagttctcaaacactgtgggcacatacacctctgggaactggtccttgctgaagactatgagcaagcatgtctttacacaggctccatcac

SEQ ID NO: 163 - DNA, 42 bp, Homo sapiens
tatgagcaagcatgtctttccacaggctccatcaccaacaat

SEQ ID NO: 164 - DNA, 42 bp, Homo sapiens
attgttggtgatggagcctgtggaaagacatgcttgctcata

SEQ ID NO: 165 - DNA, 85 bp, Artificial Sequence
  SEQ ID No 165
gagcaagcatgtctttcctcaggctccatcaccaacaatcaccagtttcttccggatggcagccattgctgaaacacaaaacaca

SEQ ID NO: 166 - DNA, 84 bp, Artificial Sequence
  Synthetic primer
gagcaagcatgtctttcctcaggctccatcaccaacaatcaccagtttcttccggatggcagccattgctgaaacacaaaacac

SEQ ID NO: 167 - DNA, 92 bp, Artificial Sequence
  Synthetic primer
ttctcaaacactgtgggcacatacacctctgggaactggtccttgctgaagactatgagcaagcatgtctttcctcaggctccatcaccaac

SEQ ID NO: 168 - DNA, 60 bp, Artificial Sequence
  Synthetic primer
gaactggtccttgctgaagactatgagcaagcatgtctttcctcaggctccatcaccaac

SEQ ID NO: 169 - DNA, 91 bp, Artificial Sequence
  Synthetic primer
ttctcaaacactgtgggcacatacacctctgggaactggtccttgctgaagactatgagcaagcatgtctttcctcaggctccatcaccaa

SEQ ID NO: 170 - DNA, 59 bp, Artificial Sequence
  Synthetic primer
gaactggtccttgctgaagactatgagcaagcatgtctttcctcaggctccatcaccaa

SEQ ID NO: 171 - DNA, 90 bp, Artificial Sequence
  Synthetic primer
ttctcaaacactgtgggcacatacacctctgggaactggtccttgctgaagactatgagcaagcatgtctttcctcaggctccatcacca

SEQ ID NO: 172 - DNA, 58 bp, Artificial Sequence
  Synthetic primer
gaactggtccttgctgaagactatgagcaagcatgtctttcctcaggctccatcacca

SEQ ID NO: 173 - DNA, 96 bp, Artificial Sequence
  Synthetic primer
atagttctcaaacactgtgggcacatacacctctgggaactggtccttgctgaagactatgagcaagcatgtctttcctcaggctccatcaccaac

SEQ ID NO: 174 - DNA, 81 bp, Artificial Sequence
  Synthetic primer
aagcatgtctttcctcaggctacatcaccaacaatcaccagtttcttccggatggcagccattgctgaaacacaaaacaca

SEQ ID NO: 175 - DNA, 80 bp, Artificial Sequence
  Synthetic primer
aagcatgtctttcctcaggctacatcaccaacaatcaccagtttcttccggatggcagccattgctgaaacacaaaacac

SEQ ID NO: 176 - DNA, 100 bp, Artificial Sequence
  Synthetic primer
ttctcaaacactgtgggcacatacacctctgggaactggtccttgctgaagactatgagcaagcatgtctttccacaggctccatcaccaacaatcacca

SEQ ID NO: 177 - DNA, 68 bp, Artificial Sequence
  Synthetic primer
gaactggtccttgctgaagactatgagcaagcatgtctttccacaggctccatcaccaacaatcacca

SEQ ID NO: 178 - DNA, 104 bp, Artificial Sequence
  Synthetic primer
atagttctcaaacactgtgggcacatacacctctgggaactggtccttgctgaagactatgagcaagcatgtctttccacaggctccatcaccaacaatcacca

SEQ ID NO: 179 - DNA, 81 bp, Artificial Sequence
  Synthetic primer
gcacatacacctctgggaactggtccttgctgaagactatgagcaagcatgtctttccacaggctccatcaccaacaatca

SEQ ID NO: 180 - DNA, 120 bp, Artificial Sequence
  Synthetic primer
ccttgctgaagactatgagcaagcatgtctttccacaggctccatcaccaacaatcaccagtttcttccggatggcagccattgctgaaacacaaaacacagatattacctgcaatgcac

SEQ ID NO: 181 - DNA, 244 bp, Homo sapiens
ctgcctggctgtgtcccacaaagtcagctctacactttcccatccacctcgatatgggccacatagttctgaaaccctgagggtgctcacccctctgggaactggtccttgctgaagacaatgagcaagcatgtcttgccacagactctgtcaccgacaattaccagcttcttctggacgggaaccattgctggaggacactggcctgggtggctgaaattgtacgaagtaccttgaacttctc

SEQ ID NO: 182 - DNA, 274 bp, Homo sapiens
gtgtccgagtaaaagaggggcctcaggcgatcctaatcttcctgcctggctgtgtcccacaaagtcagctctacactttcccatccacctcgatatgggccacatagttctgaaaccctgagggtgctcacccctctgggaactggtccttgctgaagacaatgagcaagcatgtcttgccacagactctgtcaccgacaattaccagcttcttctggacgggaaccattgctggaggacactggcctgggtggctgaaattgtacgaagtacc

SEQ ID NO: 183 - DNA, 220 bp, Homo sapiens
tatcagattccattggccgagaagctcaaggctccagcaccggccttgaaggatggacagactttgaatggggggcgtggcctaaacagggacggggttgttgctgaaacacaaaacacactgaaagagaacatggggttaaaaaaggctctcagtgatgcaaagcggcttaaaactggggaagaaaggggcaggtctactcccgggctgtacattctgc

SEQ ID NO: 184 - DNA, 239 bp, Homo sapiens
tcctgcctggctgtgtcccacaaagtcagctctacactttcccatccacctcgatatgggccacatagttctgaaaccctgagggtgctcacccctctgggaactggtccttgctgaagacaatgagcaagcatgtcttgccacagactctgtcaccgacaattaccagcttcttctggacgggaaccattgctggaggacactggcctgggtggctgaaattgtacgaagtaccttga

SEQ ID NO: 185 - DNA, 220 bp, Homo sapiens; identical to SEQ ID NO: 183

SEQ ID NO: 186 - DNA, 258 bp, Homo sapiens
aaatggcatcagttgttatgaaaagtatactcacctgctttccatccacctcgatatctgccacatagttctcaaacactgtgggcacatacacctctgggaactggtccttgctgaagactatgagcaagcatgtctttccacaggctccatcaccaacaatcaccagtttcttccggatggcagccattgctgaaacacaaaacacagatattacctgcaatgcacaagaagtcataggtagcttacaggatgaca

SEQ ID NO: 187 - DNA, 225 bp, Homo sapiens
tttgaaaaatccagtgtctagatgggcatcggacagatgctggatcctctaaaactgagatggagctaaatggggggttggagcagttaatgaattggagagccatttgctgaaacacaaaacccaaggagagaggcaccatttggttgtgattatttaaaacccagacttcaatgtctcaaaaataatttagcatggacattggaaactgagtgtctgtgggta

SEQ ID NO: 188 - DNA, 253 bp, Homo sapiens
ctaatcttcctgcctggctgtgtcccacaaagtcagctctacactttcccatccacctcgatatgggccacatagttctgaaaccctgagggtgctcacccctctgggaactggtccttgctgaagacaatgagcaagcatgtcttgccacagactctgtcaccgacaattaccagcttcttctggacgggaaccattgctggaggacactggcctgggtggctgaaattgtacgaagtaccttgaacttctc

SEQ ID NO: 189 - DNA, 238 bp, Homo sapiens
ggctgtgtcccacaaagtcagctctacactttcccatccacctcgatatgggccacatagttctgaaaccctgagggtgctcacccctctgggaactggtccttgctgaagacaatgagcaagcatgtcttgccacagactctgtcaccgacaattaccagcttcttctggacgggaaccattgctggaggacactggcctgggtggctgaaattgtacgaagtaccttgaacttctc